(19) United States
(12) Patent Application Publication
(10) Pub. No.: US 2022/0407026 A1
(43) Pub. Date: Dec. 22, 2022

(54) ORGANIC ELECTROLUMINESCENT DEVICE

(71) Applicant: BEIJING SUMMER SPROUT TECHNOLOGY CO., LTD., Beijing (CN)

(72) Inventors: Huiqing PANG, Beijing (CN); Jing WANG, Beijing (CN); Zhihao CUI, Beijing (CN); Hualong DING, Beijing (CN); Renjie ZHENG, Beijing (CN); Chi Yuen Raymond KWONG, Beijing (CN)

(21) Appl. No.: 17/825,854

(22) Filed: May 26, 2022

(30) Foreign Application Priority Data
May 28, 2021 (CN) 202110592096.5
Apr. 7, 2022 (CN) 202210355382.4

(51) Int. Cl. H01L 51/50; C07D 209/82; C07D 409/12; C07D 401/12; C07D 405/12; C07D 403/12; C07D 401/04; C07D 405/04; C07D 409/04; C07D 403/14; C07C 211/54; C07C 15/28; C07D 487/16; C07C 255/51; C07D 498/04; H01L 51/00

(52) U.S. Cl. CPC H01L 51/5004; C07D 209/82; C07D 409/12; C07D 401/12; C07D 405/12; C07D 403/12; C07D 401/04; C07D 405/04; C07D 409/04; C07D 403/14; C07C 211/54; C07C 15/28; C07D 487/16; C07C 255/51; C07D 498/04; H01L 51/0052; H01L 51/0059; H01L 51/0064; H01L 51/0069; H01L 51/0071; H01L 51/0072; H01L 51/0073; H01L 51/0074; H01L 51/0091; H01L 51/0079; H01L 51/0083; H01L 51/5012; H01L 2251/558; H01L 2251/552; H01L 51/5092

(57) ABSTRACT

The device provided herein is an organic electroluminescent device and includes a substrate; a first electrode on the substrate and with high reflectivity; a translucent or transparent second electrode over the first electrode; and a first, a second and a third organic layer included between the first and the second electrode; where the second organic layer has a thickness >80 nm and is made of a second organic material; the third organic layer is a light-emitting layer including at least one light-emitting material and at least one host material; the first organic layer has a conductivity >1×10⁻⁴ S/m and <1×10⁻² S/m; an energy level difference between HOMO energy level of the second organic material and HOMO energy level of the at least one host material is <0.27 eV; and the first electrode and the second organic layer are in direct contact with the first organic layer.

### CROSS-REFERENCE TO RELATED APPLICATION(S)

This application claims priority to Chinese Patent Application No. CN 202110592096.5 filed on May 28, 2021 and Chinese Patent Application No. CN 202210355382.4 filed on Apr. 7, 2022, the disclosure of which are incorporated herein by reference in their entireties.

### TECHNICAL FIELD

The present disclosure relates to an organic electroluminescent device and a display assembly including the organic electroluminescent device.

### BACKGROUND

Organic electroluminescent devices (such as organic light-emitting diodes (OLEDs)) have been developed for nearly three decades from a double organic layer structure originally reported by Tang and Van Slyke of Eastman Kodak (Applied Physics Letters, 1987, 51 (12): 913-915) to a structure having 6 to 7 function layers, which is widely commercialized at present. The introduction of various function layers greatly improves the transport performance of carriers, and materials of different function layers may be selected to control the balance of carriers, thus greatly improving device performance. However, the introduction of more function layers and materials thereof requires more process steps and more vacuum chambers, which inevitably increases a production cost. Additionally, more interfaces result from more function layers, and an interface is generally a weak link in a carrier transporting process due to the existence of defects, which often affects the device performance (Jiang Y, Zhou D Y, Dong S C, et al. 19-2: Sid Symposium Digest of Technical Papers, 2019) (H. Yamamoto et al., 52.3, 758•SID 2014 DIGEST). Therefore, if the device structure can be simplified and the number of film layers and/or materials can be reduced on the premise that the device performance is basically maintained, the production cost can be effectively reduced.

The currently commercialized device structure includes a cathode, an anode and a series of organic function layers arranged between the cathode and anode, where the organic function layers include a hole injection layer (HIL), a hole transporting layer (HTL), an electron blocking layer (EBL), an emissive layer (EML), a hole blocking layer (HBL), an electron transporting layer (ETL) and an electron injection layer (EIL), etc. The HIL is generally made of a hole transporting material (HTM) doped with a low proportion of conductive p-type doping material (PD), where a doping ratio is generally from 1% to 3%. The HTL is generally made of the HTM used in the HIL. The emissive layer is generally made of at least one host material and at least one light-emitting material. Some emissive layers may adopt a dual-host architecture, and an emissive layer emitting yellow or white light may adopt a dual-light-emitting material architecture. Generally speaking, the host material in the emissive layer has a deeper HOMO energy level than the HTM so that holes face a relatively high potential barrier if they travel directly from the HTL to the EML. To solve this problem, the EBL (also known as a prime layer or a second hole transporting layer) is introduced, which has a HOMO energy level between those of the HTM and the host material. To simplify the device structure, a feasible idea is to combine the HTL and the EBL into one layer and use an HTM with a deep energy level to connect the HIL and the EML. This results in the problem that the HTM with a deep energy level needs to be doped with a PD material with a deeper LUMO energy level such that the HTM has a good hole injection ability. However, the currently commercialized PD material has a LUMO energy level of −5.05 eV and cannot be effectively used as the p-type doping material for the HTM with a deep energy level.

CN201911209540X is a previous application of the applicant and discloses that a PD material with a relatively deep LUMO energy level is doped into a hole transporting material (HTM) with a relatively deep HOMO energy level, which are co-deposited as a hole injection layer (HIL) used in a bottom-emitting device emitting blue light. Due to better matched energy levels and reduced film layers and materials, the device has a reduced voltage and a prolonged lifetime and the process is simplified. However, this application adopts a bottom-emitting device, and the cathode, anode and electron injection layer thereof are all different from those of a top-emitting device, which brings about a difference in carrier distribution in the system of the device. CN2021101318064 is a previous application of the applicant and discloses an embodiment in which simple structures are vertically stacked to form a device with stacked layers, and the device obtains good performance. In this application, multiple light-emitting units are arranged in a physical form of being vertically stacked so that the circuit has a series characteristic. Such OLEDs are referred to as stacked OLEDs (in terms of the physical form) or series OLEDs (in terms of a circuit connection). However, the structure of the top-emitting device is optimized in neither of the above applications. When the structure of the top-emitting device is optimized, the HTM has a larger thickness and the performance, especially electrical performance, of other relevant function layers must be comprehensively investigated to meet the requirement of the device for a low voltage, which is not mentioned in the above applications.

At present, the most commonly used device structure in display applications is the top-emitting device. Generally, thicker HTL and EBL are used so as to adjust a microcavity effect and achieve a target color. For example, the total thickness of the HTL and the EBL in the top-emitting device emitting red or green light is generally around 180-190 nm. If the HTM with a deep HOMO energy level is used in such a thick film layer, the voltage will rise sharply with certainty so that the device performance is seriously affected. Additionally, the cathode, anode and electron injection layer of the top-emitting device all use different materials from those of the bottom-emitting device. For example, a conventional bottom-emitting device uses Liq with a thickness of 1-2 nm as the EIL and Al (opaque) with a thickness of above 100 nm as the cathode; and the top-emitting device generally uses Yb with a thickness of 1-2 nm as the EIL and a Mg—Ag alloy (translucent, a ratio of Mg:Ag generally being 1:9) with a thickness of 10-15 nm as the cathode. In this manner, the bottom-emitting device and the top-emitting device have different electron injection situations so that the whole device systems have different carrier balance situations. Differences lie in not only the cathodes but also the anodes. Though ITO anodes are used in both the bottom-emitting device and the top-emitting device, the ITO layer used for hole injection in the top-emitting device is generally very thin and typically has a thickness of 5-20 nm while the ITO layer in the bottom-emitting device typically has a thickness of 80-120 nm. ITO layers with different thicknesses have different surface roughness, which also affects hole injection. Moreover, the ITO anodes in the top-emitting device and the bottom-emitting device are generally prepared by different processes so that a deviation is introduced into the work function of ITO, which further affects hole injection. Therefore, the practice of simple structures in the top-emitting device requires re-optimization and selection of materials.

Additionally, the HIL in a conventional top-emitting device is generally in the form of an HTM doped with a PD material and typically has a thickness of 10 nm and a conductivity of 1×10⁻³ S/m to ensure a good hole injection ability, and accordingly the selected HTM has a relatively shallow HOMO energy level which is generally around −5.1 eV. The commonly used host material in the emissive layer has a HOMO energy level of about −5.4 eV and lower so that an energy level difference of greater than about 0.3 eV is formed between the HTM and the host material, which affects hole transport. Though sufficient holes are injected from the anode to the HIL, the transport of holes from the HIL to the EML is limited by a high potential barrier so that the EBL needs to be added for potential barrier transition, which increases the production cost and complexity and generally affects the device performance to a certain degree. On the other hand, a large number of holes are injected from the HIL to the HTL and further transported to the EBL or EML. However, due to a relatively high potential barrier at the interface, holes accumulate at the interface, resulting in excessive holes, which also affects the device performance. In the present disclosure, researches show that an HTM with a relatively deep HOMO energy level is selected in the device to match the energy level of the host material and disposed between the HIL and the EML to reduce the potential barrier and reduce film layers, which can reduce the production cost and improve the device performance. Meanwhile, a PD material with a deep energy level is doped into the HTM to ensure good hole injection. In this case, the conductivity may be reduced to 1×10⁻⁴ S/m. However, since the HTM doped with PD can better match the hole transporting layer and avoid the excess and accumulation of holes, the voltage can be reduced and the lifetime can be prolonged in the case where the efficiency is basically unchanged. In fact, lower conductivity is conducive to reducing the risk of crosstalk between pixels in the device.

### SUMMARY

The present disclosure aims to provide an organic electroluminescent device to solve at least part of the above problems.

According to an embodiment of the present disclosure, an organic electroluminescent device is disclosed. The organic electroluminescent device comprises:

a substrate;

a first electrode disposed on the substrate;

a second electrode disposed over the first electrode; and

an organic layer disposed between the first electrode and the second electrode;

wherein the first electrode is a material with high reflectivity or a combination of materials with high reflectivity, and the second electrode is a translucent or transparent material or a combination of translucent or transparent materials;

the organic layer comprises a first organic layer, a second organic layer and a third organic layer;

the first organic layer comprises a first organic material and a second organic material;

the second organic layer is made of the second organic material and has a thickness of greater than 80 nm;

the third organic layer is a light-emitting layer comprising at least one light-emitting material and at least one host material;

the first organic layer has a conductivity of greater than 1×10⁻⁴ S/m and less than 1×10⁻² S/m;

an energy level difference between a HOMO energy level of the second organic material and a HOMO energy level of the at least one host material is less than 0.27 eV;

one side of the first organic layer is in direct contact with the first electrode, and the other side of the first organic layer is in direct contact with the second organic layer.

According to an embodiment of the present disclosure, a first organic electroluminescent device is disclosed. The first organic electroluminescent device comprises:

a substrate;

a first electrode disposed on the substrate;

a second electrode disposed over the first electrode; and

an organic layer disposed between the first electrode and the second electrode;

wherein the first electrode is a material with high reflectivity or a combination of materials with high reflectivity, and the second electrode is a translucent or transparent material or a combination of translucent or transparent materials;

the organic layer comprises a first organic layer, a second organic layer and a third organic layer;

the first organic layer comprises a first organic material and a second organic material;

the second organic layer is made of the second organic material and has a first thickness;

the third organic layer is a light-emitting layer comprising at least one light-emitting material and at least one host material;

the first organic layer has a conductivity of greater than 1×10⁻⁴ S/m and less than 1×10⁻² S/m;

an energy level difference between a HOMO energy level of the second organic material and a HOMO energy level of the at least one host material is less than 0.27 eV;

a voltage of the first organic electroluminescent device is not higher than 110% of a voltage of a second organic electroluminescent device at the same current density, wherein the second organic electroluminescent device has the same device structure as the first organic electroluminescent device except the following differences:

(1) the first organic layer comprises the first organic material and a third organic material, wherein the third organic material is different from the second organic material;

(2) the second organic layer is made of the third organic material;

(3) a fourth organic layer is comprised between the second organic layer and the third organic layer, wherein the fourth organic layer is made of the second organic material;

wherein a total thickness of the second organic layer and the fourth organic layer in the second organic electroluminescent device is 90% to 110% of the first thickness in the first organic electroluminescent device.

According to another embodiment of the present disclosure, a display assembly is further disclosed. The display assembly comprises the preceding organic electroluminescent device.

According to another embodiment of the present disclosure, a display assembly is further disclosed. The display assembly comprises the preceding first organic electroluminescent device.

The present disclosure discloses an organic electroluminescent device which is an organic electroluminescent device with top emission. The organic electroluminescent device achieves good device performance, such as a reduced device voltage and a prolonged lifetime, by optimizing electrical performance of function layers, such as conductivity of a hole injection layer and an energy level difference between a hole transporting material and a host material in a light-emitting layer.

### BRIEF DESCRIPTION OF DRAWINGS

FIG. 1 is a structural diagram of a typical top-emitting OLED device.

FIG. 2 is a structural diagram of a simplified top-emitting device.

### DETAILED DESCRIPTION

An OLED device generally includes an anode layer, a hole injection layer (HIL), a hole transporting layer (HTL), an electron blocking layer (EBL), an emissive layer (EML), a hole blocking layer (HBL), an electron transporting layer (ETL), an electron injection layer (EIL), a cathode layer and a capping layer. There are more examples for each of these layers. For example, a flexible and transparent substrate-anode combination is disclosed in U.S. Pat. No. 5,844,363, which is incorporated by reference in its entirety. An example of p-doped hole transporting layers is m-MTDATA doped with F₄-TCNQ at a molar ratio of 50:1, as disclosed in U.S. Patent Application Publication No. 2003/0230980 which is incorporated by reference in its entirety. Examples of host materials are described in U.S. Pat. No. 6,360,562 issued to Thompson et al., which is incorporated by reference in its entirety. An example of n-doped electron transporting layers is BPhen doped with Li at a molar ratio of 1:1, as disclosed in U.S. Patent Application Publication No. 2003/0230980 which is incorporated by reference in its entirety. U.S. Pat. Nos. 5,703,436 and 5,707,745, both which are incorporated by reference in their entireties, disclose examples of cathodes, including composite cathodes having a thin metal layer such as Mg:Ag and an overlying transparent, conductive, sputter-deposited ITO layer. The principle and use of blocking layers are described in detail in U.S. Pat. No. 6,097,147 and U.S. Patent Application Publication No. 2003/0230980, both which are incorporated by reference in their entireties. U.S. Patent Application Publication No. 2004/0174116 which is incorporated by reference in its entirety provides examples of injection layers. The description about protective layers can be found in U.S. Patent Application Publication No. 2004/0174116 which is incorporated by reference in its entirety.

The above-mentioned layered structure is provided via non-limiting embodiments. The function of the OLED can be implemented by combining the various layers described above, or some layers can be omitted. The OLED can also include other layers that are not explicitly described herein. In each layer, a single material or a mixture of multiple materials can be used to achieve the best performance. Any functional layer can include several sub-layers. For example, the light-emitting layer can have two different layers of light-emitting materials to achieve a desired light-emitting spectrum.

In an embodiment, the OLED can be described as an OLED having an “organic layer” disposed between the cathode and the anode. This organic layer can include one or more layers.

The device fabricated in accordance with embodiments of the present disclosure can be incorporated into a wide variety of consumer products that have one or more of the electronic component modules (or units) of this device. Some examples of such consumer products include flat panel displays, monitors, medical monitors, televisions, billboards, lights for indoor or outdoor lighting and/or signaling, head-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein can also be used in other organic electronic devices listed above.

As used herein, “top” means being located furthest away from the substrate while “bottom” means being located closest to the substrate. In a case where a first layer is described as “being disposed over” a second layer, the first layer is disposed further away from the substrate. There may be other layers between the first and second layers, unless it is specified that the first layer is “in contact with” the second layer. For example, a cathode can still be described as “being disposed over” an anode, even though there are various organic layers between the cathode and the anode.

“Solution processible”, as used herein, means that capable of being dissolved, dispersed or transported in a liquid medium in the form of a solution or suspension and/or deposited from a liquid medium.

The work function of the metal herein refers to the minimum amount of energy required to move an electron from the interior to the surface of an object. All “work functions of the metal” herein are expressed as negative values, that is, the smaller the numerical value (i.e., the larger the absolute value), the larger amount of energy required to pull the electron to the vacuum level. For example, “the work function of the metal is less than −5 eV” means that the amount of energy required to pull the electron to the vacuum level is greater than 5 eV.

Herein, the numerical values of a highest occupied molecular orbital (HOMO) energy level and a lowest occupied molecular orbital (LUMO) energy level are measured through electrochemical cyclic voltammetry, which is the most commonly used method of measuring energy levels of organic materials. The test is conducted using an electrochemical workstation modelled CorrTest CS120 produced by Wuhan Corrtest Instruments Corp., Ltd and using a three-electrode working system where: a platinum disk electrode serves as a working electrode, a Ag/AgNO₃ electrode serves as a reference electrode, and a platinum wire electrode serves as an auxiliary electrode. Anhydrous DCM is used as a solvent, 0.1 mol/L tetrabutylammonium hexafluorophosphate is used as a supporting electrolyte, a compound to be tested is prepared into a solution of 10⁻³ mol/L, and nitrogen is introduced into the solution for 10 min for oxygen removal before the test. The parameters of the instrument are set as follows: a scan rate of 100 mV/s, a potential interval of 0.5 mV and a test window of −1 V to 1 V. Herein, all “HOMO energy levels” and “LUMO energy levels” are expressed as negative values, and the smaller the numerical value (i.e., the larger the absolute value), the deeper the energy level. In the present application, the expression that the energy level is smaller than a certain number means that the numerical value of the energy level is smaller than this number, i.e., is more negative. For example, in the present application, the expression that “the LUMO energy level of the first organic material is less than −5.1 eV” means that the numerical value of the LUMO energy level of the first organic material is more negative than −5.1, for example, the LUMO energy level of the first organic material is −5.11 eV. Herein, a difference between HOMO energy levels of an HTM and a host material is defined as HOMO_{HTM}-HOMO_{HOST}. Since the host material generally has a deeper HOMO energy level, the difference is generally positive. Herein, a difference between the HOMO energy level of the HTM and a LUMO energy level of a PD material is defined as LUMO_{PD}-HOMO_{HTM}, and the difference may be positive or negative.

Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butyldimethylgermanylmethyl, triethylgermanylmethyl, triethylgermanylethyl, triisopropylgermanylmethyl, triisopropylgermanylethyl, trimethylsilylmethyl, trimethylsilylethyl, trimethylsilylisopropyl, triisopropylsilylmethyl, and triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, propenyl, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, and phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic group or heterocycle—as used herein includes non-aromatic cyclic groups. Non-aromatic heterocyclic groups includes saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates a germanyl group substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl group may be optionally substituted.

Arylgermanyl—as used herein contemplates a germanyl group substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl group may be optionally substituted.

The term “aza” in azadibenzofuran, azadibenzothiophene, etc. means that one or more of C—H groups in the respective aromatic fragment are replaced with a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the term as set forth herein.

In the present disclosure, unless otherwise defined, when any term of the group consisting of substituted alkyl, substituted cycloalkyl, substituted heteroalkyl, a substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, a heterocyclic group, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more moieties selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl having 3 to 20 carbon atoms, unsubstituted arylgermanyl having 6 to 20 carbon atoms, unsubstituted amino having 0 to 20 carbon atoms, an acyl group, a carbonyl group, a carboxylic acid group, an ester group, a cyano group, an isocyano group, a sulfanyl group, a sulfinyl group, a sulfonyl group, a phosphino group, and combinations thereof.

It is to be understood that when a molecular fragment is described as being a substituent or otherwise attached to another moiety, its name may be written as if it was a fragment (e.g. phenyl, phenylene, naphthyl, dibenzofuryl) or as if it was the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen may also be replaced by their other stable isotopes. The replacement by other stable isotopes in the compounds may be preferred due to its enhancements of device efficiency and stability.

In the compounds mentioned in the present disclosure, multiple substitutions refer to a range that includes a di-substitution, up to the maximum available substitutions. When a substituent in the compounds mentioned in the present disclosure represents multiple substitutions (including di-, tri-, and tetra-substitutions, etc.), it means that the substituent may be present at multiple available substitution positions on the structure linked to the substituent, where substituents present at the multiple available substitution positions may have the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes the case where adjacent substituents may be joined to form a ring and the case where adjacent substituents are not joined to form a ring. When adjacent substituents can be optionally joined to form a ring, the ring formed may be monocyclic or polycyclic (including spirocyclic, endocyclic, and fusedcyclic, etc.) as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

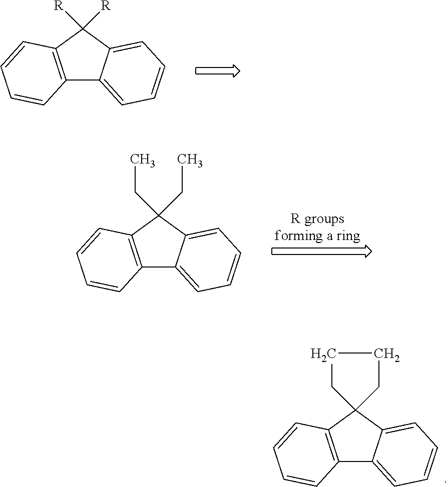

The expression that adjacent substituents can be optionally joined to form a ring is also intended to mean that two substituents bonded to carbon atoms which are directly bonded to each other are joined to each other via a chemical bond to form a ring, which can be exemplified by the following formula:

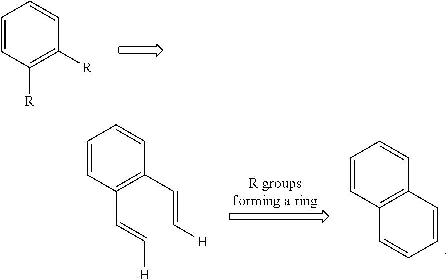

The expression that adjacent substituents can be optionally joined to form a ring is also intended to mean that two substituents bonded to further distant carbon atoms are joined to each other via a chemical bond to form a ring, which can be exemplified by the following formula:

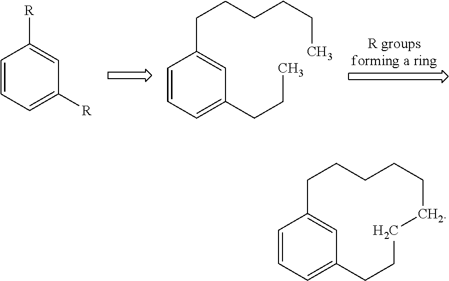

Furthermore, the expression that adjacent substituents can be optionally joined to form a ring is also intended to mean that, in the case where one of two adjacent substituents bonded to carbon atoms which are directly bonded to each other represents hydrogen, the second substituent is bonded at a position where the hydrogen atom is bonded, thereby forming a ring. This is exemplified by the following formula:

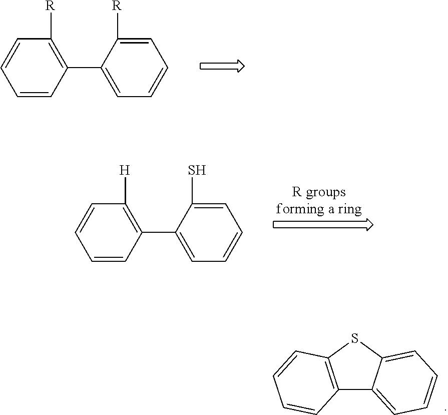

According to an embodiment of the present disclosure, an organic electroluminescent device is disclosed. The organic electroluminescent device comprises:

a substrate;

a first electrode disposed on the substrate;

a second electrode disposed over the first electrode; and

an organic layer disposed between the first electrode and the second electrode;

wherein the first electrode is a material with high reflectivity or a combination of materials with high reflectivity, and the second electrode is a translucent or transparent material or a combination of translucent or transparent materials;

the organic layer comprises a first organic layer, a second organic layer and a third organic layer;

the first organic layer comprises a first organic material and a second organic material;

the second organic layer is made of the second organic material and has a thickness of greater than 80 nm;

the third organic layer is a light-emitting layer comprising at least one light-emitting material and at least one host material;

the first organic layer has a conductivity of greater than 1×10⁻⁴ S/m and less than 1×10⁻² S/m;

an energy level difference between a HOMO energy level of the second organic material and a HOMO energy level of the at least one host material is less than 0.27 eV; and

one side of the first organic layer is in direct contact with the first electrode, and the other side of the first organic layer is in direct contact with the second organic layer.

According to an embodiment of the present disclosure, a LUMO energy level of the first organic material is less than −5.1 eV.

According to an embodiment of the present disclosure, the HOMO energy level of the second organic material is less than −5.25 eV.

According to an embodiment of the present disclosure, the second organic layer is in direct contact with the third organic layer.

According to an embodiment of the present disclosure, the first electrode is selected from the group consisting of Ag, Ti, Cr, Pt, Ni, TiN and combinations thereof with ITO and/or MoOx.

According to an embodiment of the present disclosure, the second electrode is selected from a Mg—Ag alloy, MoOx, Yb, Ca, ITO, IZO or a combination thereof.

According to an embodiment of the present disclosure, the energy level difference between the HOMO energy level of the second organic material and the HOMO energy level of the at least one host material is less than or equal to 0.26 eV.

According to an embodiment of the present disclosure, the energy level difference between the HOMO energy level of the second organic material and the HOMO energy level of the at least one host material is less than 0.25 eV.

According to an embodiment of the present disclosure, the energy level difference between the HOMO energy level of the second organic material and the HOMO energy level of the at least one host material is less than 0.2 eV.

According to an embodiment of the present disclosure, an energy level difference between the HOMO energy level of the second organic material and the LUMO energy level of the first organic material is less than 0.23 eV.

According to an embodiment of the present disclosure, the energy level difference between the HOMO energy level of the second organic material and the LUMO energy level of the first organic material is less than 0.2 eV.

According to an embodiment of the present disclosure, the energy level difference between the HOMO energy level of the second organic material and the LUMO energy level of the first organic material is less than or equal to 0.1 eV.

According to an embodiment of the present disclosure, the device further comprises an electron injection layer, where the electron injection layer is disposed between the third organic layer and the second electrode.

According to an embodiment of the present disclosure, the electron injection layer comprises the group consisting of Yb, Liq, LiF and combinations thereof.

According to an embodiment of the present disclosure, the second organic layer has a thickness of greater than or equal to 100 nm.

According to an embodiment of the present disclosure, the second organic layer has a thickness of greater than or equal to 120 nm.

According to an embodiment of the present disclosure, the second organic layer has a thickness of greater than 125 nm.

According to an embodiment of the present disclosure, the second organic layer has a thickness of greater than 150 nm.

According to an embodiment of the present disclosure, the first organic layer has a conductivity of greater than 2×10⁻⁴ S/m and less than 8×10⁻³ S/m.

According to an embodiment of the present disclosure, the first organic material has a structure represented by Formula 1:

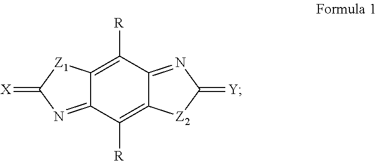

wherein in Formula 1,

X and Y are, at each occurrence identically or differently, selected from NR′, CR″R′″, O, S or Se;

Z₁ and Z₂ are, at each occurrence identically or differently, selected from O, S or Se;

R, R′, R″ and R′″ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

each R may be the same or different, and at least one of R, R′, R″ and R′″ is a group having at least one electron withdrawing group; and

in Formula 1, adjacent substituents can be optionally joined to form a ring.

According to an embodiment of the present disclosure, the second organic material has a structure represented by Formula 2:

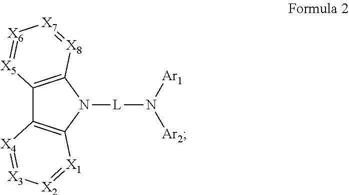

wherein in Formula 2,

X₁ to X₈ are, at each occurrence identically or differently, selected from CR₁ or N;

L is, at each occurrence identically or differently, selected from substituted or unsubstituted arylene having 6 to 30 carbon atoms, substituted or unsubstituted heteroarylene having 3 to 30 carbon atoms or a combination thereof;

Ar₁ and Ar₂ are, at each occurrence identically or differently, selected from substituted or unsubstituted aryl having 6 to 30 carbon atoms or substituted or unsubstituted heteroaryl having 3 to 30 carbon atoms;

R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

in Formula 2, adjacent substituents can be optionally joined to form a ring.

According to an embodiment of the present disclosure, in Formula 1, X and Y are, at each occurrence identically or differently, selected from CR″R′″ or NR′, and at least one of R′, R″ and R′″ is/are a group having at least one electron withdrawing group; preferably, R, R′, R″ and R′″ each are a group having at least one electron withdrawing group.

According to an embodiment of the present disclosure, in Formula 1, X and Y are, at each occurrence identically or differently, selected from O, S or Se, and at least one R is a group having at least one electron withdrawing group; preferably, each R is a group having at least one electron withdrawing group.

According to an embodiment of the present disclosure, in Formula 1, a Hammett constant of the electron withdrawing group is ≥0.05, preferably ≥0.3, and more preferably ≥0.5.

According to an embodiment of the present disclosure, in Formula 1, the electron withdrawing group is selected from the group consisting of: halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, an aza-aromatic ring group and any one of the following groups substituted by one or more of halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group and an aza-aromatic ring group: alkyl having 1 to 20 carbon atoms, cycloalkyl having 3 to 20 ring carbon atoms, heteroalkyl having 1 to 20 carbon atoms, arylalkyl having 7 to 30 carbon atoms, alkoxy having 1 to 20 carbon atoms, aryloxy having 6 to 30 carbon atoms, alkenyl having 2 to 20 carbon atoms, alkynyl having 2 to 20 carbon atoms, aryl having 6 to 30 carbon atoms, heteroaryl having 3 to 30 carbon atoms, alkylsilyl having 3 to 20 carbon atoms, arylsilyl having 6 to 20 carbon atoms and combinations thereof.

According to an embodiment of the present disclosure, in Formula 1, the electron withdrawing group is selected from the group consisting of: F, CF₃, OCF₃, SF₅, SO₂CF₃, cyano, isocyano, SCN, OCN, pyrimidinyl, triazinyl and combinations thereof.

According to an embodiment of the present disclosure, in Formula 1, X and Y are, at each occurrence identically or differently, selected from the group consisting of the following structures:

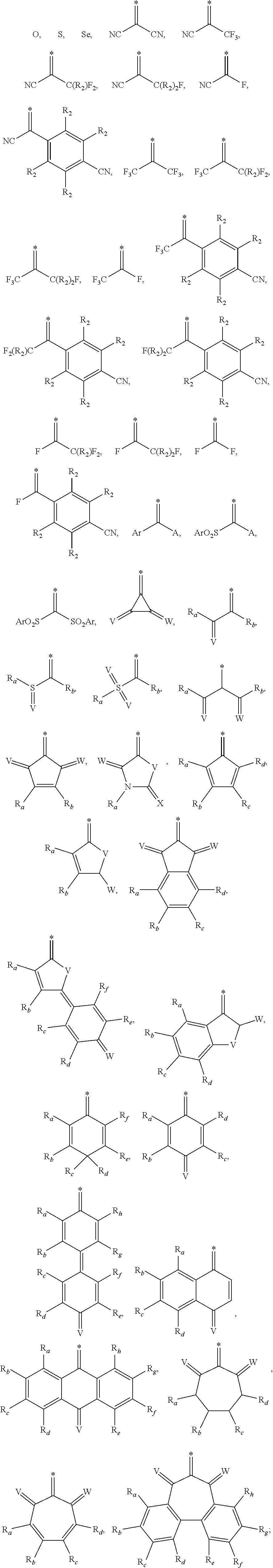

wherein R₂ is, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof;

preferably, R₂ is, at each occurrence identically or differently, selected from the group consisting of: F, CF₃, OCF₃, SF₅, SO₂CF₃, cyano, isocyano, SCN, OCN, pentafluorophenyl, 4-cyanotetrafluorophenyl, tetrafluoropyridyl, pyrimidinyl, triazinyl and combinations thereof;

wherein V and W are, at each occurrence identically or differently, selected from CRᵥR_{w}, NR, O, S or Se;

wherein Ar is, at each occurrence identically or differently, selected from substituted or unsubstituted aryl having 6 to 30 carbon atoms or substituted or unsubstituted heteroaryl having 3 to 30 carbon atoms;

wherein A, Rₐ, R_{b}, Rₑ, R_{d}, Rₑ, R_{f}, R_{g}, Rₕ, Rᵥ and R_{w} are, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof;

wherein A is a group having at least one electron withdrawing group, and for any one of the structures, when one or more of Rₐ, R_{b}, R_{c}, R_{d}, Rₑ, R_{f}, R_{g}, Rₕ, Rᵥ and R_{w} are present, at least one of Rₐ, R_{b}, R_{c}, R_{d}, Rₑ, R_{f}, R_{g}, Rₕ, Rᵥ and R_{w} is a group having at least one electron withdrawing group; preferably, the group having at least one electron withdrawing group is selected from the group consisting of: F, CF₃, OCF₃, SF₅, SO₂CF₃, cyano, isocyano, SCN, OCN, pentafluorophenyl, 4-cyanotetrafluorophenyl, tetrafluoropyridyl, pyrimidinyl, triazinyl and combinations thereof; and

wherein “*” represents a position where X and Y are joined to a dehydrobenzodioxazole ring, a dehydrobenzodithiazole ring or a dehydrobenzodiselenazole ring in Formula 1.

According to an embodiment of the present disclosure, in Formula 1, X and Y are, at each occurrence identically or differently, selected from the group consisting of the following structures:

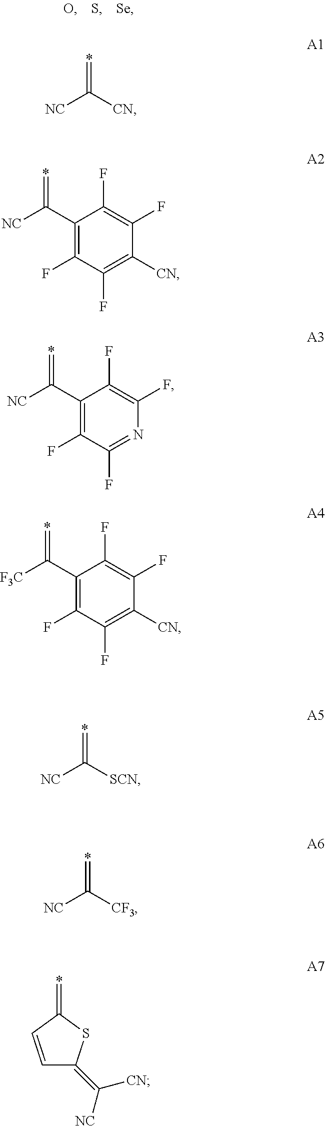

wherein “*” represents a position where X or Y is joined to a dehydrobenzodioxazole ring, a dehydrobenzodithiazole ring or a dehydrobenzodiselenazole ring in Formula 1.

According to an embodiment of the present disclosure, in Formula 1, R is, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, unsubstituted alkyl having 1 to 20 carbon atoms, unsubstituted cycloalkyl having 3 to 20 ring carbon atoms, unsubstituted alkoxy having 1 to 20 carbon atoms, unsubstituted alkenyl having 2 to 20 carbon atoms, unsubstituted aryl having 6 to 30 carbon atoms, unsubstituted heteroaryl having 3 to 30 carbon atoms and any one of the following groups substituted by one or more of halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group and a phosphoroso group: alkyl having 1 to 20 carbon atoms, cycloalkyl having 3 to 20 ring carbon atoms, alkoxy having 1 to 20 carbon atoms, alkenyl having 2 to 20 carbon atoms, aryl having 6 to 30 carbon atoms, heteroaryl having 3 to 30 carbon atoms and combinations thereof.

According to an embodiment of the present disclosure, in Formula 1, R is, at each occurrence identically or differently, selected from the group consisting of: hydrogen, deuterium, methyl, isopropyl, NO₂, SO₂CH₃, SCF₃, C₂F₅, OC₂F₅, OCH₃, diphenylmethylsilyl, phenyl, methoxyphenyl, p-methylphenyl, 2,6-diisopropylphenyl, biphenyl, polyfluorophenyl, difluoropyridyl, nitrophenyl, dimethylthiazolyl, vinyl substituted by one or more of CN or CF₃, acetenyl substituted by one of CN or CF₃, dimethylphosphoroso, diphenylphosphoroso, F, CF₃, OCF₃, SF₅, SO₂CF₃, cyano, isocyano, SCN, OCN, trifluoromethylphenyl, trifluoromethoxyphenyl, bis(trifluoromethyl)phenyl, bis(trifluoromethoxy)phenyl, 4-cyanotetrafluorophenyl, phenyl or biphenyl substituted by one or more of F, CN or CF₃, tetrafluoropyridyl, pyrimidinyl, triazinyl, diphenylboryl, oxaboraanthryl and combinations thereof.

According to an embodiment of the present disclosure, in Formula 1, X and Y each are

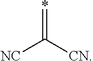 [SMILES: *=C(C#N)C#N]

According to an embodiment of the present disclosure, in Formula 1, R is, at each occurrence identically or differently, selected from the group consisting of the following structures:

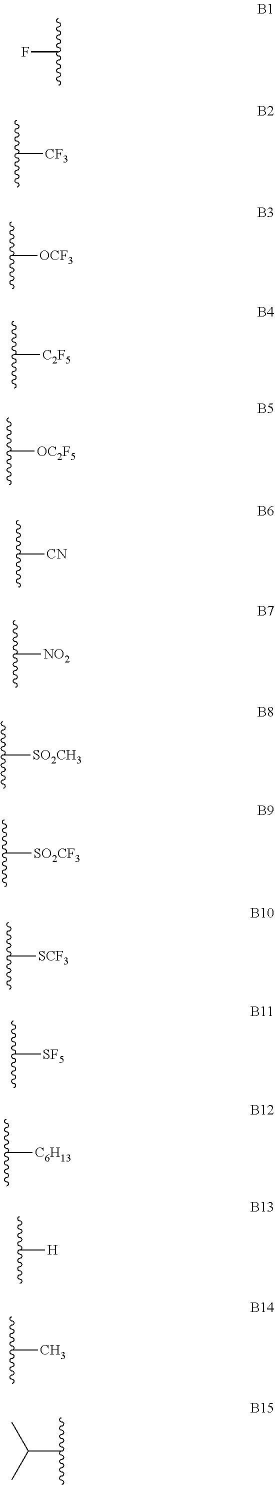

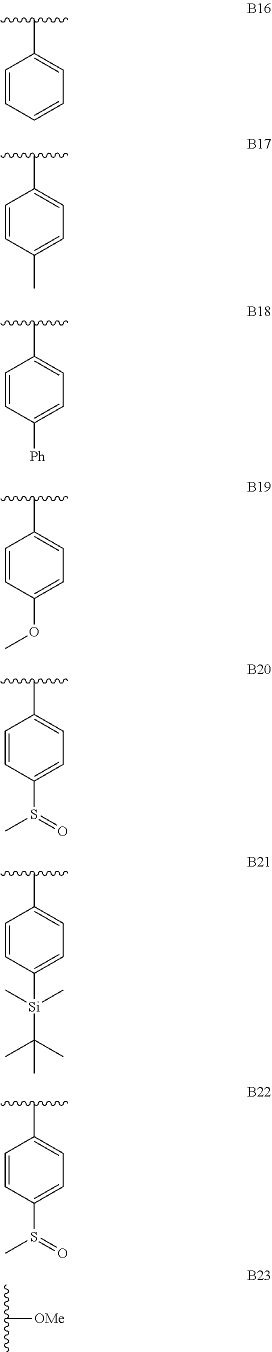

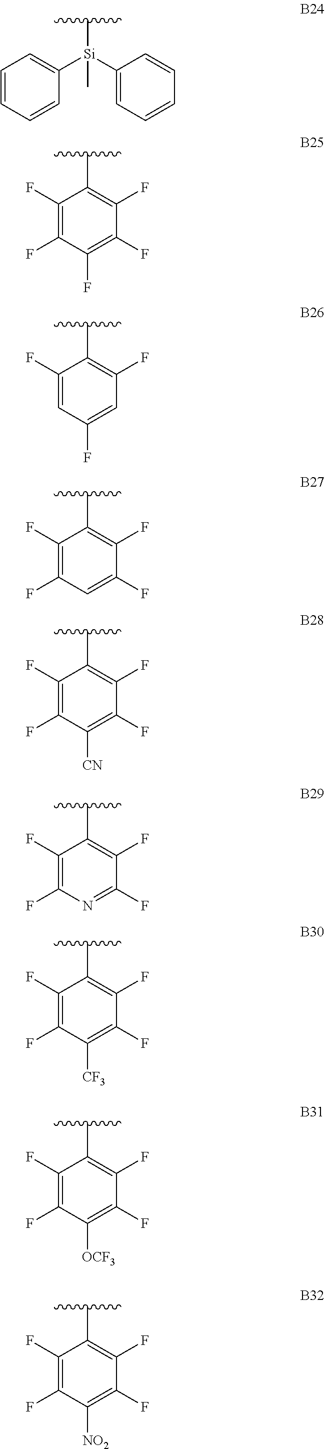

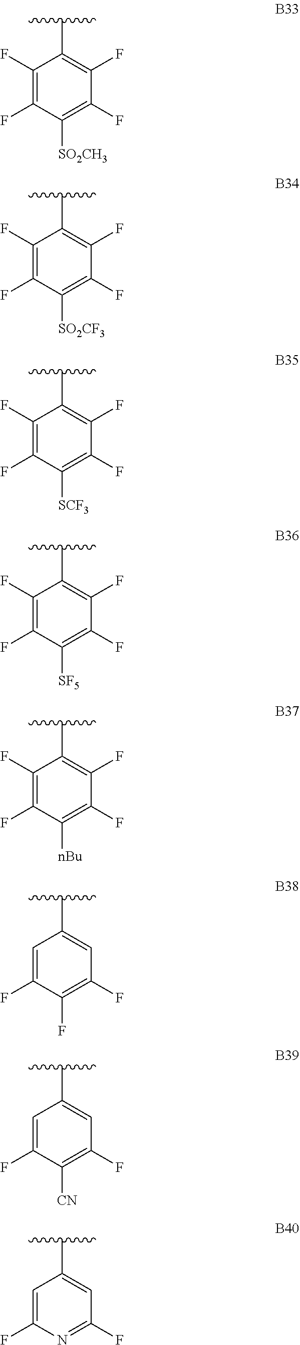

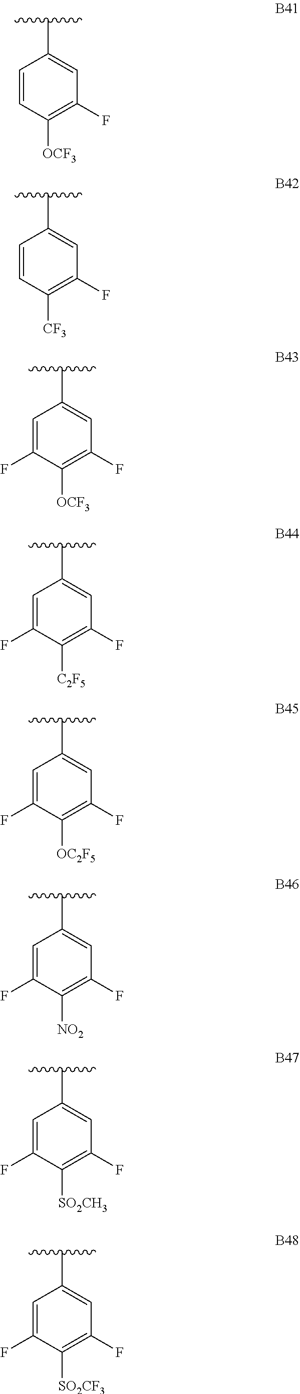

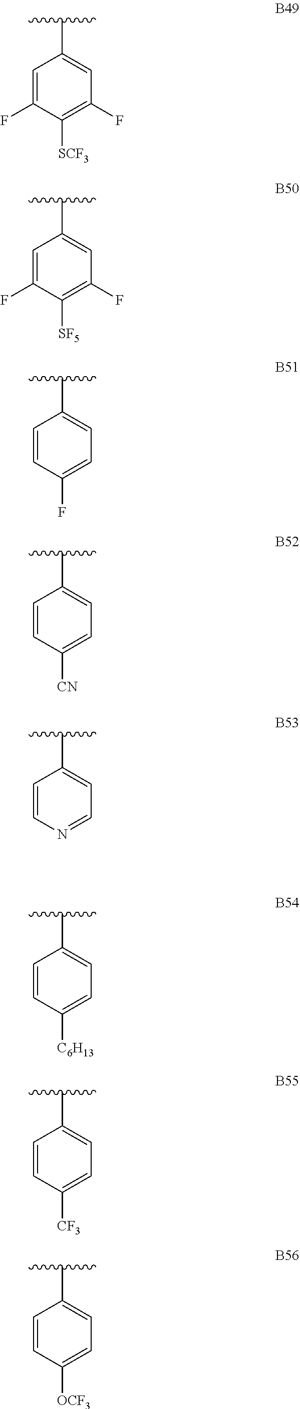

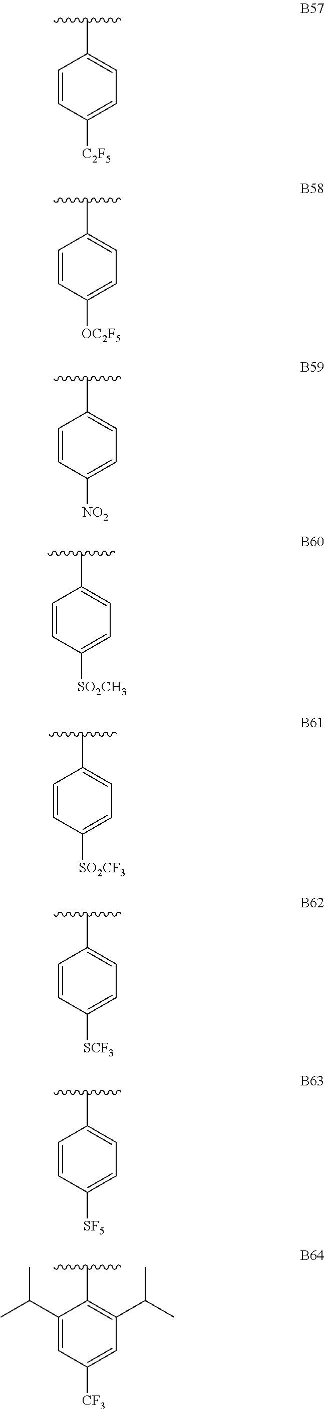

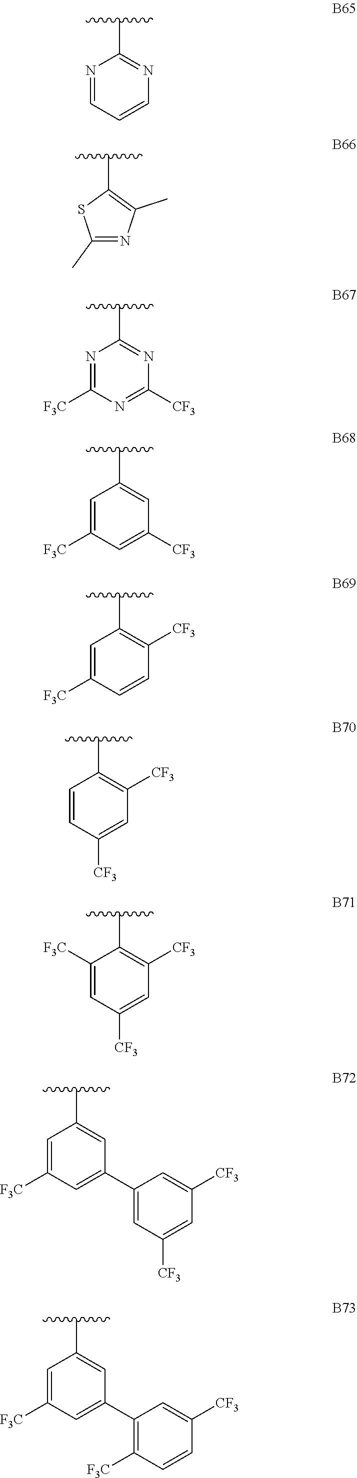

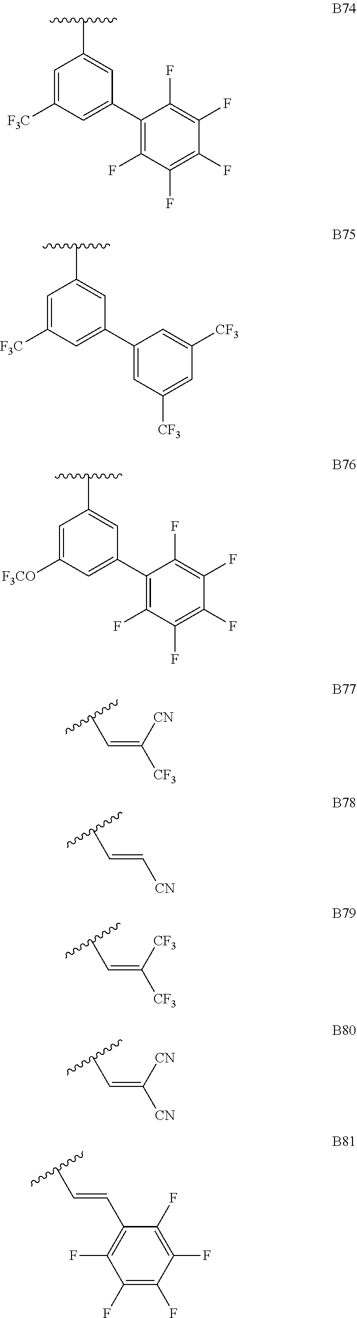

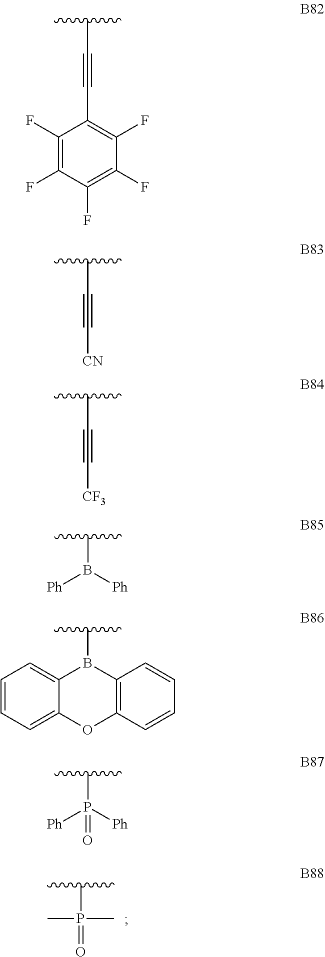

wherein “ [symbol] ” represents a position where the group R is joined to a dehydrobenzodioxazole ring, a dehydrobenzodithiazole ring or a dehydrobenzodiselenazole in Formula 1.

According to an embodiment of the present disclosure, two R in one compound represented by Formula 1 are the same.

According to an embodiment of the present disclosure, the compound of Formula 1 has a structure represented by Formula 3:

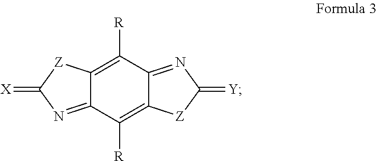

wherein in Formula 3, two Z have the same structure, two R have the same structure or different structures, and Z, X, Y and R are respectively and correspondingly selected from atoms or groups shown in the following table;

wherein the compound having the structure of Formula 3 is selected from the group consisting of the following compounds:

| No. | Z | X | Y | R | R | No. | Z | X | Y | R | R |
|---|---|---|---|---|---|---|---|---|---|---|---|
| Compound 1 | O | A1 | A1 | B1 | B1 | Compound 2 | O | A1 | A1 | B2 | B2 |
| Compound 3 | O | A1 | A1 | B3 | B3 | Compound 4 | O | A1 | A1 | B4 | B4 |
| Compound 5 | O | A1 | A1 | B5 | B5 | Compound 6 | O | A1 | A1 | B6 | B6 |
| Compound 7 | O | A1 | A1 | B7 | B7 | Compound 8 | O | A1 | A1 | B8 | B8 |
| Compound 9 | O | A1 | A1 | B9 | B9 | Compound 10 | O | A1 | A1 | B10 | B10 |
| Compound 11 | O | A1 | A1 | B11 | B11 | Compound 12 | O | A1 | A1 | B12 | B12 |
| Compound 13 | O | A1 | A1 | B13 | B13 | Compound 14 | O | A1 | A1 | B14 | B14 |
| Compound 15 | O | A1 | A1 | B15 | B15 | Compound 16 | O | A1 | A1 | B16 | B16 |
| Compound 17 | O | A1 | A1 | B17 | B17 | Compound 18 | O | A1 | A1 | B18 | B18 |
| Compound 19 | O | A1 | A1 | B19 | B19 | Compound 20 | O | A1 | A1 | B20 | B20 |
| Compound 21 | O | A1 | A1 | B21 | B21 | Compound 22 | O | A1 | A1 | B22 | B22 |
| Compound 23 | O | A1 | A1 | B23 | B23 | Compound 24 | O | A1 | A1 | B24 | B24 |
| Compound 25 | O | A1 | A1 | B25 | B25 | Compound 26 | O | A1 | A1 | B26 | B26 |
| Compound 27 | O | A1 | A1 | B27 | B27 | Compound 28 | O | A1 | A1 | B28 | B28 |
| Compound 29 | O | A1 | A1 | B29 | B29 | Compound 30 | O | A1 | A1 | B30 | B30 |
| Compound 31 | O | A1 | A1 | B31 | B31 | Compound 32 | O | A1 | A1 | B32 | B32 |
| Compound 33 | O | A1 | A1 | B33 | B33 | Compound 34 | O | A1 | A1 | B34 | B34 |
| Compound 35 | O | A1 | A1 | B35 | B35 | Compound 36 | O | A1 | A1 | B36 | B36 |
| Compound 37 | O | A1 | A1 | B37 | B37 | Compound 38 | O | A1 | A1 | B38 | B38 |
| Compound 39 | O | A1 | A1 | B39 | B39 | Compound 40 | O | A1 | A1 | B40 | B40 |
| Compound 41 | O | A1 | A1 | B41 | B41 | Compound 42 | O | A1 | A1 | B42 | B42 |
| Compound 43 | O | A1 | A1 | B43 | B43 | Compound 44 | O | A1 | A1 | B44 | B44 |
| Compound 45 | O | A1 | A1 | B45 | B45 | Compound 46 | O | A1 | A1 | B46 | B46 |
| Compound 47 | O | A1 | A1 | B47 | B47 | Compound 48 | O | A1 | A1 | B48 | B48 |
| Compound 49 | O | A1 | A1 | B49 | B49 | Compound 50 | O | A1 | A1 | B50 | B50 |
| Compound 51 | O | A1 | A1 | B51 | B51 | Compound 52 | O | A1 | A1 | B52 | B52 |
| Compound 53 | O | A1 | A1 | B53 | B53 | Compound 54 | O | A1 | A1 | B54 | B54 |
| Compound 55 | O | A1 | A1 | B55 | B55 | Compound 56 | O | A1 | A1 | B56 | B56 |
| Compound 57 | O | A1 | A1 | B57 | B57 | Compound 58 | O | A1 | A1 | B58 | B58 |
| Compound 59 | O | A1 | A1 | B59 | B59 | Compound 60 | O | A1 | A1 | B60 | B60 |
| Compound 61 | O | A1 | A1 | B61 | B61 | Compound 62 | O | A1 | A1 | B62 | B62 |
| Compound 63 | O | A1 | A1 | B63 | B63 | Compound 64 | O | A1 | A1 | B64 | B64 |
| Compound 65 | O | A1 | A1 | B65 | B65 | Compound 66 | O | A1 | A1 | B66 | B66 |
| Compound 67 | O | A1 | A1 | B67 | B67 | Compound 68 | O | A1 | A1 | B68 | B68 |
| Compound 69 | O | A1 | A1 | B69 | B69 | Compound 70 | O | A1 | A1 | B70 | B70 |
| Compound 71 | O | A1 | A1 | B71 | B71 | Compound 72 | O | A1 | A1 | B72 | B72 |
| Compound 73 | O | A1 | A1 | B73 | B73 | Compound 74 | O | A1 | A1 | B74 | B74 |
| Compound 75 | O | A1 | A1 | B75 | B75 | Compound 76 | O | A1 | A1 | B76 | B76 |
| Compound 77 | O | A1 | A1 | B77 | B77 | Compound 78 | O | A1 | A1 | B78 | B78 |
| Compound 79 | O | A1 | A1 | B79 | B79 | Compound 80 | O | A1 | A1 | B80 | B80 |
| Compound 81 | O | A1 | A1 | B81 | B81 | Compound 82 | O | A1 | A1 | B82 | B82 |
| Compound 83 | O | A1 | A1 | B83 | B83 | Compound 84 | O | A1 | A1 | B84 | B84 |
| Compound 85 | O | A1 | A1 | B85 | B85 | Compound 86 | O | A1 | A1 | B86 | B86 |
| Compound 87 | O | A1 | A1 | B87 | B87 | Compound 88 | O | A1 | A1 | B88 | B88 |
| Compound 89 | S | A1 | A1 | B1 | B1 | Compound 90 | S | A1 | A1 | B2 | B2 |
| Compound 91 | S | A1 | A1 | B3 | B3 | Compound 92 | S | A1 | A1 | B4 | B4 |
| Compound 93 | S | A1 | A1 | B5 | B5 | Compound 94 | S | A1 | A1 | B6 | B6 |
| Compound 95 | S | A1 | A1 | B7 | B7 | Compound 96 | S | A1 | A1 | B8 | B8 |
| Compound 97 | S | A1 | A1 | B9 | B9 | Compound 98 | S | A1 | A1 | B10 | B10 |
| Compound 99 | S | A1 | A1 | B11 | B11 | Compound 100 | S | A1 | A1 | B12 | B12 |
| Compound 101 | S | A1 | A1 | B13 | B13 | Compound 102 | S | A1 | A1 | B14 | B14 |
| Compound 103 | S | A1 | A1 | B15 | B15 | Compound 104 | S | A1 | A1 | B16 | B16 |
| Compound 105 | S | A1 | A1 | B17 | B17 | Compound 106 | S | A1 | A1 | B18 | B18 |
| Compound 107 | S | A1 | A1 | B19 | B19 | Compound 108 | S | A1 | A1 | B20 | B20 |
| Compound 109 | S | A1 | A1 | B21 | B21 | Compound 110 | S | A1 | A1 | B22 | B22 |
| Compound 111 | S | A1 | A1 | B23 | B23 | Compound 112 | S | A1 | A1 | B24 | B24 |
| Compound 113 | S | A1 | A1 | B25 | B25 | Compound 114 | S | A1 | A1 | B26 | B26 |
| Compound 115 | S | A1 | A1 | B27 | B27 | Compound 116 | S | A1 | A1 | B28 | B28 |
| Compound 117 | S | A1 | A1 | B29 | B29 | Compound 118 | S | A1 | A1 | B30 | B30 |
| Compound 119 | S | A1 | A1 | B31 | B31 | Compound 120 | S | A1 | A1 | B32 | B32 |
| Compound 121 | S | A1 | A1 | B33 | B33 | Compound 122 | S | A1 | A1 | B34 | B34 |
| Compound 123 | S | A1 | A1 | B35 | B35 | Compound 124 | S | A1 | A1 | B36 | B36 |
| Compound 125 | S | A1 | A1 | B37 | B37 | Compound 126 | S | A1 | A1 | B38 | B38 |
| Compound 127 | S | A1 | A1 | B39 | B39 | Compound 128 | S | A1 | A1 | B40 | B40 |
| Compound 129 | S | A1 | A1 | B41 | B41 | Compound 130 | S | A1 | A1 | B42 | B42 |
| Compound 131 | S | A1 | A1 | B43 | B43 | Compound 132 | S | A1 | A1 | B44 | B44 |
| Compound 133 | S | A1 | A1 | B45 | B45 | Compound 134 | S | A1 | A1 | B46 | B46 |
| Compound 135 | S | A1 | A1 | B47 | B47 | Compound 136 | S | A1 | A1 | B48 | B48 |
| Compound 137 | S | A1 | A1 | B49 | B49 | Compound 138 | S | A1 | A1 | B50 | B50 |
| Compound 139 | S | A1 | A1 | B51 | B51 | Compound 140 | S | A1 | A1 | B52 | B52 |
| Compound 141 | S | A1 | A1 | B53 | B53 | Compound 142 | S | A1 | A1 | B54 | B54 |
| Compound 143 | S | A1 | A1 | B55 | B55 | Compound 144 | S | A1 | A1 | B56 | B56 |
| Compound 145 | S | A1 | A1 | B57 | B57 | Compound 146 | S | A1 | A1 | B58 | B58 |
| Compound 147 | S | A1 | A1 | B59 | B59 | Compound 148 | S | A1 | A1 | B60 | B60 |
| Compound 149 | S | A1 | A1 | B61 | B61 | Compound 150 | S | A1 | A1 | B62 | B62 |
| Compound 151 | S | A1 | A1 | B63 | B63 | Compound 152 | S | A1 | A1 | B64 | B64 |
| Compound 153 | S | A1 | A1 | B65 | B65 | Compound 154 | S | A1 | A1 | B66 | B66 |
| Compound 155 | S | A1 | A1 | B67 | B67 | Compound 156 | S | A1 | A1 | B68 | B68 |
| Compound 157 | S | A1 | A1 | B69 | B69 | Compound 158 | S | A1 | A1 | B70 | B70 |
| Compound 159 | S | A1 | A1 | B71 | B71 | Compound 160 | S | A1 | A1 | B72 | B72 |
| Compound 161 | S | A1 | A1 | B73 | B73 | Compound 162 | S | A1 | A1 | B74 | B74 |
| Compound 163 | S | A1 | A1 | B75 | B75 | Compound 164 | S | A1 | A1 | B76 | B76 |
| Compound 165 | S | A1 | A1 | B77 | B77 | Compound 166 | S | A1 | A1 | B78 | B78 |
| Compound 167 | S | A1 | A1 | B79 | B79 | Compound 168 | S | A1 | A1 | B80 | B80 |
| Compound 169 | S | A1 | A1 | B81 | B81 | Compound 170 | S | A1 | A1 | B82 | B82 |
| Compound 171 | S | A1 | A1 | B83 | B83 | Compound 172 | S | A1 | A1 | B84 | B84 |
| Compound 173 | S | A1 | A1 | B85 | B85 | Compound 174 | S | A1 | A1 | B86 | B86 |
| Compound 175 | S | A1 | A1 | B87 | B87 | Compound 176 | S | A1 | A1 | B88 | B88 |
| Compound 177 | Se | A1 | A1 | B1 | B1 | Compound 178 | Se | A1 | A1 | B2 | B2 |
| Compound 179 | Se | A1 | A1 | B3 | B3 | Compound 180 | Se | A1 | A1 | B4 | B4 |
| Compound 181 | Se | A1 | A1 | B5 | B5 | Compound 182 | Se | A1 | A1 | B6 | B6 |
| Compound 183 | Se | A1 | A1 | B7 | B7 | Compound 184 | Se | A1 | A1 | B8 | B8 |
| Compound 185 | Se | A1 | A1 | B9 | B9 | Compound 186 | Se | A1 | A1 | B10 | B10 |
| Compound 187 | Se | A1 | A1 | B11 | B11 | Compound 188 | Se | A1 | A1 | B12 | B12 |
| Compound 189 | Se | A1 | A1 | B13 | B13 | Compound 190 | Se | A1 | A1 | B14 | B14 |
| Compound 191 | Se | A1 | A1 | B15 | B15 | Compound 192 | Se | A1 | A1 | B16 | B16 |
| Compound 193 | Se | A1 | A1 | B17 | B17 | Compound 194 | Se | A1 | A1 | B18 | B18 |
| Compound 195 | Se | A1 | A1 | B19 | B19 | Compound 196 | Se | A1 | A1 | B20 | B20 |
| Compound 197 | Se | A1 | A1 | B21 | B21 | Compound 198 | Se | A1 | A1 | B22 | B22 |
| Compound 199 | Se | A1 | A1 | B23 | B23 | Compound 200 | Se | A1 | A1 | B24 | B24 |
| Compound 201 | Se | A1 | A1 | B25 | B25 | Compound 202 | Se | A1 | A1 | B26 | B26 |
| Compound 203 | Se | A1 | A1 | B27 | B27 | Compound 204 | Se | A1 | A1 | B28 | B28 |
| Compound 205 | Se | A1 | A1 | B29 | B29 | Compound 206 | Se | A1 | A1 | B30 | B30 |
| Compound 207 | Se | A1 | A1 | B31 | B31 | Compound 208 | Se | A1 | A1 | B32 | B32 |
| Compound 209 | Se | A1 | A1 | B33 | B33 | Compound 210 | Se | A1 | A1 | B34 | B34 |
| Compound 211 | Se | A1 | A1 | B35 | B35 | Compound 212 | Se | A1 | A1 | B36 | B36 |
| Compound 213 | Se | A1 | A1 | B37 | B37 | Compound 214 | Se | A1 | A1 | B38 | B38 |
| Compound 215 | Se | A1 | A1 | B39 | B39 | Compound 216 | Se | A1 | A1 | B40 | B40 |
| Compound 217 | Se | A1 | A1 | B41 | B41 | Compound 218 | Se | A1 | A1 | B42 | B42 |
| Compound 219 | Se | A1 | A1 | B43 | B43 | Compound 220 | Se | A1 | A1 | B44 | B44 |
| Compound 221 | Se | A1 | A1 | B45 | B45 | Compound 222 | Se | A1 | A1 | B46 | B46 |
| Compound 223 | Se | A1 | A1 | B47 | B47 | Compound 224 | Se | A1 | A1 | B48 | B48 |
| Compound 225 | Se | A1 | A1 | B49 | B49 | Compound 226 | Se | A1 | A1 | B50 | B50 |
| Compound 227 | Se | A1 | A1 | B51 | B51 | Compound 228 | Se | A1 | A1 | B52 | B52 |
| Compound 229 | Se | A1 | A1 | B53 | B53 | Compound 230 | Se | A1 | A1 | B54 | B54 |
| Compound 231 | Se | A1 | A1 | B55 | B55 | Compound 232 | Se | A1 | A1 | B56 | B56 |
| Compound 233 | Se | A1 | A1 | B57 | B57 | Compound 234 | Se | A1 | A1 | B58 | B58 |
| Compound 235 | Se | A1 | A1 | B59 | B59 | Compound 236 | Se | A1 | A1 | B60 | B60 |
| Compound 237 | Se | A1 | A1 | B61 | B61 | Compound 238 | Se | A1 | A1 | B62 | B62 |
| Compound 239 | Se | A1 | A1 | B63 | B63 | Compound 240 | Se | A1 | A1 | B64 | B64 |
| Compound 241 | Se | A1 | A1 | B65 | B65 | Compound 242 | Se | A1 | A1 | B66 | B66 |
| Compound 243 | Se | A1 | A1 | B67 | B67 | Compound 244 | Se | A1 | A1 | B68 | B68 |
| Compound 245 | Se | A1 | A1 | B69 | B69 | Compound 246 | Se | A1 | A1 | B70 | B70 |
| Compound 247 | Se | A1 | A1 | B71 | B71 | Compound 248 | Se | A1 | A1 | B72 | B72 |
| Compound 249 | Se | A1 | A1 | B73 | B73 | Compound 250 | Se | A1 | A1 | B74 | B74 |
| Compound 251 | Se | A1 | A1 | B75 | B75 | Compound 252 | Se | A1 | A1 | B76 | B76 |
| Compound 253 | Se | A1 | A1 | B77 | B77 | Compound 254 | Se | A1 | A1 | B78 | B78 |
| Compound 255 | Se | A1 | A1 | B79 | B79 | Compound 256 | Se | A1 | A1 | B80 | B80 |
| Compound 257 | Se | A1 | A1 | B81 | B81 | Compound 258 | Se | A1 | A1 | B82 | B82 |
| Compound 259 | Se | A1 | A1 | B83 | B83 | Compound 260 | Se | A1 | A1 | B84 | B84 |
| Compound 261 | Se | A1 | A1 | B85 | B85 | Compound 262 | Se | A1 | A1 | B86 | B86 |
| Compound 263 | Se | A1 | A1 | B87 | B87 | Compound 264 | Se | A1 | A1 | B88 | B88 |
| Compound 265 | O | A2 | A2 | B1 | B1 | Compound 266 | O | A2 | A2 | B6 | B6 |
| Compound 267 | O | A2 | A2 | B10 | B10 | Compound 268 | O | A2 | A2 | B16 | B16 |
| Compound 269 | O | A2 | A2 | B25 | B25 | Compound 270 | O | A2 | A2 | B28 | B28 |
| Compound 271 | O | A2 | A2 | B29 | B29 | Compound 272 | O | A2 | A2 | B30 | B30 |
| Compound 273 | O | A2 | A2 | B38 | B38 | Compound 274 | O | A2 | A2 | B39 | B39 |
| Compound 275 | O | A2 | A2 | B40 | B40 | Compound 276 | O | A2 | A2 | B41 | B41 |
| Compound 277 | O | A2 | A2 | B43 | B43 | Compound 278 | O | A2 | A2 | B52 | B52 |
| Compound 279 | O | A2 | A2 | B56 | B56 | Compound 280 | O | A2 | A2 | B67 | B67 |
| Compound 281 | O | A2 | A2 | B68 | B68 | Compound 282 | O | A2 | A2 | B69 | B69 |
| Compound 283 | O | A2 | A2 | B70 | B70 | Compound 284 | O | A2 | A2 | B71 | B71 |
| Compound 285 | O | A2 | A2 | B72 | B72 | Compound 286 | O | A2 | A2 | B74 | B74 |
| Compound 287 | O | A2 | A2 | B79 | B79 | Compound 288 | O | A2 | A2 | B80 | B80 |
| Compound 289 | O | A2 | A2 | B82 | B82 | Compound 290 | O | A2 | A2 | B83 | B83 |
| Compound 291 | O | A2 | A2 | B86 | B86 | Compound 292 | O | A2 | A2 | B88 | B88 |
| Compound 293 | S | A2 | A2 | B1 | B1 | Compound 294 | S | A2 | A2 | B6 | B6 |
| Compound 295 | S | A2 | A2 | B10 | B10 | Compound 296 | S | A2 | A2 | B16 | B16 |
| Compound 297 | S | A2 | A2 | B25 | B25 | Compound 298 | S | A2 | A2 | B28 | B28 |
| Compound 299 | S | A2 | A2 | B29 | B29 | Compound 300 | S | A2 | A2 | B30 | B30 |
| Compound 301 | S | A2 | A2 | B38 | B38 | Compound 302 | S | A2 | A2 | B39 | B39 |
| Compound 303 | S | A2 | A2 | B40 | B40 | Compound 304 | S | A2 | A2 | B41 | B41 |
| Compound 305 | S | A2 | A2 | B43 | B43 | Compound 306 | S | A2 | A2 | B52 | B52 |
| Compound 307 | S | A2 | A2 | B56 | B56 | Compound 308 | S | A2 | A2 | B67 | B67 |
| Compound 309 | S | A2 | A2 | B68 | B68 | Compound 310 | S | A2 | A2 | B69 | B69 |
| Compound 311 | S | A2 | A2 | B70 | B70 | Compound 312 | S | A2 | A2 | B71 | B71 |
| Compound 313 | S | A2 | A2 | B72 | B72 | Compound 314 | S | A2 | A2 | B74 | B74 |
| Compound 315 | S | A2 | A2 | B79 | B79 | Compound 316 | S | A2 | A2 | B80 | B80 |
| Compound 317 | S | A2 | A2 | B82 | B82 | Compound 318 | S | A2 | A2 | B83 | B83 |
| Compound 319 | S | A2 | A2 | B86 | B86 | Compound 320 | S | A2 | A2 | B88 | B88 |
| Compound 321 | Se | A2 | A2 | B1 | B1 | Compound 322 | Se | A2 | A2 | B6 | B6 |
| Compound 323 | Se | A2 | A2 | B10 | B10 | Compound 324 | Se | A2 | A2 | B16 | B16 |
| Compound 325 | Se | A2 | A2 | B25 | B25 | Compound 326 | Se | A2 | A2 | B28 | B28 |
| Compound 327 | Se | A2 | A2 | B29 | B29 | Compound 328 | Se | A2 | A2 | B30 | B30 |
| Compound 329 | Se | A2 | A2 | B38 | B38 | Compound 330 | Se | A2 | A2 | B39 | B39 |
| Compound 331 | Se | A2 | A2 | B40 | B40 | Compound 332 | Se | A2 | A2 | B41 | B41 |
| Compound 333 | Se | A2 | A2 | B43 | B43 | Compound 334 | Se | A2 | A2 | B52 | B52 |
| Compound 335 | Se | A2 | A2 | B56 | B56 | Compound 336 | Se | A2 | A2 | B67 | B67 |
| Compound 337 | Se | A2 | A2 | B68 | B68 | Compound 338 | Se | A2 | A2 | B69 | B69 |
| Compound 339 | Se | A2 | A2 | B70 | B70 | Compound 340 | Se | A2 | A2 | B71 | B71 |
| Compound 341 | Se | A2 | A2 | B72 | B72 | Compound 342 | Se | A2 | A2 | B74 | B74 |
| Compound 343 | Se | A2 | A2 | B79 | B79 | Compound 344 | Se | A2 | A2 | B80 | B80 |
| Compound 345 | Se | A2 | A2 | B82 | B82 | Compound 346 | Se | A2 | A2 | B83 | B83 |
| Compound 347 | Se | A2 | A2 | B86 | B86 | Compound 348 | Se | A2 | A2 | B88 | B88 |
| Compound 349 | O | A3 | A3 | B1 | B1 | Compound 350 | O | A3 | A3 | B6 | B6 |
| Compound 351 | O | A3 | A3 | B10 | B10 | Compound 352 | O | A3 | A3 | B16 | B16 |
| Compound 353 | O | A3 | A3 | B25 | B25 | Compound 354 | O | A3 | A3 | B28 | B28 |
| Compound 355 | O | A3 | A3 | B29 | B29 | Compound 356 | O | A3 | A3 | B30 | B30 |
| Compound 357 | O | A3 | A3 | B38 | B38 | Compound 358 | O | A3 | A3 | B39 | B39 |
| Compound 359 | O | A3 | A3 | B40 | B40 | Compound 360 | O | A3 | A3 | B41 | B41 |
| Compound 361 | O | A3 | A3 | B43 | B43 | Compound 362 | O | A3 | A3 | B52 | B52 |
| Compound 363 | O | A3 | A3 | B56 | B56 | Compound 364 | O | A3 | A3 | B67 | B67 |
| Compound 365 | O | A3 | A3 | B68 | B68 | Compound 366 | O | A3 | A3 | B69 | B69 |
| Compound 367 | O | A3 | A3 | B70 | B70 | Compound 368 | O | A3 | A3 | B71 | B71 |
| Compound 369 | O | A3 | A3 | B72 | B72 | Compound 370 | O | A3 | A3 | B74 | B74 |
| Compound 371 | O | A3 | A3 | B79 | B79 | Compound 372 | O | A3 | A3 | B80 | B80 |
| Compound 373 | O | A3 | A3 | B82 | B82 | Compound 374 | O | A3 | A3 | B83 | B83 |
| Compound 375 | O | A3 | A3 | B86 | B86 | Compound 376 | O | A3 | A3 | B88 | B88 |
| Compound 377 | S | A3 | A3 | B1 | B1 | Compound 378 | S | A3 | A3 | B6 | B6 |
| Compound 379 | S | A3 | A3 | B10 | B10 | Compound 380 | S | A3 | A3 | B16 | B16 |
| Compound 381 | S | A3 | A3 | B25 | B25 | Compound 382 | S | A3 | A3 | B28 | B28 |
| Compound 383 | S | A3 | A3 | B29 | B29 | Compound 384 | S | A3 | A3 | B30 | B30 |
| Compound 385 | S | A3 | A3 | B38 | B38 | Compound 386 | S | A3 | A3 | B39 | B39 |
| Compound 387 | S | A3 | A3 | B40 | B40 | Compound 388 | S | A3 | A3 | B41 | B41 |
| Compound 389 | S | A3 | A3 | B43 | B43 | Compound 390 | S | A3 | A3 | B52 | B52 |
| Compound 391 | S | A3 | A3 | B56 | B56 | Compound 392 | S | A3 | A3 | B67 | B67 |
| Compound 393 | S | A3 | A3 | B68 | B68 | Compound 394 | S | A3 | A3 | B69 | B69 |
| Compound 395 | S | A3 | A3 | B70 | B70 | Compound 396 | S | A3 | A3 | B71 | B71 |
| Compound 397 | S | A3 | A3 | B72 | B72 | Compound 398 | S | A3 | A3 | B74 | B74 |
| Compound 399 | S | A3 | A3 | B79 | B79 | Compound 400 | S | A3 | A3 | B80 | B80 |
| Compound 401 | S | A3 | A3 | B82 | B82 | Compound 402 | S | A3 | A3 | B83 | B83 |
| Compound 403 | S | A3 | A3 | B86 | B86 | Compound 404 | S | A3 | A3 | B88 | B88 |
| Compound 405 | Se | A3 | A3 | B1 | B1 | Compound 406 | Se | A3 | A3 | B6 | B6 |
| Compound 407 | Se | A3 | A3 | B10 | B10 | Compound 408 | Se | A3 | A3 | B16 | B16 |
| Compound 409 | Se | A3 | A3 | B25 | B25 | Compound 410 | Se | A3 | A3 | B28 | B28 |
| Compound 411 | Se | A3 | A3 | B29 | B29 | Compound 412 | Se | A3 | A3 | B30 | B30 |
| Compound 413 | Se | A3 | A3 | B38 | B38 | Compound 414 | Se | A3 | A3 | B39 | B39 |
| Compound 415 | Se | A3 | A3 | B40 | B40 | Compound 416 | Se | A3 | A3 | B41 | B41 |
| Compound 417 | Se | A3 | A3 | B43 | B43 | Compound 418 | Se | A3 | A3 | B52 | B52 |
| Compound 419 | Se | A3 | A3 | B56 | B56 | Compound 420 | Se | A3 | A3 | B67 | B67 |
| Compound 421 | Se | A3 | A3 | B68 | B68 | Compound 422 | Se | A3 | A3 | B69 | B69 |
| Compound 423 | Se | A3 | A3 | B70 | B70 | Compound 424 | Se | A3 | A3 | B71 | B71 |
| Compound 425 | Se | A3 | A3 | B72 | B72 | Compound 426 | Se | A3 | A3 | B74 | B74 |
| Compound 427 | Se | A3 | A3 | B79 | B79 | Compound 428 | Se | A3 | A3 | B80 | B80 |
| Compound 429 | Se | A3 | A3 | B82 | B82 | Compound 430 | Se | A3 | A3 | B83 | B83 |
| Compound 431 | Se | A3 | A3 | B86 | B86 | Compound 432 | Se | A3 | A3 | B88 | B88 |
| Compound 433 | O | A4 | A4 | B1 | B1 | Compound 434 | O | A4 | A4 | B6 | B6 |
| Compound 435 | O | A4 | A4 | B10 | B10 | Compound 436 | O | A4 | A4 | B16 | B16 |
| Compound 437 | O | A4 | A4 | B25 | B25 | Compound 438 | O | A4 | A4 | B28 | B28 |
| Compound 439 | O | A4 | A4 | B29 | B29 | Compound 440 | O | A4 | A4 | B30 | B30 |
| Compound 441 | O | A4 | A4 | B38 | B38 | Compound 442 | O | A4 | A4 | B39 | B39 |
| Compound 443 | O | A4 | A4 | B40 | B40 | Compound 444 | O | A4 | A4 | B41 | B41 |
| Compound 445 | O | A4 | A4 | B43 | B43 | Compound 446 | O | A4 | A4 | B52 | B52 |
| Compound 447 | O | A4 | A4 | B56 | B56 | Compound 448 | O | A4 | A4 | B67 | B67 |
| Compound 449 | O | A4 | A4 | B68 | B68 | Compound 450 | O | A4 | A4 | B69 | B69 |
| Compound 451 | O | A4 | A4 | B70 | B70 | Compound 452 | O | A4 | A4 | B71 | B71 |
| Compound 453 | O | A4 | A4 | B72 | B72 | Compound 454 | O | A4 | A4 | B74 | B74 |
| Compound 455 | O | A4 | A4 | B79 | B79 | Compound 456 | O | A4 | A4 | B80 | B80 |
| Compound 457 | O | A4 | A4 | B82 | B82 | Compound 458 | O | A4 | A4 | B83 | B83 |
| Compound 459 | O | A4 | A4 | B86 | B86 | Compound 460 | O | A4 | A4 | B88 | B88 |
| Compound 461 | S | A4 | A4 | B1 | B1 | Compound 462 | S | A4 | A4 | B6 | B6 |
| Compound 463 | S | A4 | A4 | B10 | B10 | Compound 464 | S | A4 | A4 | B16 | B16 |
| Compound 465 | S | A4 | A4 | B25 | B25 | Compound 466 | S | A4 | A4 | B28 | B28 |
| Compound 467 | S | A4 | A4 | B29 | B29 | Compound 468 | S | A4 | A4 | B30 | B30 |
| Compound 469 | S | A4 | A4 | B38 | B38 | Compound 470 | S | A4 | A4 | B39 | B39 |
| Compound 471 | S | A4 | A4 | B40 | B40 | Compound 472 | S | A4 | A4 | B41 | B41 |
| Compound 473 | S | A4 | A4 | B43 | B43 | Compound 474 | S | A4 | A4 | B52 | B52 |
| Compound 475 | S | A4 | A4 | B56 | B56 | Compound 476 | S | A4 | A4 | B67 | B67 |
| Compound 477 | S | A4 | A4 | B68 | B68 | Compound 478 | S | A4 | A4 | B69 | B69 |
| Compound 479 | S | A4 | A4 | B70 | B70 | Compound 480 | S | A4 | A4 | B71 | B71 |
| Compound 481 | S | A4 | A4 | B72 | B72 | Compound 482 | S | A4 | A4 | B74 | B74 |
| Compound 483 | S | A4 | A4 | B79 | B79 | Compound 484 | S | A4 | A4 | B80 | B80 |
| Compound 485 | S | A4 | A4 | B82 | B82 | Compound 486 | S | A4 | A4 | B83 | B83 |
| Compound 487 | S | A4 | A4 | B86 | B86 | Compound 488 | S | A4 | A4 | B88 | B88 |
| Compound 489 | Se | A4 | A4 | B1 | B1 | Compound 490 | Se | A4 | A4 | B6 | B6 |
| Compound 491 | Se | A4 | A4 | B10 | B10 | Compound 492 | Se | A4 | A4 | B16 | B16 |
| Compound 493 | Se | A4 | A4 | B25 | B25 | Compound 494 | Se | A4 | A4 | B28 | B28 |
| Compound 495 | Se | A4 | A4 | B29 | B29 | Compound 496 | Se | A4 | A4 | B30 | B30 |
| Compound 497 | Se | A4 | A4 | B38 | B38 | Compound 498 | Se | A4 | A4 | B39 | B39 |
| Compound 499 | Se | A4 | A4 | B40 | B40 | Compound 500 | Se | A4 | A4 | B41 | B41 |
| Compound 501 | Se | A4 | A4 | B43 | B43 | Compound 502 | Se | A4 | A4 | B52 | B52 |
| Compound 503 | Se | A4 | A4 | B56 | B56 | Compound 504 | Se | A4 | A4 | B67 | B67 |
| Compound 505 | Se | A4 | A4 | B68 | B68 | Compound 506 | Se | A4 | A4 | B69 | B69 |
| Compound 507 | Se | A4 | A4 | B70 | B70 | Compound 508 | Se | A4 | A4 | B71 | B71 |
| Compound 509 | Se | A4 | A4 | B72 | B72 | Compound 510 | Se | A4 | A4 | B74 | B74 |
| Compound 511 | Se | A4 | A4 | B79 | B79 | Compound 512 | Se | A4 | A4 | B80 | B80 |
| Compound 513 | Se | A4 | A4 | B82 | B82 | Compound 514 | Se | A4 | A4 | B83 | B83 |
| Compound 515 | Se | A4 | A4 | B86 | B86 | Compound 516 | Se | A4 | A4 | B88 | B88 |
| Compound 517 | O | A5 | A5 | B1 | B1 | Compound 518 | O | A5 | A5 | B6 | B6 |
| Compound 519 | O | A5 | A5 | B10 | B10 | Compound 520 | O | A5 | A5 | B16 | B16 |
| Compound 521 | O | A5 | A5 | B25 | B25 | Compound 522 | O | A5 | A5 | B28 | B28 |
| Compound 523 | O | A5 | A5 | B29 | B29 | Compound 524 | O | A5 | A5 | B30 | B30 |
| Compound 525 | O | A5 | A5 | B38 | B38 | Compound 526 | O | A5 | A5 | B39 | B39 |
| Compound 527 | O | A5 | A5 | B40 | B40 | Compound 528 | O | A5 | A5 | B41 | B41 |
| Compound 529 | O | A5 | A5 | B43 | B43 | Compound 530 | O | A5 | A5 | B52 | B52 |
| Compound 531 | O | A5 | A5 | B56 | B56 | Compound 532 | O | A5 | A5 | B67 | B67 |
| Compound 533 | O | A5 | A5 | B68 | B68 | Compound 534 | O | A5 | A5 | B69 | B69 |
| Compound 535 | O | A5 | A5 | B70 | B70 | Compound 536 | O | A5 | A5 | B71 | B71 |
| Compound 537 | O | A5 | A5 | B72 | B72 | Compound 538 | O | A5 | A5 | B74 | B74 |
| Compound 539 | O | A5 | A5 | B79 | B79 | Compound 540 | O | A5 | A5 | B80 | B80 |
| Compound 541 | O | A5 | A5 | B82 | B82 | Compound 542 | O | A5 | A5 | B83 | B83 |
| Compound 543 | O | A5 | A5 | B86 | B86 | Compound 544 | O | A5 | A5 | B88 | B88 |
| Compound 545 | S | A5 | A5 | B1 | B1 | Compound 546 | S | A5 | A5 | B6 | B6 |
| Compound 547 | S | A5 | A5 | B10 | B10 | Compound 548 | S | A5 | A5 | B16 | B16 |
| Compound 549 | S | A5 | A5 | B25 | B25 | Compound 550 | S | A5 | A5 | B28 | B28 |
| Compound 551 | S | A5 | A5 | B29 | B29 | Compound 552 | S | A5 | A5 | B30 | B30 |
| Compound 553 | S | A5 | A5 | B38 | B38 | Compound 554 | S | A5 | A5 | B39 | B39 |
| Compound 555 | S | A5 | A5 | B40 | B40 | Compound 556 | S | A5 | A5 | B41 | B41 |
| Compound 557 | S | A5 | A5 | B43 | B43 | Compound 558 | S | A5 | A5 | B52 | B52 |
| Compound 559 | S | A5 | A5 | B56 | B56 | Compound 560 | S | A5 | A5 | B67 | B67 |
| Compound 561 | S | A5 | A5 | B68 | B68 | Compound 562 | S | A5 | A5 | B69 | B69 |
| Compound 563 | S | A5 | A5 | B70 | B70 | Compound 564 | S | A5 | A5 | B71 | B71 |
| Compound 565 | S | A5 | A5 | B72 | B72 | Compound 566 | S | A5 | A5 | B74 | B74 |
| Compound 567 | S | A5 | A5 | B79 | B79 | Compound 568 | S | A5 | A5 | B80 | B80 |
| Compound 569 | S | A5 | A5 | B82 | B82 | Compound 570 | S | A5 | A5 | B83 | B83 |
| Compound 571 | S | A5 | A5 | B86 | B86 | Compound 572 | S | A5 | A5 | B88 | B88 |
| Compound 573 | Se | A5 | A5 | B1 | B1 | Compound 574 | Se | A5 | A5 | B6 | B6 |
| Compound 575 | Se | A5 | A5 | B10 | B10 | Compound 576 | Se | A5 | A5 | B16 | B16 |
| Compound 577 | Se | A5 | A5 | B25 | B25 | Compound 578 | Se | A5 | A5 | B28 | B28 |
| Compound 579 | Se | A5 | A5 | B29 | B29 | Compound 580 | Se | A5 | A5 | B30 | B30 |
| Compound 581 | Se | A5 | A5 | B38 | B38 | Compound 582 | Se | A5 | A5 | B39 | B39 |
| Compound 583 | Se | A5 | A5 | B40 | B40 | Compound 584 | Se | A5 | A5 | B41 | B41 |
| Compound 585 | Se | A5 | A5 | B43 | B43 | Compound 586 | Se | A5 | A5 | B52 | B52 |
| Compound 587 | Se | A5 | A5 | B56 | B56 | Compound 588 | Se | A5 | A5 | B67 | B67 |
| Compound 589 | Se | A5 | A5 | B68 | B68 | Compound 590 | Se | A5 | A5 | B69 | B69 |
| Compound 591 | Se | A5 | A5 | B70 | B70 | Compound 592 | Se | A5 | A5 | B71 | B71 |
| Compound 593 | Se | A5 | A5 | B72 | B72 | Compound 594 | Se | A5 | A5 | B74 | B74 |
| Compound 595 | Se | A5 | A5 | B79 | B79 | Compound 596 | Se | A5 | A5 | B80 | B80 |
| Compound 597 | Se | A5 | A5 | B82 | B82 | Compound 598 | Se | A5 | A5 | B83 | B83 |
| Compound 599 | Se | A5 | A5 | B86 | B86 | Compound 600 | Se | A5 | A5 | B88 | B88 |
| Compound 601 | O | A6 | A6 | B1 | B1 | Compound 602 | O | A6 | A6 | B6 | B6 |
| Compound 603 | O | A6 | A6 | B10 | B10 | Compound 604 | O | A6 | A6 | B16 | B16 |
| Compound 605 | O | A6 | A6 | B25 | B25 | Compound 606 | O | A6 | A6 | B28 | B28 |
| Compound 607 | O | A6 | A6 | B29 | B29 | Compound 608 | O | A6 | A6 | B30 | B30 |
| Compound 609 | O | A6 | A6 | B38 | B38 | Compound 610 | O | A6 | A6 | B39 | B39 |
| Compound 611 | O | A6 | A6 | B40 | B40 | Compound 612 | O | A6 | A6 | B41 | B41 |
| Compound 613 | O | A6 | A6 | B43 | B43 | Compound 614 | O | A6 | A6 | B52 | B52 |
| Compound 615 | O | A6 | A6 | B56 | B56 | Compound 616 | O | A6 | A6 | B67 | B67 |
| Compound 617 | O | A6 | A6 | B68 | B68 | Compound 618 | O | A6 | A6 | B69 | B69 |
| Compound 619 | O | A6 | A6 | B70 | B70 | Compound 620 | O | A6 | A6 | B71 | B71 |
| Compound 621 | O | A6 | A6 | B72 | B72 | Compound 622 | O | A6 | A6 | B74 | B74 |
| Compound 623 | O | A6 | A6 | B79 | B79 | Compound 624 | O | A6 | A6 | B80 | B80 |
| Compound 625 | O | A6 | A6 | B82 | B82 | Compound 626 | O | A6 | A6 | B83 | B83 |
| Compound 627 | O | A6 | A6 | B86 | B86 | Compound 628 | O | A6 | A6 | B88 | B88 |
| Compound 629 | S | A6 | A6 | B1 | B1 | Compound 630 | S | A6 | A6 | B6 | B6 |
| Compound 631 | S | A6 | A6 | B10 | B10 | Compound 632 | S | A6 | A6 | B16 | B16 |
| Compound 633 | S | A6 | A6 | B25 | B25 | Compound 634 | S | A6 | A6 | B28 | B28 |
| Compound 635 | S | A6 | A6 | B29 | B29 | Compound 636 | S | A6 | A6 | B30 | B30 |
| Compound 637 | S | A6 | A6 | B38 | B38 | Compound 638 | S | A6 | A6 | B39 | B39 |
| Compound 639 | S | A6 | A6 | B40 | B40 | Compound 640 | S | A6 | A6 | B41 | B41 |
| Compound 641 | S | A6 | A6 | B43 | B43 | Compound 642 | S | A6 | A6 | B52 | B52 |
| Compound 643 | S | A6 | A6 | B56 | B56 | Compound 644 | S | A6 | A6 | B67 | B67 |
| Compound 645 | S | A6 | A6 | B68 | B68 | Compound 646 | S | A6 | A6 | B69 | B69 |
| Compound 647 | S | A6 | A6 | B70 | B70 | Compound 648 | S | A6 | A6 | B71 | B71 |
| Compound 649 | S | A6 | A6 | B72 | B72 | Compound 650 | S | A6 | A6 | B74 | B74 |
| Compound 651 | S | A6 | A6 | B79 | B79 | Compound 652 | S | A6 | A6 | B80 | B80 |
| Compound 653 | S | A6 | A6 | B82 | B82 | Compound 654 | S | A6 | A6 | B83 | B83 |
| Compound 655 | S | A6 | A6 | B86 | B86 | Compound 656 | S | A6 | A6 | B88 | B88 |
| Compound 657 | Se | A6 | A6 | B1 | B1 | Compound 658 | Se | A6 | A6 | B6 | B6 |
| Compound 659 | Se | A6 | A6 | B10 | B10 | Compound 660 | Se | A6 | A6 | B16 | B16 |
| Compound 661 | Se | A6 | A6 | B25 | B25 | Compound 662 | Se | A6 | A6 | B28 | B28 |
| Compound 663 | Se | A6 | A6 | B29 | B29 | Compound 664 | Se | A6 | A6 | B30 | B30 |
| Compound 665 | Se | A6 | A6 | B38 | B38 | Compound 666 | Se | A6 | A6 | B39 | B39 |
| Compound 667 | Se | A6 | A6 | B40 | B40 | Compound 668 | Se | A6 | A6 | B41 | B41 |
| Compound 669 | Se | A6 | A6 | B43 | B43 | Compound 670 | Se | A6 | A6 | B52 | B52 |
| Compound 671 | Se | A6 | A6 | B56 | B56 | Compound 672 | Se | A6 | A6 | B67 | B67 |
| Compound 673 | Se | A6 | A6 | B68 | B68 | Compound 674 | Se | A6 | A6 | B69 | B69 |
| Compound 675 | Se | A6 | A6 | B70 | B70 | Compound 676 | Se | A6 | A6 | B71 | B71 |
| Compound 677 | Se | A6 | A6 | B72 | B72 | Compound 678 | Se | A6 | A6 | B74 | B74 |
| Compound 679 | Se | A6 | A6 | B79 | B79 | Compound 680 | Se | A6 | A6 | B80 | B80 |
| Compound 681 | Se | A6 | A6 | B82 | B82 | Compound 682 | Se | A6 | A6 | B83 | B83 |
| Compound 683 | Se | A6 | A6 | B86 | B86 | Compound 684 | Se | A6 | A6 | B88 | B88 |
| Compound 685 | O | A7 | A7 | B1 | B1 | Compound 686 | O | A7 | A7 | B6 | B6 |
| Compound 687 | O | A7 | A7 | B10 | B10 | Compound 688 | O | A7 | A7 | B16 | B16 |
| Compound 689 | O | A7 | A7 | B25 | B25 | Compound 690 | O | A7 | A7 | B28 | B28 |
| Compound 691 | O | A7 | A7 | B29 | B29 | Compound 692 | O | A7 | A7 | B30 | B30 |
| Compound 693 | O | A7 | A7 | B38 | B38 | Compound 694 | O | A7 | A7 | B39 | B39 |
| Compound 695 | O | A7 | A7 | B40 | B40 | Compound 696 | O | A7 | A7 | B41 | B41 |
| Compound 697 | O | A7 | A7 | B43 | B43 | Compound 698 | O | A7 | A7 | B52 | B52 |
| Compound 699 | O | A7 | A7 | B56 | B56 | Compound 700 | O | A7 | A7 | B67 | B67 |
| Compound 701 | O | A7 | A7 | B68 | B68 | Compound 702 | O | A7 | A7 | B69 | B69 |
| Compound 703 | O | A7 | A7 | B70 | B70 | Compound 704 | O | A7 | A7 | B71 | B71 |
| Compound 705 | O | A7 | A7 | B72 | B72 | Compound 706 | O | A7 | A7 | B74 | B74 |
| Compound 707 | O | A7 | A7 | B79 | B79 | Compound 708 | O | A7 | A7 | B80 | B80 |
| Compound 709 | O | A7 | A7 | B82 | B82 | Compound 710 | O | A7 | A7 | B83 | B83 |
| Compound 711 | O | A7 | A7 | B86 | B86 | Compound 712 | O | A7 | A7 | B88 | B88 |
| Compound 713 | S | A7 | A7 | B1 | B1 | Compound 714 | S | A7 | A7 | B6 | B6 |
| Compound 715 | S | A7 | A7 | B10 | B10 | Compound 716 | S | A7 | A7 | B16 | B16 |
| Compound 717 | S | A7 | A7 | B25 | B25 | Compound 718 | S | A7 | A7 | B28 | B28 |
| Compound 719 | S | A7 | A7 | B29 | B29 | Compound 720 | S | A7 | A7 | B30 | B30 |
| Compound 721 | S | A7 | A7 | B38 | B38 | Compound 722 | S | A7 | A7 | B39 | B39 |
| Compound 723 | S | A7 | A7 | B40 | B40 | Compound 724 | S | A7 | A7 | B41 | B41 |
| Compound 725 | S | A7 | A7 | B43 | B43 | Compound 726 | S | A7 | A7 | B52 | B52 |
| Compound 727 | S | A7 | A7 | B56 | B56 | Compound 728 | S | A7 | A7 | B67 | B67 |
| Compound 729 | S | A7 | A7 | B68 | B68 | Compound 730 | S | A7 | A7 | B69 | B69 |
| Compound 731 | S | A7 | A7 | B70 | B70 | Compound 732 | S | A7 | A7 | B71 | B71 |
| Compound 733 | S | A7 | A7 | B72 | B72 | Compound 734 | S | A7 | A7 | B74 | B74 |
| Compound 735 | S | A7 | A7 | B79 | B79 | Compound 736 | S | A7 | A7 | B80 | B80 |
| Compound 737 | S | A7 | A7 | B82 | B82 | Compound 738 | S | A7 | A7 | B83 | B83 |
| Compound 739 | S | A7 | A7 | B86 | B86 | Compound 740 | S | A7 | A7 | B88 | B88 |
| Compound 741 | Se | A7 | A7 | B1 | B1 | Compound 742 | Se | A7 | A7 | B6 | B6 |
| Compound 743 | Se | A7 | A7 | B10 | B10 | Compound 744 | Se | A7 | A7 | B16 | B16 |
| Compound 745 | Se | A7 | A7 | B25 | B25 | Compound 746 | Se | A7 | A7 | B28 | B28 |
| Compound 747 | Se | A7 | A7 | B29 | B29 | Compound 748 | Se | A7 | A7 | B30 | B30 |
| Compound 749 | Se | A7 | A7 | B38 | B38 | Compound 750 | Se | A7 | A7 | B39 | B39 |
| Compound 751 | Se | A7 | A7 | B40 | B40 | Compound 752 | Se | A7 | A7 | B41 | B41 |
| Compound 753 | Se | A7 | A7 | B43 | B43 | Compound 754 | Se | A7 | A7 | B52 | B52 |
| Compound 755 | Se | A7 | A7 | B56 | B56 | Compound 756 | Se | A7 | A7 | B67 | B67 |
| Compound 757 | Se | A7 | A7 | B68 | B68 | Compound 758 | Se | A7 | A7 | B69 | B69 |
| Compound 759 | Se | A7 | A7 | B70 | B70 | Compound 760 | Se | A7 | A7 | B71 | B71 |
| Compound 761 | Se | A7 | A7 | B72 | B72 | Compound 762 | Se | A7 | A7 | B74 | B74 |
| Compound 763 | Se | A7 | A7 | B79 | B79 | Compound 764 | Se | A7 | A7 | B80 | B80 |
| Compound 765 | Se | A7 | A7 | B82 | B82 | Compound 766 | Se | A7 | A7 | B83 | B83 |
| Compound 767 | Se | A7 | A7 | B86 | B86 | Compound 768 | Se | A7 | A7 | B88 | B88 |
| Compound 769 | O | O | O | B1 | B1 | Compound 770 | O | O | O | B6 | B6 |
| Compound 771 | O | O | O | B10 | B10 | Compound 772 | O | O | O | B22 | B22 |
| Compound 773 | O | O | O | B25 | B25 | Compound 774 | O | O | O | B28 | B28 |
| Compound 775 | O | O | O | B29 | B29 | Compound 776 | O | O | O | B30 | B30 |
| Compound 777 | O | O | O | B38 | B38 | Compound 778 | O | O | O | B39 | B39 |
| Compound 779 | O | O | O | B40 | B40 | Compound 780 | O | O | O | B41 | B41 |
| Compound 781 | O | O | O | B43 | B43 | Compound 782 | O | O | O | B52 | B52 |
| Compound 783 | O | O | O | B56 | B56 | Compound 784 | O | O | O | B67 | B67 |
| Compound 785 | O | O | O | B68 | B68 | Compound 786 | O | O | O | B69 | B69 |
| Compound 787 | O | O | O | B70 | B70 | Compound 788 | O | O | O | B71 | B71 |
| Compound 789 | O | O | O | B72 | B72 | Compound 790 | O | O | O | B74 | B74 |
| Compound 791 | O | O | O | B79 | B79 | Compound 792 | O | O | O | B80 | B80 |
| Compound 793 | O | O | O | B82 | B82 | Compound 794 | O | O | O | B83 | B83 |
| Compound 795 | O | O | O | B86 | B86 | Compound 796 | O | O | O | B88 | B88 |
| Compound 797 | S | O | O | B1 | B1 | Compound 798 | S | O | O | B6 | B6 |
| Compound 799 | S | O | O | B10 | B10 | Compound 800 | S | O | O | B22 | B22 |
| Compound 801 | S | O | O | B25 | B25 | Compound 802 | S | O | O | B28 | B28 |
| Compound 803 | S | O | O | B29 | B29 | Compound 804 | S | O | O | B30 | B30 |
| Compound 805 | S | O | O | B38 | B38 | Compound 806 | S | O | O | B39 | B39 |
| Compound 807 | S | O | O | B40 | B40 | Compound 808 | S | O | O | B41 | B41 |
| Compound 809 | S | O | O | B43 | B43 | Compound 810 | S | O | O | B52 | B52 |
| Compound 811 | S | O | O | B56 | B56 | Compound 812 | S | O | O | B67 | B67 |
| Compound 813 | S | O | O | B68 | B68 | Compound 814 | S | O | O | B69 | B69 |
| Compound 815 | S | O | O | B70 | B70 | Compound 816 | S | O | O | B71 | B71 |
| Compound 817 | S | O | O | B72 | B72 | Compound 818 | S | O | O | B74 | B74 |
| Compound 819 | S | O | O | B79 | B79 | Compound 820 | S | O | O | B80 | B80 |
| Compound 821 | S | O | O | B82 | B82 | Compound 822 | S | O | O | B83 | B83 |
| Compound 823 | S | O | O | B86 | B86 | Compound 824 | S | O | O | B88 | B88 |
| Compound 825 | Se | O | O | B1 | B1 | Compound 826 | Se | O | O | B6 | B6 |
| Compound 827 | Se | O | O | B10 | B10 | Compound 828 | Se | O | O | B22 | B22 |
| Compound 829 | Se | O | O | B25 | B25 | Compound 830 | Se | O | O | B28 | B28 |
| Compound 831 | Se | O | O | B29 | B29 | Compound 832 | Se | O | O | B30 | B30 |
| Compound 833 | Se | O | O | B38 | B38 | Compound 834 | Se | O | O | B39 | B39 |
| Compound 835 | Se | O | O | B40 | B40 | Compound 836 | Se | O | O | B41 | B41 |
| Compound 837 | Se | O | O | B43 | B43 | Compound 838 | Se | O | O | B52 | B52 |
| Compound 839 | Se | O | O | B56 | B56 | Compound 840 | Se | O | O | B67 | B67 |
| Compound 841 | Se | O | O | B68 | B68 | Compound 842 | Se | O | O | B69 | B69 |
| Compound 843 | Se | O | O | B70 | B70 | Compound 844 | Se | O | O | B71 | B71 |
| Compound 845 | Se | O | O | B72 | B72 | Compound 846 | Se | O | O | B74 | B74 |
| Compound 847 | Se | O | O | B79 | B79 | Compound 848 | Se | O | O | B80 | B80 |
| Compound 849 | Se | O | O | B82 | B82 | Compound 850 | Se | O | O | B83 | B83 |
| Compound 851 | Se | O | O | B86 | B86 | Compound 852 | Se | O | O | B88 | B88 |
| Compound 853 | O | S | S | B1 | B1 | Compound 854 | O | O | O | B6 | B6 |
| Compound 855 | O | S | S | B10 | B10 | Compound 856 | O | S | S | B22 | B22 |
| Compound 857 | O | S | S | B25 | B25 | Compound 858 | O | S | S | B28 | B28 |
| Compound 859 | O | S | S | B29 | B29 | Compound 860 | O | S | S | B30 | B30 |
| Compound 861 | O | S | S | B38 | B38 | Compound 862 | O | S | S | B39 | B39 |
| Compound 863 | O | S | S | B40 | B40 | Compound 864 | O | S | S | B41 | B41 |
| Compound 865 | O | S | S | B43 | B43 | Compound 866 | O | S | S | B52 | B52 |
| Compound 867 | O | S | S | B56 | B56 | Compound 868 | O | S | S | B67 | B67 |
| Compound 869 | O | S | S | B68 | B68 | Compound 870 | O | S | S | B69 | B69 |
| Compound 871 | O | S | S | B70 | B70 | Compound 872 | O | S | S | B71 | B71 |
| Compound 873 | O | S | S | B72 | B72 | Compound 874 | O | S | S | B74 | B74 |
| Compound 875 | O | S | S | B79 | B79 | Compound 876 | O | S | S | B80 | B80 |
| Compound 877 | O | S | S | B82 | B82 | Compound 878 | O | S | S | B83 | B83 |
| Compound 879 | O | S | S | B86 | B86 | Compound 880 | O | S | S | B88 | B88 |
| Compound 881 | S | S | S | B1 | B1 | Compound 882 | S | S | S | B6 | B6 |
| Compound 883 | S | S | S | B10 | B10 | Compound 884 | S | S | S | B22 | B22 |
| Compound 885 | S | S | S | B25 | B25 | Compound 886 | S | S | S | B28 | B28 |
| Compound 887 | S | S | S | B29 | B29 | Compound 888 | S | S | S | B30 | B30 |
| Compound 889 | S | S | S | B38 | B38 | Compound 890 | S | S | S | B39 | B39 |
| Compound 891 | S | S | S | B40 | B40 | Compound 892 | S | S | S | B41 | B41 |
| Compound 893 | S | S | S | B43 | B43 | Compound 894 | S | S | S | B52 | B52 |
| Compound 895 | S | S | S | B56 | B56 | Compound 896 | S | S | S | B67 | B67 |
| Compound 897 | S | S | S | B68 | B68 | Compound 898 | S | S | S | B69 | B69 |
| Compound 899 | S | S | S | B70 | B70 | Compound 900 | S | S | S | B71 | B71 |
| Compound 901 | S | S | S | B72 | B72 | Compound 902 | S | S | S | B74 | B74 |
| Compound 903 | S | S | S | B79 | B79 | Compound 904 | S | S | S | B80 | B80 |
| Compound 905 | S | S | S | B82 | B82 | Compound 906 | S | S | S | B83 | B83 |
| Compound 907 | S | S | S | B86 | B86 | Compound 908 | S | S | S | B88 | B88 |
| Compound 909 | Se | S | S | B1 | B1 | Compound 910 | Se | S | S | B6 | B6 |
| Compound 911 | Se | S | S | B10 | B10 | Compound 912 | Se | S | S | B22 | B22 |
| Compound 913 | Se | S | S | B25 | B25 | Compound 914 | Se | S | S | B28 | B28 |
| Compound 915 | Se | S | S | B29 | B29 | Compound 916 | Se | S | S | B30 | B30 |
| Compound 917 | Se | S | S | B38 | B38 | Compound 918 | Se | S | S | B39 | B39 |
| Compound 919 | Se | S | S | B40 | B40 | Compound 920 | Se | S | S | B41 | B41 |
| Compound 921 | Se | S | S | B43 | B43 | Compound 922 | Se | S | S | B52 | B52 |
| Compound 923 | Se | S | S | B56 | B56 | Compound 924 | Se | S | S | B67 | B67 |
| Compound 925 | Se | S | S | B68 | B68 | Compound 926 | Se | S | S | B69 | B69 |
| Compound 927 | Se | S | S | B70 | B70 | Compound 928 | Se | S | S | B71 | B71 |
| Compound 929 | Se | S | S | B72 | B72 | Compound 930 | Se | S | S | B74 | B74 |
| Compound 931 | Se | S | S | B79 | B79 | Compound 932 | Se | S | S | B80 | B80 |
| Compound 933 | Se | S | S | B82 | B82 | Compound 934 | Se | S | S | B83 | B83 |
| Compound 935 | Se | S | S | B86 | B86 | Compound 936 | Se | S | S | B88 | B88 |
| Compound 937 | O | Se | Se | B1 | B1 | Compound 938 | O | Se | Se | B6 | B6 |
| Compound 939 | O | Se | Se | B10 | B10 | Compound 940 | O | Se | Se | B22 | B22 |
| Compound 941 | O | Se | Se | B25 | B25 | Compound 942 | O | Se | Se | B28 | B28 |
| Compound 943 | O | Se | Se | B29 | B29 | Compound 944 | O | Se | Se | B30 | B30 |
| Compound 945 | O | Se | Se | B38 | B38 | Compound 946 | O | Se | Se | B39 | B39 |
| Compound 947 | O | Se | Se | B40 | B40 | Compound 948 | O | Se | Se | B41 | B41 |
| Compound 949 | O | Se | Se | B43 | B43 | Compound 950 | O | Se | Se | B52 | B52 |
| Compound 951 | O | Se | Se | B56 | B56 | Compound 952 | O | Se | Se | B67 | B67 |
| Compound 953 | O | Se | Se | B68 | B68 | Compound 954 | O | Se | Se | B69 | B69 |
| Compound 955 | O | Se | Se | B70 | B70 | Compound 956 | O | Se | Se | B71 | B71 |
| Compound 957 | O | Se | Se | B72 | B72 | Compound 958 | O | Se | Se | B74 | B74 |
| Compound 959 | O | Se | Se | B79 | B79 | Compound 960 | O | Se | Se | B80 | B80 |
| Compound 961 | O | Se | Se | B82 | B82 | Compound 962 | O | Se | Se | B83 | B83 |
| Compound 963 | O | Se | Se | B86 | B86 | Compound 964 | O | Se | Se | B88 | B88 |
| Compound 965 | S | Se | Se | B1 | B1 | Compound 966 | S | Se | Se | B6 | B6 |
| Compound 967 | S | Se | Se | B10 | B10 | Compound 968 | S | Se | Se | B22 | B22 |
| Compound 969 | S | Se | Se | B25 | B25 | Compound 970 | S | Se | Se | B28 | B28 |
| Compound 971 | S | Se | Se | B29 | B29 | Compound 972 | S | Se | Se | B30 | B30 |
| Compound 973 | S | Se | Se | B38 | B38 | Compound 974 | S | Se | Se | B39 | B39 |
| Compound 975 | S | Se | Se | B40 | B40 | Compound 976 | S | Se | Se | B41 | B41 |
| Compound 977 | S | Se | Se | B43 | B43 | Compound 978 | S | Se | Se | B52 | B52 |
| Compound 979 | S | Se | Se | B56 | B56 | Compound 980 | S | Se | Se | B67 | B67 |
| Compound 981 | S | Se | Se | B68 | B68 | Compound 982 | S | Se | Se | B69 | B69 |
| Compound 983 | S | Se | Se | B70 | B70 | Compound 984 | S | Se | Se | B71 | B71 |
| Compound 985 | S | Se | Se | B72 | B72 | Compound 986 | S | Se | Se | B74 | B74 |
| Compound 987 | S | Se | Se | B79 | B79 | Compound 988 | S | Se | Se | B80 | B80 |
| Compound 989 | S | Se | Se | B82 | B82 | Compound 990 | S | Se | Se | B83 | B83 |
| Compound 991 | S | Se | Se | B86 | B86 | Compound 992 | S | Se | Se | B88 | B88 |
| Compound 993 | Se | Se | Se | B1 | B1 | Compound 994 | Se | Se | Se | B6 | B6 |
| Compound 995 | Se | Se | Se | B10 | B10 | Compound 996 | Se | Se | Se | B22 | B22 |
| Compound 997 | Se | Se | Se | B25 | B25 | Compound 998 | Se | Se | Se | B28 | B28 |
| Compound 999 | Se | Se | Se | B29 | B29 | Compound 1000 | Se | Se | Se | B30 | B30 |
| Compound 1001 | Se | Se | Se | B38 | B38 | Compound 1002 | Se | Se | Se | B39 | B39 |
| Compound 1003 | Se | Se | Se | B40 | B40 | Compound 1004 | Se | Se | Se | B41 | B41 |
| Compound 1005 | Se | Se | Se | B43 | B43 | Compound 1006 | Se | Se | Se | B52 | B52 |
| Compound 1007 | Se | Se | Se | B56 | B56 | Compound 1008 | Se | Se | Se | B67 | B67 |
| Compound 1009 | Se | Se | Se | B68 | B68 | Compound 1010 | Se | Se | Se | B69 | B69 |
| Compound 1011 | Se | Se | Se | B70 | B70 | Compound 1012 | Se | Se | Se | B71 | B71 |
| Compound 1013 | Se | Se | Se | B72 | B72 | Compound 1014 | Se | Se | Se | B74 | B74 |
| Compound 1015 | Se | Se | Se | B79 | B79 | Compound 1016 | Se | Se | Se | B80 | B80 |
| Compound 1017 | Se | Se | Se | B82 | B82 | Compound 1018 | Se | Se | Se | B83 | B83 |
| Compound 1019 | Se | Se | Se | B86 | B86 | Compound 1020 | Se | Se | Se | B88 | B88 |
| Compound 1021 | O | A1 | A1 | B1 | B6 | Compound 1022 | O | A1 | A1 | B2 | B6 |
| Compound 1023 | O | A1 | A1 | B25 | B26 | Compound 1024 | O | A1 | A1 | B27 | B28 |
| Compound 1025 | O | A1 | A1 | B29 | B30 | Compound 1026 | O | A1 | A1 | B39 | B40 |
| Compound 1027 | O | A1 | A1 | B54 | B41 | Compound 1028 | O | A1 | A1 | B54 | B52 |
| Compound 1029 | O | A1 | A1 | B52 | B56 | Compound 1030 | O | A1 | A1 | B55 | B56 |
| Compound 1031 | O | A1 | A1 | B64 | B56 | Compound 1032 | O | A1 | A1 | B68 | B69 |
| Compound 1033 | O | A1 | A1 | B69 | B70 | Compound 1034 | O | A1 | A1 | B71 | B72 |
| Compound 1035 | O | A1 | A1 | B68 | B80 | Compound 1036 | O | A1 | A1 | B68 | B83 |
| Compound 1037 | S | A1 | A1 | B1 | B6 | Compound 1038 | S | A1 | A1 | B2 | B6 |
| Compound 1039 | S | A1 | A1 | B25 | B26 | Compound 1040 | S | A1 | A1 | B27 | B28 |
| Compound 1041 | S | A1 | A1 | B29 | B30 | Compound 1042 | S | A1 | A1 | B39 | B40 |
| Compound 1043 | S | A1 | A1 | B54 | B41 | Compound 1044 | S | A1 | A1 | B54 | B52 |
| Compound 1045 | S | A1 | A1 | B52 | B56 | Compound 1046 | S | A1 | A1 | B55 | B56 |
| Compound 1047 | S | A1 | A1 | B64 | B56 | Compound 1048 | S | A1 | A1 | B68 | B69 |
| Compound 1049 | S | A1 | A1 | B69 | B70 | Compound 1050 | S | A1 | A1 | B71 | B72 |
| Compound 1051 | S | A1 | A1 | B68 | B80 | Compound 1052 | S | A1 | A1 | B68 | B83 |
| Compound 1053 | Se | A1 | A1 | B1 | B6 | Compound 1054 | Se | A1 | A1 | B2 | B6 |
| Compound 1055 | Se | A1 | A1 | B25 | B26 | Compound 1056 | Se | A1 | A1 | B27 | B28 |
| Compound 1057 | Se | A1 | A1 | B29 | B30 | Compound 1058 | Se | A1 | A1 | B39 | B40 |
| Compound 1059 | Se | A1 | A1 | B54 | B41 | Compound 1060 | Se | A1 | A1 | B54 | B52 |
| Compound 1061 | Se | A1 | A1 | B52 | B56 | Compound 1062 | Se | A1 | A1 | B55 | B56 |
| Compound 1063 | Se | A1 | A1 | B64 | B56 | Compound 1064 | Se | A1 | A1 | B68 | B69 |
| Compound 1065 | Se | A1 | A1 | B69 | B70 | Compound 1066 | Se | A1 | A1 | B71 | B72 |
| Compound 1067 | Se | A1 | A1 | B68 | B80 | Compound 1068 | Se | A1 | A1 | B68 | B83 |
| Compound 1069 | O | A2 | A2 | B1 | B6 | Compound 1070 | O | A2 | A2 | B2 | B6 |
| Compound 1071 | O | A2 | A2 | B25 | B26 | Compound 1072 | O | A2 | A2 | B27 | B28 |
| Compound 1073 | O | A2 | A2 | B29 | B30 | Compound 1074 | O | A2 | A2 | B39 | B40 |
| Compound 1075 | O | A2 | A2 | B54 | B41 | Compound 1076 | O | A2 | A2 | B54 | B52 |
| Compound 1077 | O | A2 | A2 | B52 | B56 | Compound 1078 | O | A2 | A2 | B55 | B56 |
| Compound 1079 | O | A2 | A2 | B64 | B56 | Compound 1080 | O | A2 | A2 | B68 | B69 |
| Compound 1081 | O | A2 | A2 | B69 | B70 | Compound 1082 | O | A2 | A2 | B71 | B72 |
| Compound 1083 | O | A2 | A2 | B68 | B80 | Compound 1084 | O | A2 | A2 | B68 | B83 |
| Compound 1085 | S | A2 | A2 | B1 | B6 | Compound 1086 | S | A2 | A2 | B2 | B6 |
| Compound 1087 | S | A2 | A2 | B25 | B26 | Compound 1088 | S | A2 | A2 | B27 | B28 |
| Compound 1089 | S | A2 | A2 | B29 | B30 | Compound 1090 | S | A2 | A2 | B39 | B40 |
| Compound 1091 | S | A2 | A2 | B54 | B41 | Compound 1092 | S | A2 | A2 | B54 | B52 |
| Compound 1093 | S | A2 | A2 | B52 | B56 | Compound 1094 | S | A2 | A2 | B55 | B56 |
| Compound 1095 | S | A2 | A2 | B64 | B56 | Compound 1096 | S | A2 | A2 | B68 | B69 |
| Compound 1097 | S | A2 | A2 | B69 | B70 | Compound 1098 | S | A2 | A2 | B71 | B72 |
| Compound 1099 | S | A2 | A2 | B68 | B80 | Compound 1100 | S | A2 | A2 | B68 | B83 |
| Compound 1101 | Se | A2 | A2 | B1 | B6 | Compound 1102 | Se | A2 | A2 | B2 | B6 |
| Compound 1103 | Se | A2 | A2 | B25 | B26 | Compound 1104 | Se | A2 | A2 | B27 | B28 |
| Compound 1105 | Se | A2 | A2 | B29 | B30 | Compound 1106 | Se | A2 | A2 | B39 | B40 |
| Compound 1107 | Se | A2 | A2 | B54 | B41 | Compound 1108 | Se | A2 | A2 | B54 | B52 |
| Compound 1109 | Se | A2 | A2 | B52 | B56 | Compound 1110 | Se | A2 | A2 | B55 | B56 |
| Compound 1111 | Se | A2 | A2 | B64 | B56 | Compound 1112 | Se | A2 | A2 | B68 | B69 |
| Compound 1113 | Se | A2 | A2 | B69 | B70 | Compound 1114 | Se | A2 | A2 | B71 | B72 |
| Compound 1115 | Se | A2 | A2 | B68 | B80 | Compound 1116 | Se | A2 | A2 | B68 | B83 |
| Compound 1117 | O | A3 | A3 | B1 | B1 | Compound 1118 | O | A3 | A3 | B6 | B6 |
| Compound 1119 | O | A3 | A3 | B25 | B25 | Compound 1120 | O | A3 | A3 | B28 | B28 |
| Compound 1121 | O | A3 | A3 | B29 | B29 | Compound 1122 | O | A3 | A3 | B30 | B30 |
| Compound 1123 | O | A3 | A3 | B56 | B56 | Compound 1124 | O | A3 | A3 | B67 | B67 |
| Compound 1125 | O | A3 | A3 | B68 | B68 | Compound 1126 | O | A3 | A3 | B69 | B69 |
| Compound 1127 | O | A3 | A3 | B70 | B70 | Compound 1128 | O | A3 | A3 | B71 | B71 |
| Compound 1129 | O | A3 | A3 | B72 | B72 | Compound 1130 | O | A3 | A3 | B74 | B74 |
| Compound 1131 | O | A3 | A3 | B80 | B80 | Compound 1132 | O | A3 | A3 | B83 | B83 |
| Compound 1133 | S | A3 | A3 | B1 | B1 | Compound 1134 | S | A3 | A3 | B6 | B6 |
| Compound 1135 | S | A3 | A3 | B25 | B25 | Compound 1136 | S | A3 | A3 | B28 | B28 |
| Compound 1137 | S | A3 | A3 | B29 | B29 | Compound 1138 | S | A3 | A3 | B30 | B30 |
| Compound 1139 | S | A3 | A3 | B56 | B56 | Compound 1140 | S | A3 | A3 | B67 | B67 |
| Compound 1141 | S | A3 | A3 | B68 | B68 | Compound 1142 | S | A3 | A3 | B69 | B69 |
| Compound 1143 | S | A3 | A3 | B70 | B70 | Compound 1144 | S | A3 | A3 | B71 | B71 |
| Compound 1145 | S | A3 | A3 | B72 | B72 | Compound 1146 | S | A3 | A3 | B74 | B74 |
| Compound 1147 | S | A3 | A3 | B80 | B80 | Compound 1148 | S | A3 | A3 | B83 | B83 |
| Compound 1149 | Se | A3 | A3 | B1 | B1 | Compound 1150 | Se | A3 | A3 | B6 | B6 |
| Compound 1151 | Se | A3 | A3 | B25 | B25 | Compound 1152 | Se | A3 | A3 | B28 | B28 |
| Compound 1153 | Se | A3 | A3 | B29 | B29 | Compound 1154 | Se | A3 | A3 | B30 | B30 |
| Compound 1155 | Se | A3 | A3 | B56 | B56 | Compound 1156 | Se | A3 | A3 | B67 | B67 |
| Compound 1157 | Se | A3 | A3 | B68 | B68 | Compound 1158 | Se | A3 | A3 | B69 | B69 |
| Compound 1159 | Se | A3 | A3 | B70 | B70 | Compound 1160 | Se | A3 | A3 | B71 | B71 |
| Compound 1161 | Se | A3 | A3 | B72 | B72 | Compound 1162 | Se | A3 | A3 | B74 | B74 |
| Compound 1163 | Se | A3 | A3 | B80 | B80 | Compound 1164 | Se | A3 | A3 | B83 | B83 |
| Compound 1165 | O | O | A1 | B1 | B1 | Compound 1166 | O | O | A1 | B6 | B6 |
| Compound 1167 | O | O | A1 | B25 | B25 | Compound 1168 | O | O | A1 | B28 | B28 |
| Compound 1169 | O | O | A1 | B29 | B29 | Compound 1170 | O | O | A1 | B30 | B30 |
| Compound 1171 | O | O | A1 | B56 | B56 | Compound 1172 | O | O | A1 | B67 | B67 |
| Compound 1173 | O | O | A1 | B68 | B68 | Compound 1174 | O | O | A1 | B69 | B69 |
| Compound 1175 | O | O | A1 | B70 | B70 | Compound 1176 | O | O | A1 | B71 | B71 |
| Compound 1177 | O | O | A1 | B72 | B72 | Compound 1178 | O | O | A1 | B74 | B74 |
| Compound 1179 | O | O | A1 | B80 | B80 | Compound 1180 | O | O | A1 | B83 | B83 |
| Compound 1181 | S | O | A1 | B1 | B1 | Compound 1182 | S | O | A1 | B6 | B6 |
| Compound 1183 | S | O | A1 | B25 | B25 | Compound 1184 | S | O | A1 | B28 | B28 |
| Compound 1185 | S | O | A1 | B29 | B29 | Compound 1186 | S | O | A1 | B30 | B30 |
| Compound 1187 | S | O | A1 | B56 | B56 | Compound 1188 | S | O | A1 | B67 | B67 |
| Compound 1189 | S | O | A1 | B68 | B68 | Compound 1190 | S | O | A1 | B69 | B69 |
| Compound 1191 | S | O | A1 | B70 | B70 | Compound 1192 | S | O | A1 | B71 | B71 |
| Compound 1193 | S | O | A1 | B72 | B72 | Compound 1194 | S | O | A1 | B74 | B74 |
| Compound 1195 | S | O | A1 | B80 | B80 | Compound 1196 | S | O | A1 | B83 | B83 |
| Compound 1197 | Se | O | A1 | B1 | B1 | Compound 1198 | Se | O | A1 | B6 | B6 |
| Compound 1199 | Se | O | A1 | B25 | B25 | Compound 1200 | Se | O | A1 | B28 | B28 |
| Compound 1201 | Se | O | A1 | B29 | B29 | Compound 1202 | Se | O | A1 | B30 | B30 |
| Compound 1203 | Se | O | A1 | B56 | B56 | Compound 1204 | Se | O | A1 | B67 | B67 |
| Compound 1205 | Se | O | A1 | B68 | B68 | Compound 1206 | Se | O | A1 | B69 | B69 |
| Compound 1207 | Se | O | A1 | B70 | B70 | Compound 1208 | Se | O | A1 | B71 | B71 |
| Compound 1209 | Se | O | A1 | B72 | B72 | Compound 1210 | Se | O | A1 | B74 | B74 |
| Compound 1211 | Se | O | A1 | B80 | B80 | Compound 1212 | Se | O | A1 | B83 | B83 |
| Compound 1213 | O | A1 | A2 | B1 | B1 | Compound 1214 | O | A1 | A2 | B6 | B6 |
| Compound 1215 | O | A1 | A2 | B25 | B25 | Compound 1216 | O | A1 | A2 | B28 | B28 |
| Compound 1217 | O | A1 | A2 | B29 | B29 | Compound 1218 | O | A1 | A2 | B30 | B30 |
| Compound 1219 | O | A1 | A2 | B56 | B56 | Compound 1220 | O | A1 | A2 | B67 | B67 |
| Compound 1221 | O | A1 | A2 | B68 | B68 | Compound 1222 | O | A1 | A2 | B69 | B69 |
| Compound 1223 | O | A1 | A2 | B70 | B70 | Compound 1224 | O | A1 | A2 | B71 | B71 |
| Compound 1225 | O | A1 | A2 | B72 | B72 | Compound 1226 | O | A1 | A2 | B74 | B74 |
| Compound 1227 | O | A1 | A2 | B80 | B80 | Compound 1228 | O | A1 | A2 | B83 | B83 |
| Compound 1229 | S | A1 | A2 | B1 | B1 | Compound 1230 | S | A1 | A2 | B6 | B6 |
| Compound 1231 | S | A1 | A2 | B25 | B25 | Compound 1232 | S | A1 | A2 | B28 | B28 |
| Compound 1233 | S | A1 | A2 | B29 | B29 | Compound 1234 | S | A1 | A2 | B30 | B30 |
| Compound 1235 | S | A1 | A2 | B56 | B56 | Compound 1236 | S | A1 | A2 | B67 | B67 |
| Compound 1237 | S | A1 | A2 | B68 | B68 | Compound 1238 | S | A1 | A2 | B69 | B69 |
| Compound 1239 | S | A1 | A2 | B70 | B70 | Compound 1240 | S | A1 | A2 | B71 | B71 |
| Compound 1241 | S | A1 | A2 | B72 | B72 | Compound 1242 | S | A1 | A2 | B74 | B74 |
| Compound 1243 | S | A1 | A2 | B80 | B80 | Compound 1244 | S | A1 | A2 | B83 | B83 |
| Compound 1245 | Se | A1 | A2 | B1 | B1 | Compound 1246 | Se | A1 | A2 | B6 | B6 |
| Compound 1247 | Se | A1 | A2 | B25 | B25 | Compound 1248 | Se | A1 | A2 | B28 | B28 |
| Compound 1249 | Se | A1 | A2 | B29 | B29 | Compound 1250 | Se | A1 | A2 | B30 | B30 |
| Compound 1251 | Se | A1 | A2 | B56 | B56 | Compound 1252 | Se | A1 | A2 | B67 | B67 |
| Compound 1253 | Se | A1 | A2 | B68 | B68 | Compound 1254 | Se | A1 | A2 | B69 | B69 |
| Compound 1255 | Se | A1 | A2 | B70 | B70 | Compound 1256 | Se | A1 | A2 | B71 | B71 |
| Compound 1257 | Se | A1 | A2 | B72 | B72 | Compound 1258 | Se | A1 | A2 | B74 | B74 |
| Compound 1259 | Se | A1 | A2 | B80 | B80 | Compound 1260 | Se | A1 | A2 | B83 | B83 |
| Compound 1261 | O | A1 | A3 | B1 | B1 | Compound 1262 | O | A1 | A3 | B6 | B6 |
| Compound 1263 | O | A1 | A3 | B25 | B25 | Compound 1264 | O | A1 | A3 | B28 | B28 |
| Compound 1265 | O | A1 | A3 | B29 | B29 | Compound 1266 | O | A1 | A3 | B30 | B30 |
| Compound 1267 | O | A1 | A3 | B56 | B56 | Compound 1268 | O | A1 | A3 | B67 | B67 |
| Compound 1269 | O | A1 | A3 | B68 | B68 | Compound 1270 | O | A1 | A3 | B69 | B69 |
| Compound 1271 | O | A1 | A3 | B70 | B70 | Compound 1272 | O | A1 | A3 | B71 | B71 |
| Compound 1273 | O | A1 | A3 | B72 | B72 | Compound 1274 | O | A1 | A3 | B74 | B74 |
| Compound 1275 | O | A1 | A3 | B80 | B80 | Compound 1276 | O | A1 | A3 | B83 | B83 |
| Compound 1277 | S | A1 | A3 | B1 | B1 | Compound 1278 | S | A1 | A3 | B6 | B6 |
| Compound 1279 | S | A1 | A3 | B25 | B25 | Compound 1280 | S | A1 | A3 | B28 | B28 |
| Compound 1281 | S | A1 | A3 | B29 | B29 | Compound 1282 | S | A1 | A3 | B30 | B30 |
| Compound 1283 | S | A1 | A3 | B56 | B56 | Compound 1284 | S | A1 | A3 | B67 | B67 |
| Compound 1285 | S | A1 | A3 | B68 | B68 | Compound 1286 | S | A1 | A3 | B69 | B69 |
| Compound 1287 | S | A1 | A3 | B70 | B70 | Compound 1288 | S | A1 | A3 | B71 | B71 |
| Compound 1289 | S | A1 | A3 | B72 | B72 | Compound 1290 | S | A1 | A3 | B74 | B74 |
| Compound 1291 | S | A1 | A3 | B80 | B80 | Compound 1292 | S | A1 | A3 | B83 | B83 |
| Compound 1293 | Se | A1 | A3 | B1 | B1 | Compound 1294 | Se | A1 | A3 | B6 | B6 |
| Compound 1295 | Se | A1 | A3 | B25 | B25 | Compound 1296 | Se | A1 | A3 | B28 | B28 |
| Compound 1297 | Se | A1 | A3 | B29 | B29 | Compound 1298 | Se | A1 | A3 | B30 | B30 |
| Compound 1299 | Se | A1 | A3 | B56 | B56 | Compound 1300 | Se | A1 | A3 | B67 | B67 |
| Compound 1301 | Se | A1 | A3 | B68 | B68 | Compound 1302 | Se | A1 | A3 | B69 | B69 |
| Compound 1303 | Se | A1 | A3 | B70 | B70 | Compound 1304 | Se | A1 | A3 | B71 | B71 |
| Compound 1305 | Se | A1 | A3 | B72 | B72 | Compound 1306 | Se | A1 | A3 | B74 | B74 |
| Compound 1307 | Se | A1 | A3 | B80 | B80 | Compound 1308 | Se | A1 | A3 | B83 | B83 |
| Compound 1309 | O | A2 | A6 | B1 | B1 | Compound 1310 | O | A2 | A6 | B6 | B6 |
| Compound 1311 | O | A2 | A6 | B25 | B25 | Compound 1312 | O | A2 | A6 | B28 | B28 |
| Compound 1313 | O | A2 | A6 | B29 | B29 | Compound 1314 | O | A2 | A6 | B30 | B30 |
| Compound 1315 | O | A2 | A6 | B56 | B56 | Compound 1316 | O | A2 | A6 | B67 | B67 |
| Compound 1317 | O | A2 | A6 | B68 | B68 | Compound 1318 | O | A2 | A6 | B69 | B69 |
| Compound 1319 | O | A2 | A6 | B70 | B70 | Compound 1320 | O | A2 | A6 | B71 | B71 |
| Compound 1321 | O | A2 | A6 | B72 | B72 | Compound 1322 | O | A2 | A6 | B74 | B74 |
| Compound 1323 | O | A2 | A6 | B80 | B80 | Compound 1324 | O | A2 | A6 | B83 | B83 |
| Compound 1325 | S | A2 | A6 | B1 | B1 | Compound 1326 | S | A2 | A6 | B6 | B6 |
| Compound 1327 | S | A2 | A6 | B25 | B25 | Compound 1328 | S | A2 | A6 | B28 | B28 |
| Compound 1329 | S | A2 | A6 | B29 | B29 | Compound 1330 | S | A2 | A6 | B30 | B30 |
| Compound 1331 | S | A2 | A6 | B56 | B56 | Compound 1332 | S | A2 | A6 | B67 | B67 |
| Compound 1333 | S | A2 | A6 | B68 | B68 | Compound 1334 | S | A2 | A6 | B69 | B69 |
| Compound 1335 | S | A2 | A6 | B70 | B70 | Compound 1336 | S | A2 | A6 | B71 | B71 |
| Compound 1337 | S | A2 | A6 | B72 | B72 | Compound 1338 | S | A2 | A6 | B74 | B74 |
| Compound 1339 | S | A2 | A6 | B80 | B80 | Compound 1340 | S | A2 | A6 | B83 | B83 |
| Compound 1341 | Se | A2 | A6 | B1 | B1 | Compound 1342 | Se | A2 | A6 | B6 | B6 |
| Compound 1343 | Se | A2 | A6 | B25 | B25 | Compound 1344 | Se | A2 | A6 | B28 | B28 |
| Compound 1345 | Se | A2 | A6 | B29 | B29 | Compound 1346 | Se | A2 | A6 | B30 | B30 |
| Compound 1347 | Se | A2 | A6 | B56 | B56 | Compound 1348 | Se | A2 | A6 | B67 | B67 |
| Compound 1349 | Se | A2 | A6 | B68 | B68 | Compound 1350 | Se | A2 | A6 | B69 | B69 |
| Compound 1351 | Se | A2 | A6 | B70 | B70 | Compound 1352 | Se | A2 | A6 | B71 | B71 |
| Compound 1353 | Se | A2 | A6 | B72 | B72 | Compound 1354 | Se | A2 | A6 | B74 | B74 |
| Compound 1355 | Se | A2 | A6 | B80 | B80 | Compound 1356 | Se | A2 | A6 | B83 | B83 |

According to an embodiment of the present disclosure, in Formula 2, L is selected from substituted or unsubstituted phenylene, substituted or unsubstituted biphenylene, substituted or unsubstituted terphenylene, substituted or unsubstituted naphthylene, substituted or unsubstituted fluorenylidene, substituted or unsubstituted silafluorenylidene, substituted or unsubstituted carbazolylene, substituted or unsubstituted dibenzofurylene, substituted or unsubstituted dibenzothienylene, substituted or unsubstituted dibenzoselenophenylene, substituted or unsubstituted phenanthrylene, substituted or unsubstituted triphenylenylene, substituted or unsubstituted pyridylene, substituted or unsubstituted spirobifluorenylidene, substituted or unsubstituted anthrylene, substituted or unsubstituted pyrenylene or a combination thereof; preferably, L is selected from substituted or unsubstituted phenylene or substituted or unsubstituted biphenylene; more preferably, L is phenylene or biphenylene.

According to an embodiment of the present disclosure, in Formula 2, R₁ is selected from hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms or substituted or unsubstituted heteroaryl having 3 to 30 carbon atoms; preferably, R₁ is selected from hydrogen, deuterium, substituted or unsubstituted aryl having 6 to 30 carbon atoms or substituted or unsubstituted heteroaryl having 3 to 30 carbon atoms.

According to an embodiment of the present disclosure, in Formula 2, Ar₁ and Ar₂ are selected from substituted or unsubstituted aryl having 6 to 20 carbon atoms or substituted or unsubstituted heteroaryl having 3 to 20 carbon atoms; preferably, Ar₁ and Ar₂ are selected from phenyl, biphenyl, terphenyl, naphthyl, fluorenyl, dibenzothienyl, spirobifluorenyl, pyridyl or pyrimidinyl.

According to an embodiment of the present disclosure, the compound having the structure of Formula 2 is selected from the group consisting of the following compounds:

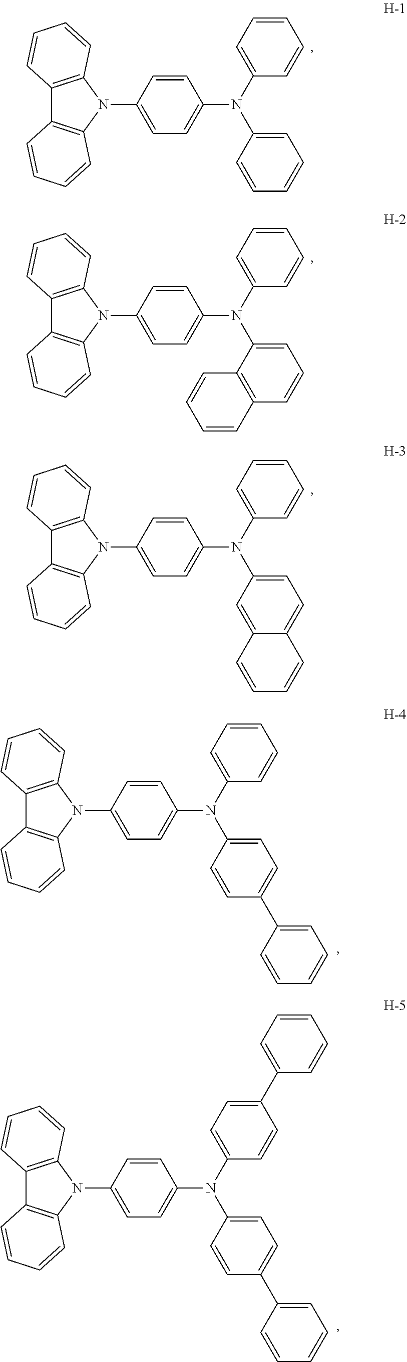

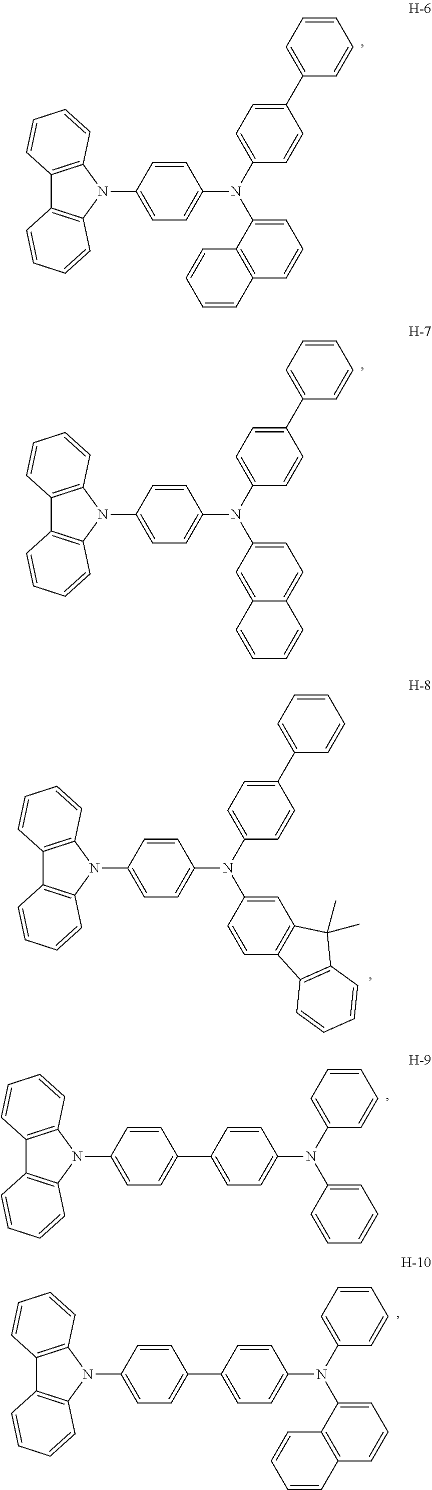

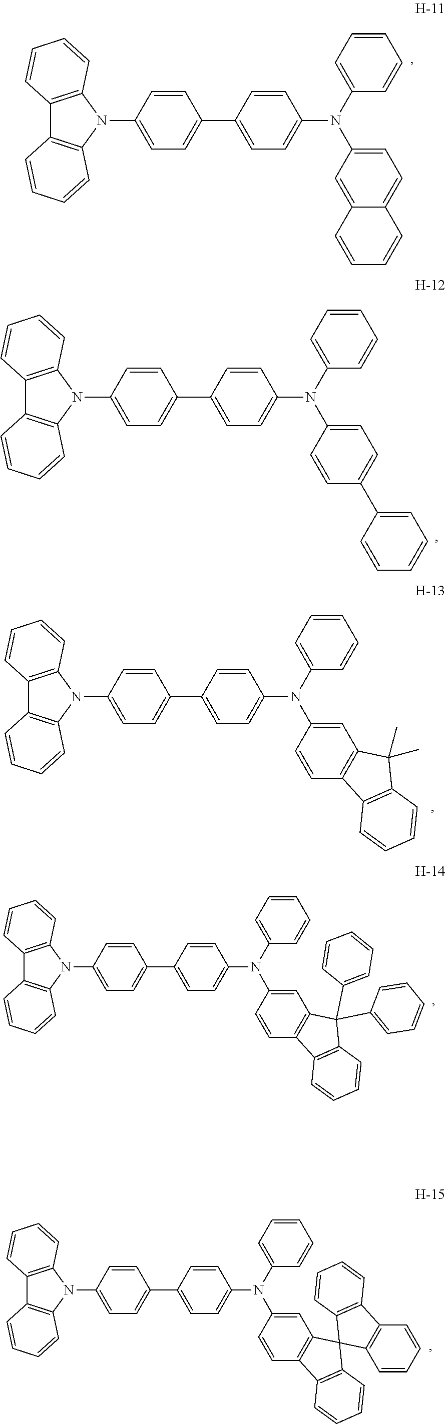 [SMILES: c1ccc(N(c2ccc(-c3ccc(-n4c5ccccc5c5ccccc54)cc3)cc2)c2ccc3ccccc3c2)cc1 ; c1ccc(-c2ccc(N(c3ccccc3)c3ccc(-c4ccc(-n5c6ccccc6c6ccccc65)cc4)cc3)cc2)cc1 ; CC1(C)c2ccccc2-c2ccc(N(c3ccccc3)c3ccc(-c4ccc(-n5c6ccccc6c6ccccc65)cc4)cc3)cc21 ; c1ccc(N(c2ccc(-c3ccc(-n4c5ccccc5c5ccccc54)cc3)cc2)c2ccc3c(c2)C(c2ccccc2)(c2ccccc2)c2ccccc2-3)cc1 ; c1ccc(N(c2ccc(-c3ccc(-n4c5ccccc5c5ccccc54)cc3)cc2)c2ccc3c(c2)C2(c4ccccc4-c4ccccc42)c2ccccc2-3)cc1]

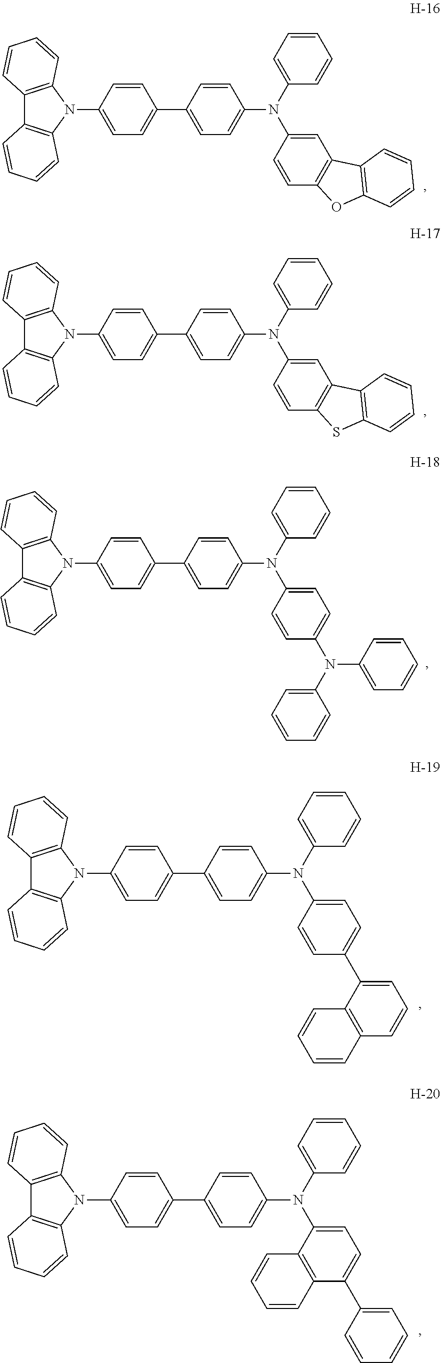 [SMILES: c1ccc(N(c2ccc(-c3ccc(-n4c5ccccc5c5ccccc54)cc3)cc2)c2ccc3oc4ccccc4c3c2)cc1 ; c1ccc(N(c2ccc(-c3ccc(-n4c5ccccc5c5ccccc54)cc3)cc2)c2ccc3sc4ccccc4c3c2)cc1 ; c1ccc(N(c2ccccc2)c2ccc(N(c3ccccc3)c3ccc(-c4ccc(-n5c6ccccc6c6ccccc65)cc4)cc3)cc2)cc1 ; c1ccc(N(c2ccc(-c3ccc(-n4c5ccccc5c5ccccc54)cc3)cc2)c2ccc(-c3cccc4ccccc34)cc2)cc1 ; c1ccc(-c2ccc(N(c3ccccc3)c3ccc(-c4ccc(-n5c6ccccc6c6ccccc65)cc4)cc3)c3ccccc23)cc1]

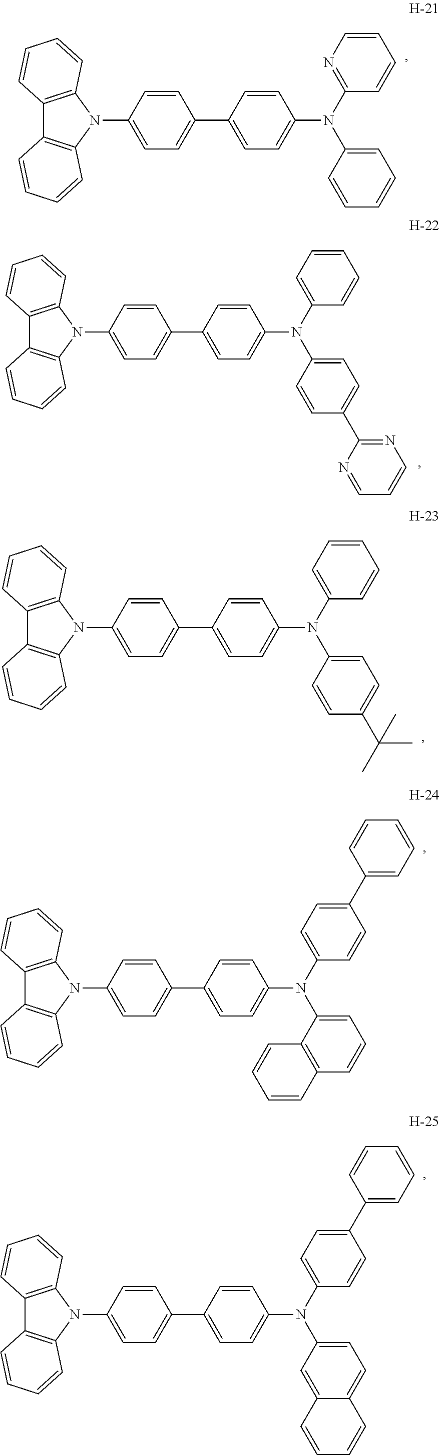

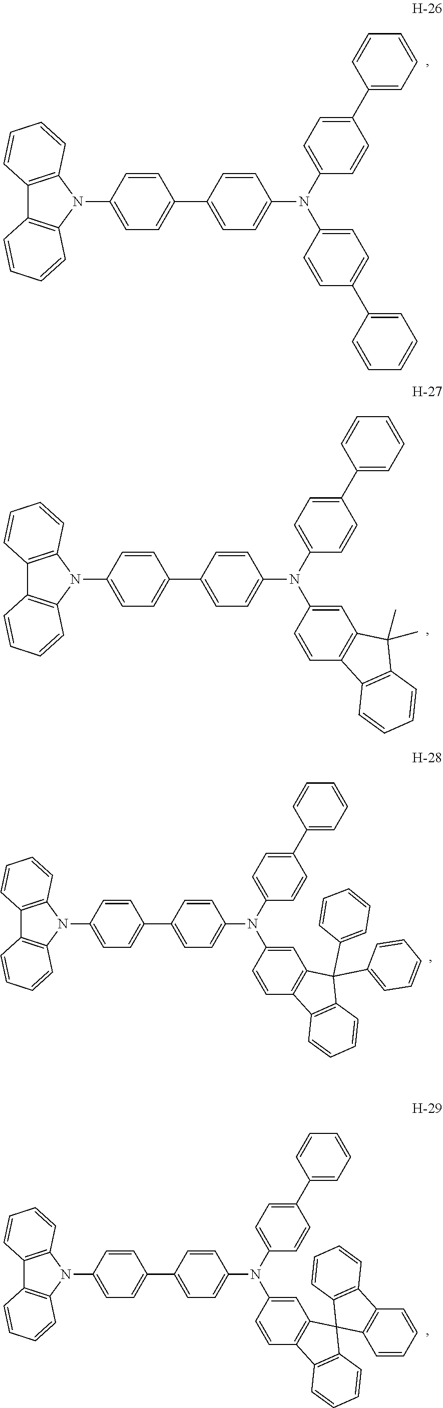 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc(-c4ccc(-n5c6ccccc6c6ccccc65)cc4)cc3)cc21 ; c1ccc(-c2ccc(N(c3ccc(-c4ccc(-n5c6ccccc6c6ccccc65)cc4)cc3)c3ccc4c(c3)C(c3ccccc3)(c3ccccc3)c3ccccc3-4)cc2)cc1 ; c1ccc(-c2ccc(N(c3ccc(-c4ccc(-n5c6ccccc6c6ccccc65)cc4)cc3)c3ccc4c(c3)C3(c5ccccc5-c5ccccc53)c3ccccc3-4)cc2)cc1]

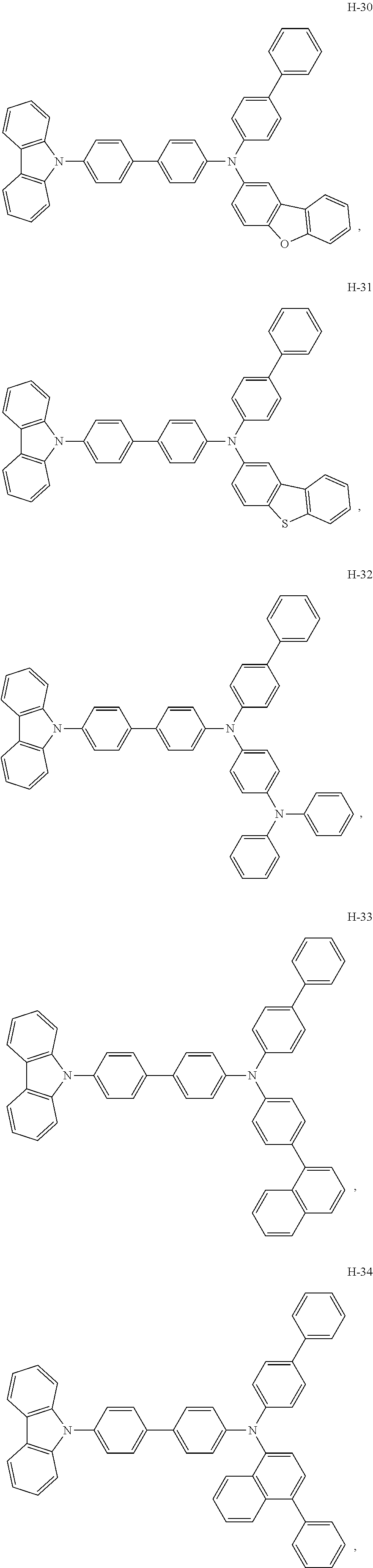 [SMILES: c1ccc(-c2ccc(N(c3ccc(-c4ccc(-n5c6ccccc6c6ccccc65)cc4)cc3)c3ccc4oc5ccccc5c4c3)cc2)cc1 ; c1ccc(-c2ccc(N(c3ccc(-c4ccc(-n5c6ccccc6c6ccccc65)cc4)cc3)c3ccc4sc5ccccc5c4c3)cc2)cc1 ; c1ccc(-c2ccc(N(c3ccc(-c4ccc(-n5c6ccccc6c6ccccc65)cc4)cc3)c3ccc(N(c4ccccc4)c4ccccc4)cc3)cc2)cc1 ; c1ccc(-c2ccc(N(c3ccc(-c4ccc(-n5c6ccccc6c6ccccc65)cc4)cc3)c3ccc(-c4cccc5ccccc45)cc3)cc2)cc1 ; c1ccc(-c2ccc(N(c3ccc(-c4ccc(-n5c6ccccc6c6ccccc65)cc4)cc3)c3ccc(-c4ccccc4)c4ccccc34)cc2)cc1]

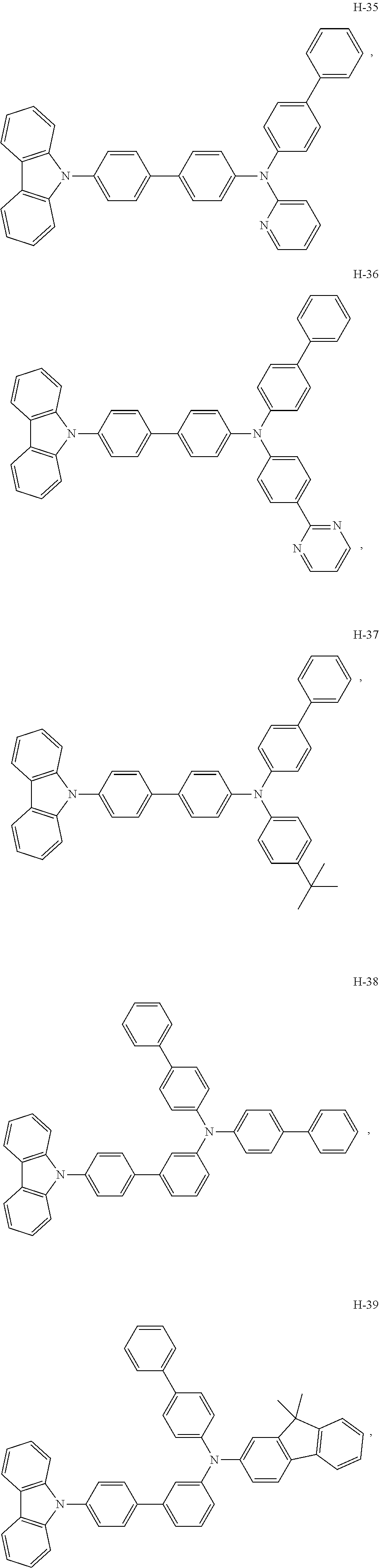 [SMILES: c1ccc(-c2ccc(N(c3ccc(-c4ccc(-n5c6ccccc6c6ccccc65)cc4)cc3)c3ccccn3)cc2)cc1 ; c1ccc(-c2ccc(N(c3ccc(-c4ccc(-n5c6ccccc6c6ccccc65)cc4)cc3)c3ccc(-c4ncccn4)cc3)cc2)cc1 ; CC(C)(C)c1ccc(N(c2ccc(-c3ccccc3)cc2)c2ccc(-c3ccc(-n4c5ccccc5c5ccccc54)cc3)cc2)cc1 ; c1ccc(-c2ccc(N(c3ccc(-c4ccccc4)cc3)c3cccc(-c4ccc(-n5c6ccccc6c6ccccc65)cc4)c3)cc2)cc1 ; CC1(C)c2ccccc2-c2ccc(N(c3ccc(-c4ccccc4)cc3)c3cccc(-c4ccc(-n5c6ccccc6c6ccccc65)cc4)c3)cc21]

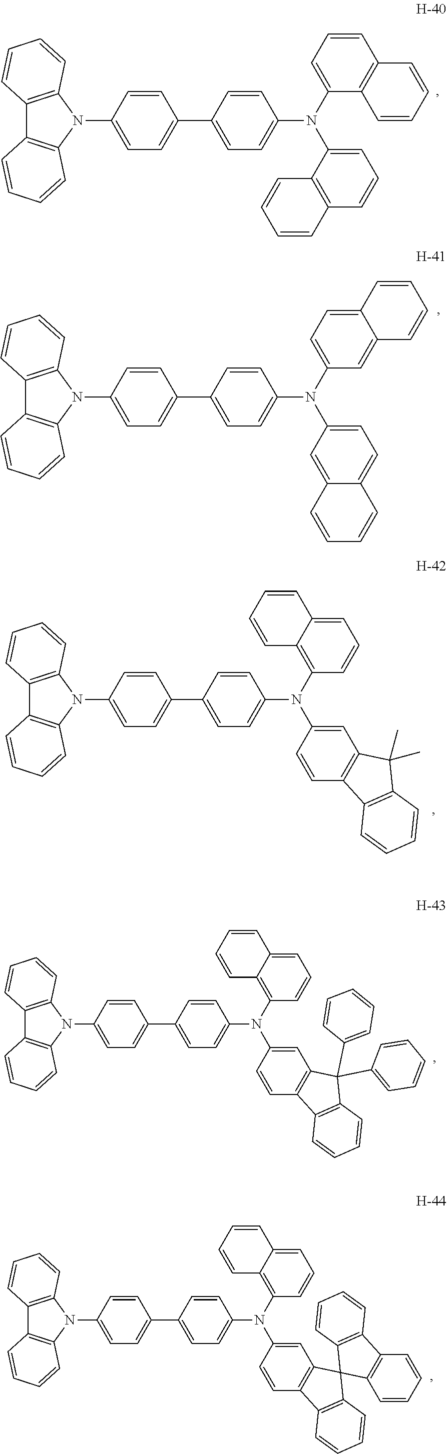 [SMILES: c1ccc2c(N(c3ccc(-c4ccc(-n5c6ccccc6c6ccccc65)cc4)cc3)c3cccc4ccccc34)cccc2c1 ; c1ccc2cc(N(c3ccc(-c4ccc(-n5c6ccccc6c6ccccc65)cc4)cc3)c3ccc4ccccc4c3)ccc2c1 ; CC1(C)c2ccccc2-c2ccc(N(c3ccc(-c4ccc(-n5c6ccccc6c6ccccc65)cc4)cc3)c3cccc4ccccc34)cc21 ; c1ccc(C2(c3ccccc3)c3ccccc3-c3ccc(N(c4ccc(-c5ccc(-n6c7ccccc7c7ccccc76)cc5)cc4)c4cccc5ccccc45)cc32)cc1 ; c1ccc2c(c1)-c1ccccc1C21c2ccccc2-c2ccc(N(c3ccc(-c4ccc(-n5c6ccccc6c6ccccc65)cc4)cc3)c3cccc4ccccc34)cc21]

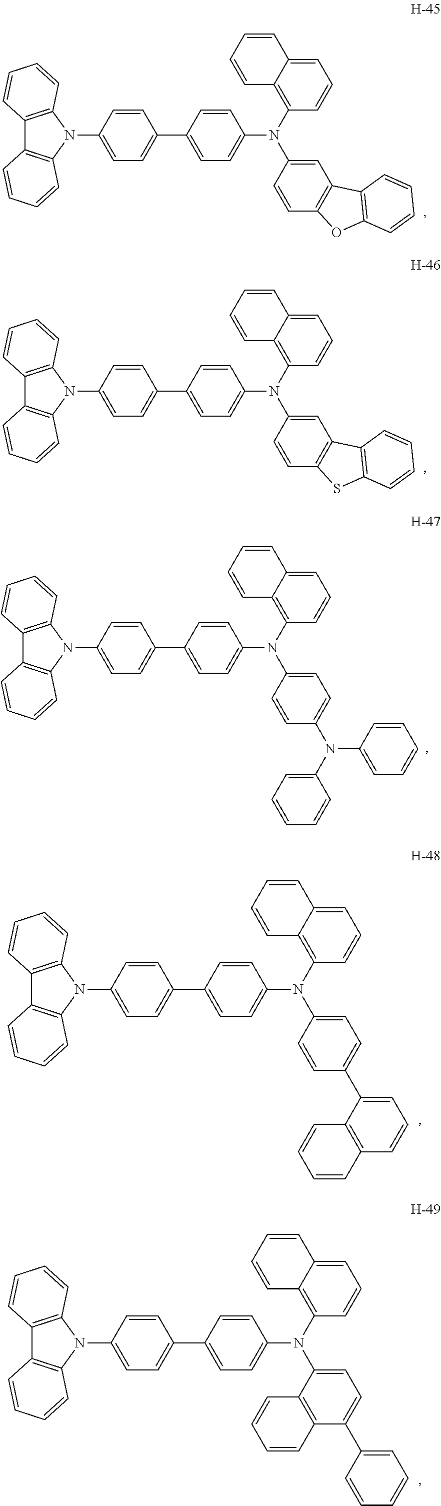 [SMILES: c1ccc2c(N(c3ccc(-c4ccc(-n5c6ccccc6c6ccccc65)cc4)cc3)c3ccc4oc5ccccc5c4c3)cccc2c1 ; c1ccc2c(N(c3ccc(-c4ccc(-n5c6ccccc6c6ccccc65)cc4)cc3)c3ccc4sc5ccccc5c4c3)cccc2c1 ; c1ccc(N(c2ccccc2)c2ccc(N(c3ccc(-c4ccc(-n5c6ccccc6c6ccccc65)cc4)cc3)c3cccc4ccccc34)cc2)cc1 ; c1ccc2c(-c3ccc(N(c4ccc(-c5ccc(-n6c7ccccc7c7ccccc76)cc5)cc4)c4cccc5ccccc45)cc3)cccc2c1 ; c1ccc(-c2ccc(N(c3ccc(-c4ccc(-n5c6ccccc6c6ccccc65)cc4)cc3)c3cccc4ccccc34)c3ccccc23)cc1]

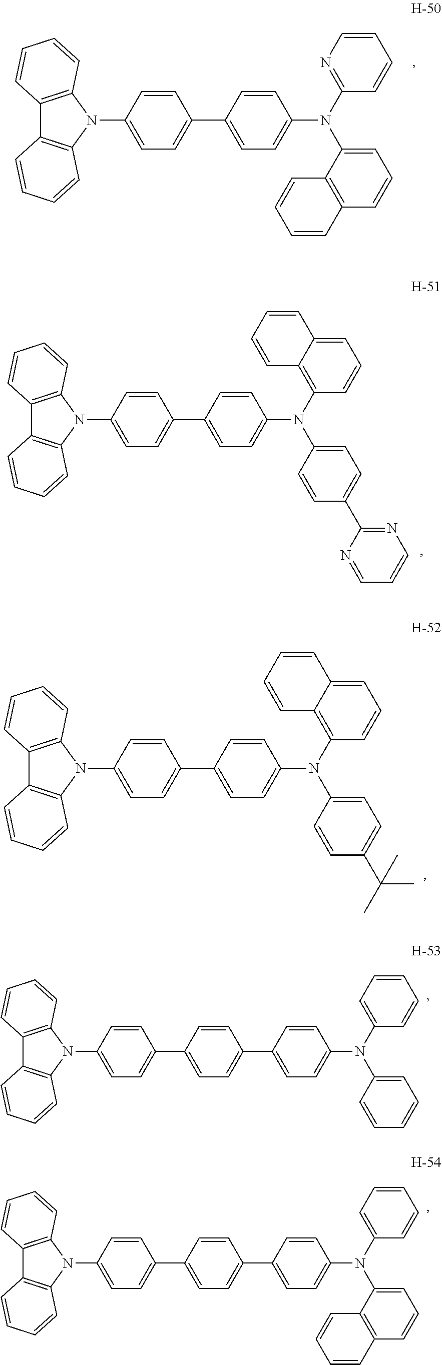 [SMILES: c1ccc(N(c2ccc(-c3ccc(-n4c5ccccc5c5ccccc54)cc3)cc2)c2cccc3ccccc23)nc1 ; c1cnc(-c2ccc(N(c3ccc(-c4ccc(-n5c6ccccc6c6ccccc65)cc4)cc3)c3cccc4ccccc34)cc2)nc1 ; CC(C)(C)c1ccc(N(c2ccc(-c3ccc(-n4c5ccccc5c5ccccc54)cc3)cc2)c2cccc3ccccc23)cc1 ; c1ccc(N(c2ccccc2)c2ccc(-c3ccc(-c4ccc(-n5c6ccccc6c6ccccc65)cc4)cc3)cc2)cc1 ; c1ccc(N(c2ccc(-c3ccc(-c4ccc(-n5c6ccccc6c6ccccc65)cc4)cc3)cc2)c2cccc3ccccc23)cc1]

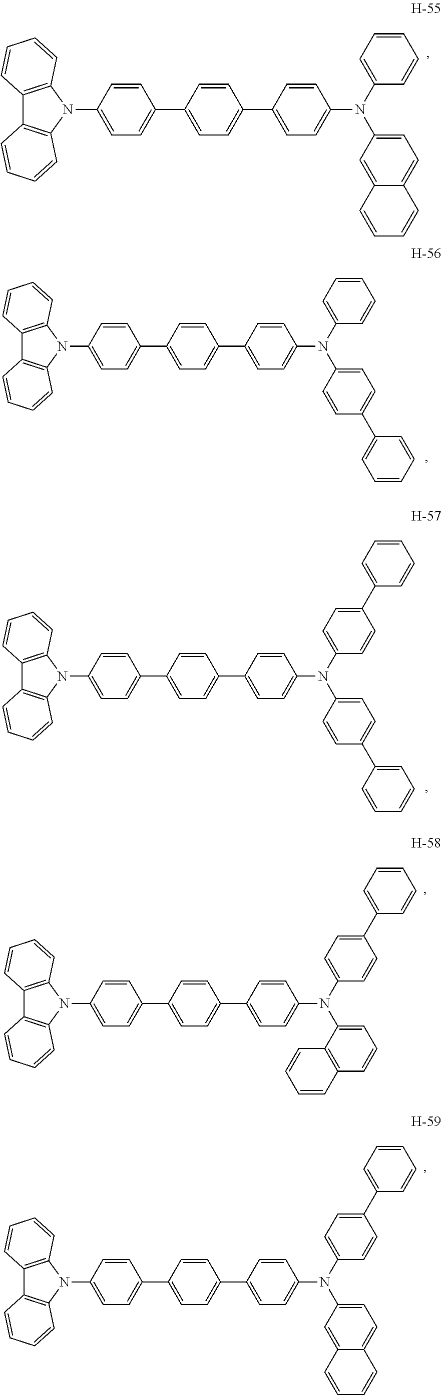 [SMILES: c1ccc(N(c2ccc(-c3ccc(-c4ccc(-n5c6ccccc6c6ccccc65)cc4)cc3)cc2)c2ccc3ccccc3c2)cc1 ; c1ccc(-c2ccc(N(c3ccccc3)c3ccc(-c4ccc(-c5ccc(-n6c7ccccc7c7ccccc76)cc5)cc4)cc3)cc2)cc1 ; c1ccc(-c2ccc(N(c3ccc(-c4ccccc4)cc3)c3ccc(-c4ccc(-c5ccc(-n6c7ccccc7c7ccccc76)cc5)cc4)cc3)cc2)cc1 ; c1ccc(-c2ccc(N(c3ccc(-c4ccc(-c5ccc(-n6c7ccccc7c7ccccc76)cc5)cc4)cc3)c3cccc4ccccc34)cc2)cc1 ; c1ccc(-c2ccc(N(c3ccc(-c4ccc(-c5ccc(-n6c7ccccc7c7ccccc76)cc5)cc4)cc3)c3ccc4ccccc4c3)cc2)cc1]

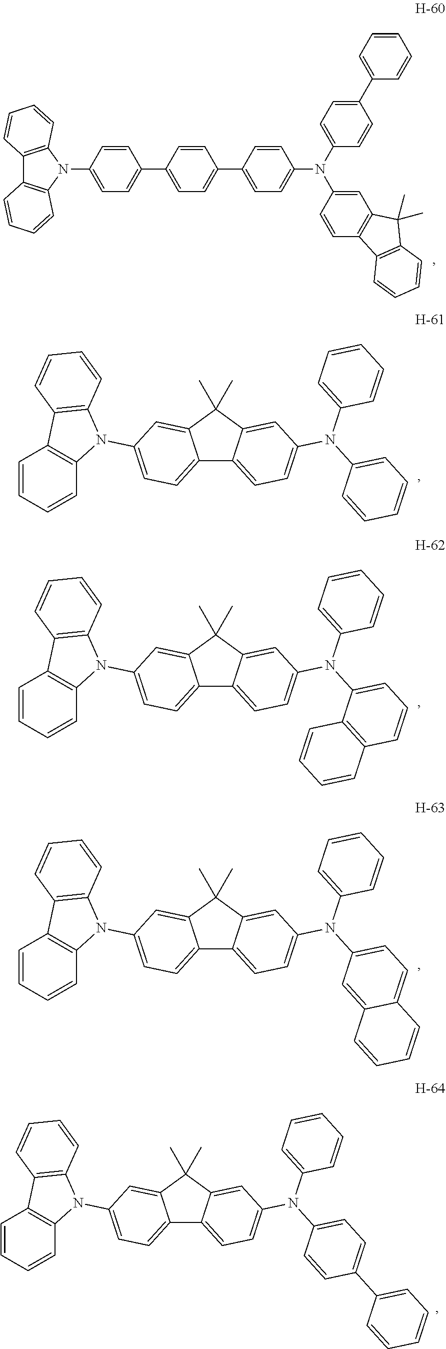 [SMILES: CC1(C)c2ccccc2-c2ccc(N(c3ccc(-c4ccccc4)cc3)c3ccc(-c4ccc(-c5ccc(-n6c7ccccc7c7ccccc76)cc5)cc4)cc3)cc21 ; CC1(C)c2cc(N(c3ccccc3)c3ccccc3)ccc2-c2ccc(-n3c4ccccc4c4ccccc43)cc21 ; CC1(C)c2cc(N(c3ccccc3)c3cccc4ccccc34)ccc2-c2ccc(-n3c4ccccc4c4ccccc43)cc21 ; CC1(C)c2cc(N(c3ccccc3)c3ccc4ccccc4c3)ccc2-c2ccc(-n3c4ccccc4c4ccccc43)cc21 ; CC1(C)c2cc(N(c3ccccc3)c3ccc(-c4ccccc4)cc3)ccc2-c2ccc(-n3c4ccccc4c4ccccc43)cc21]

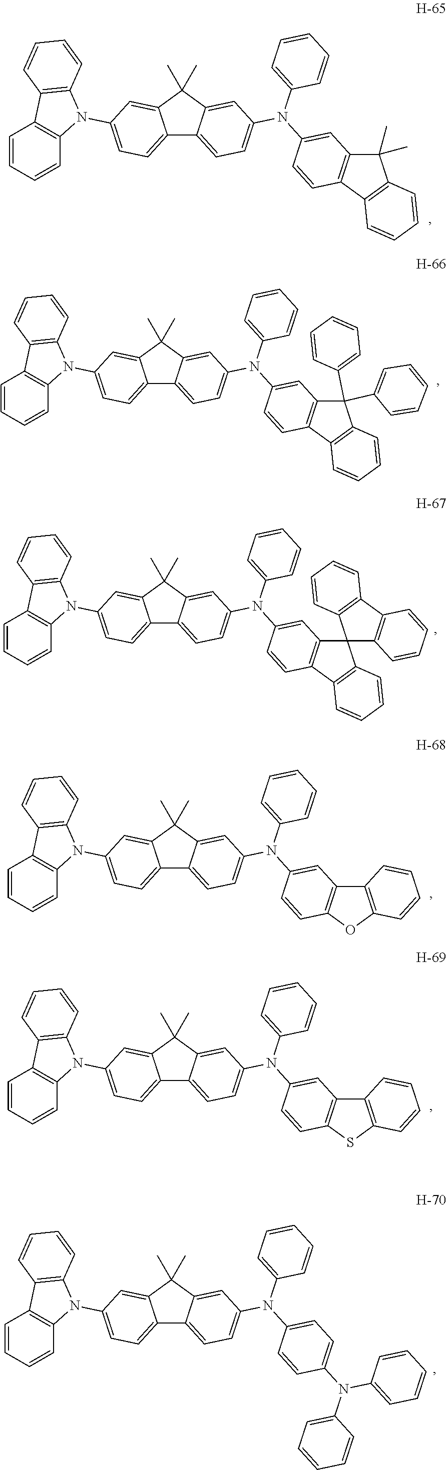

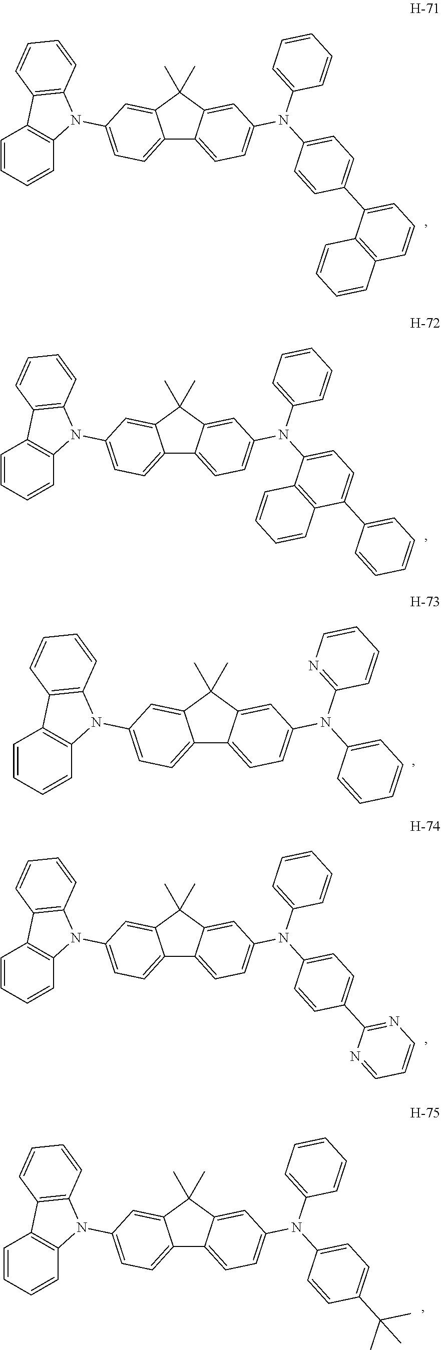 [SMILES: CC1(C)c2cc(N(c3ccccc3)c3ccc(-c4cccc5ccccc45)cc3)ccc2-c2ccc(-n3c4ccccc4c4ccccc43)cc21 ; CC1(C)c2cc(N(c3ccccc3)c3ccc(-c4ccccc4)c4ccccc34)ccc2-c2ccc(-n3c4ccccc4c4ccccc43)cc21 ; CC1(C)c2cc(N(c3ccccc3)c3ccccn3)ccc2-c2ccc(-n3c4ccccc4c4ccccc43)cc21 ; CC1(C)c2cc(N(c3ccccc3)c3ccc(-c4ncccn4)cc3)ccc2-c2ccc(-n3c4ccccc4c4ccccc43)cc21 ; CC(C)(C)c1ccc(N(c2ccccc2)c2ccc3c(c2)C(C)(C)c2cc(-n4c5ccccc5c5ccccc54)ccc2-3)cc1]

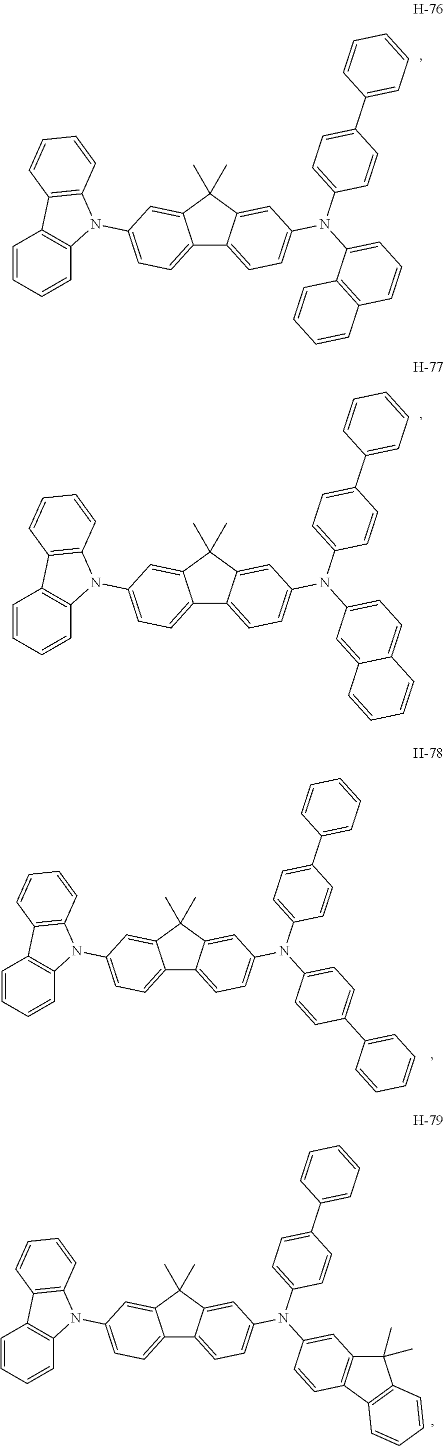 [SMILES: CC1(C)c2cc(N(c3ccc(-c4ccccc4)cc3)c3cccc4ccccc34)ccc2-c2ccc(-n3c4ccccc4c4ccccc43)cc21 ; CC1(C)c2cc(N(c3ccc(-c4ccccc4)cc3)c3ccc4ccccc4c3)ccc2-c2ccc(-n3c4ccccc4c4ccccc43)cc21 ; CC1(C)c2cc(N(c3ccc(-c4ccccc4)cc3)c3ccc(-c4ccccc4)cc3)ccc2-c2ccc(-n3c4ccccc4c4ccccc43)cc21 ; CC1(C)c2ccccc2-c2ccc(N(c3ccc(-c4ccccc4)cc3)c3ccc4c(c3)C(C)(C)c3cc(-n5c6ccccc6c6ccccc65)ccc3-4)cc21]

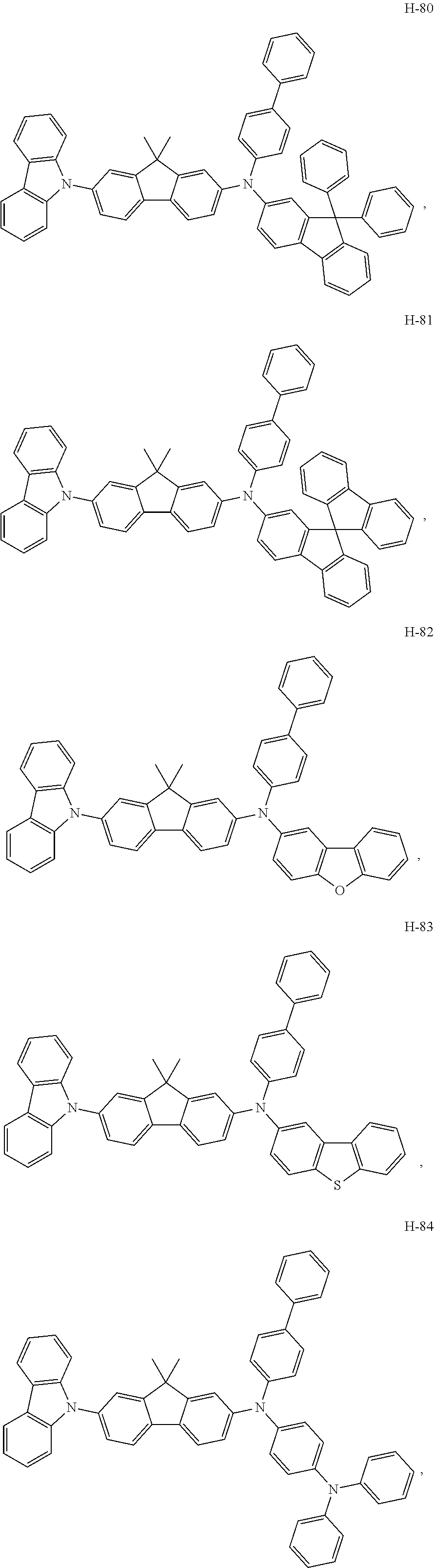

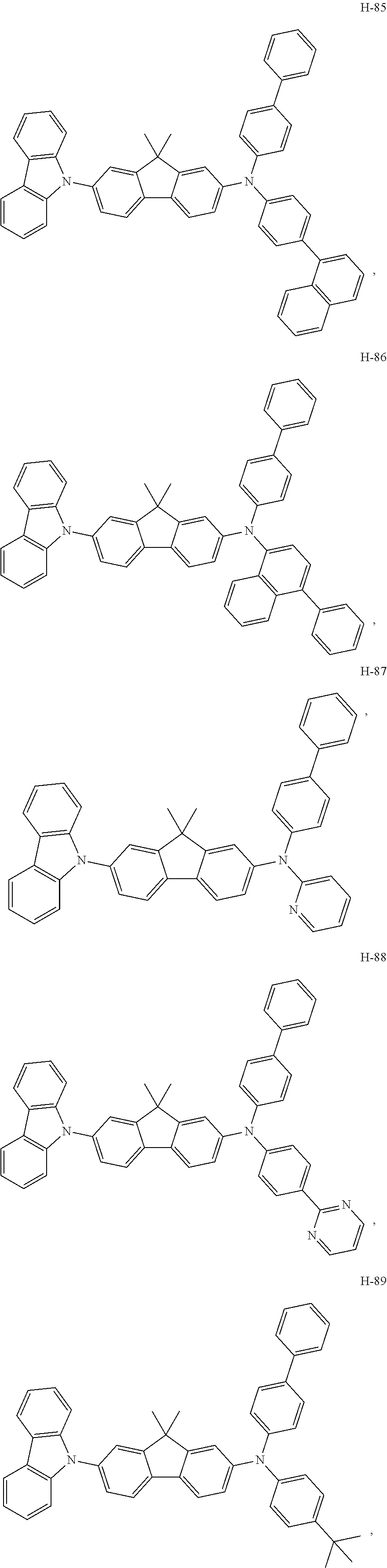 [SMILES: CC1(C)c2cc(N(c3ccc(-c4ccccc4)cc3)c3ccc(-c4cccc5ccccc45)cc3)ccc2-c2ccc(-n3c4ccccc4c4ccccc43)cc21 ; CC1(C)c2cc(N(c3ccc(-c4ccccc4)cc3)c3ccc(-c4ccccc4)c4ccccc34)ccc2-c2ccc(-n3c4ccccc4c4ccccc43)cc21 ; CC1(C)c2cc(N(c3ccc(-c4ccccc4)cc3)c3ccccn3)ccc2-c2ccc(-n3c4ccccc4c4ccccc43)cc21 ; CC1(C)c2cc(N(c3ccc(-c4ccccc4)cc3)c3ccc(-c4ncccn4)cc3)ccc2-c2ccc(-n3c4ccccc4c4ccccc43)cc21 ; CC(C)(C)c1ccc(N(c2ccc(-c3ccccc3)cc2)c2ccc3c(c2)C(C)(C)c2cc(-n4c5ccccc5c5ccccc54)ccc2-3)cc1]

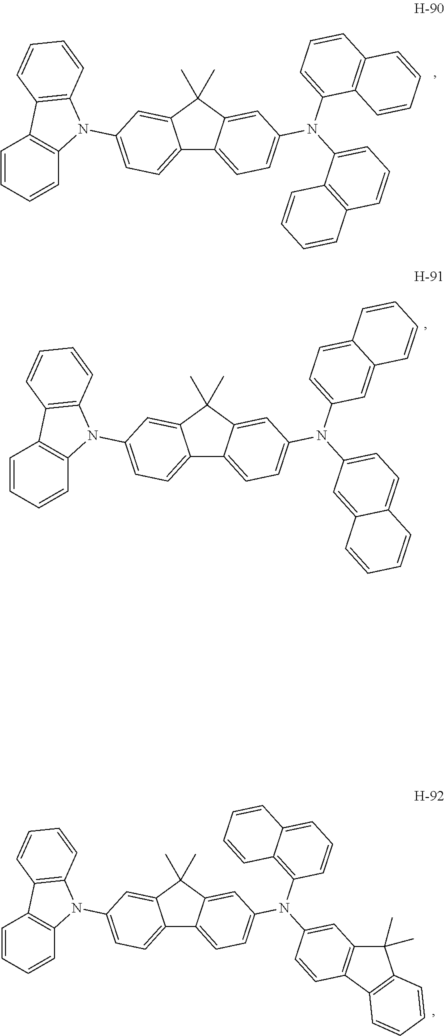 [SMILES: CC1(C)c2cc(N(c3cccc4ccccc34)c3cccc4ccccc34)ccc2-c2ccc(-n3c4ccccc4c4ccccc43)cc21 ; CC1(C)c2cc(N(c3ccc4ccccc4c3)c3ccc4ccccc4c3)ccc2-c2ccc(-n3c4ccccc4c4ccccc43)cc21 ; CC1(C)c2ccccc2-c2ccc(N(c3ccc4c(c3)C(C)(C)c3cc(-n5c6ccccc6c6ccccc65)ccc3-4)c3cccc4ccccc34)cc21]

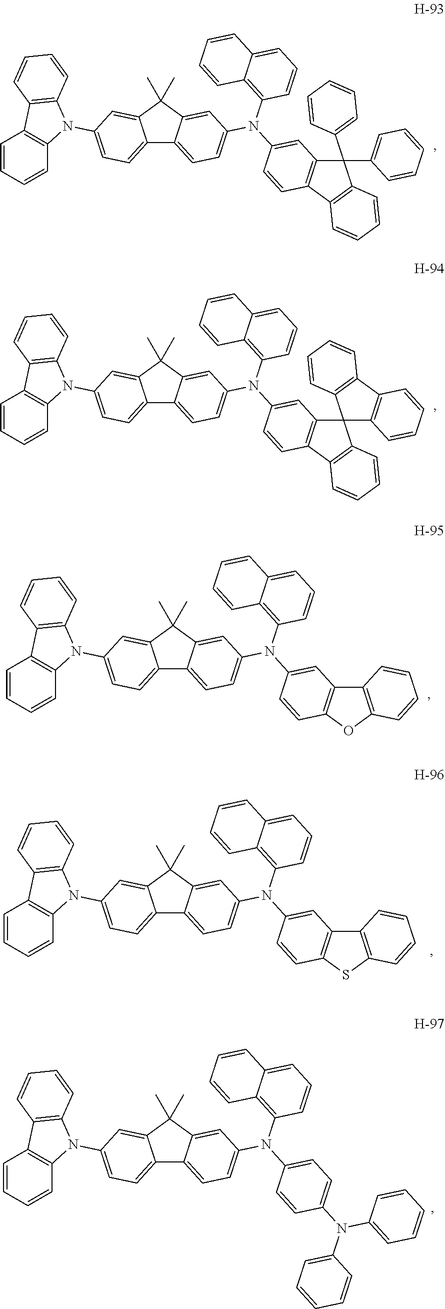 [SMILES: CC1(C)c2cc(N(c3ccc4c(c3)C(c3ccccc3)(c3ccccc3)c3ccccc3-4)c3cccc4ccccc34)ccc2-c2ccc(-n3c4ccccc4c4ccccc43)cc21 ; CC1(C)c2cc(N(c3ccc4c(c3)C3(c5ccccc5-c5ccccc53)c3ccccc3-4)c3cccc4ccccc34)ccc2-c2ccc(-n3c4ccccc4c4ccccc43)cc21 ; CC1(C)c2cc(N(c3ccc4oc5ccccc5c4c3)c3cccc4ccccc34)ccc2-c2ccc(-n3c4ccccc4c4ccccc43)cc21 ; CC1(C)c2cc(N(c3ccc4sc5ccccc5c4c3)c3cccc4ccccc34)ccc2-c2ccc(-n3c4ccccc4c4ccccc43)cc21 ; CC1(C)c2cc(N(c3ccc(N(c4ccccc4)c4ccccc4)cc3)c3cccc4ccccc34)ccc2-c2ccc(-n3c4ccccc4c4ccccc43)cc21]

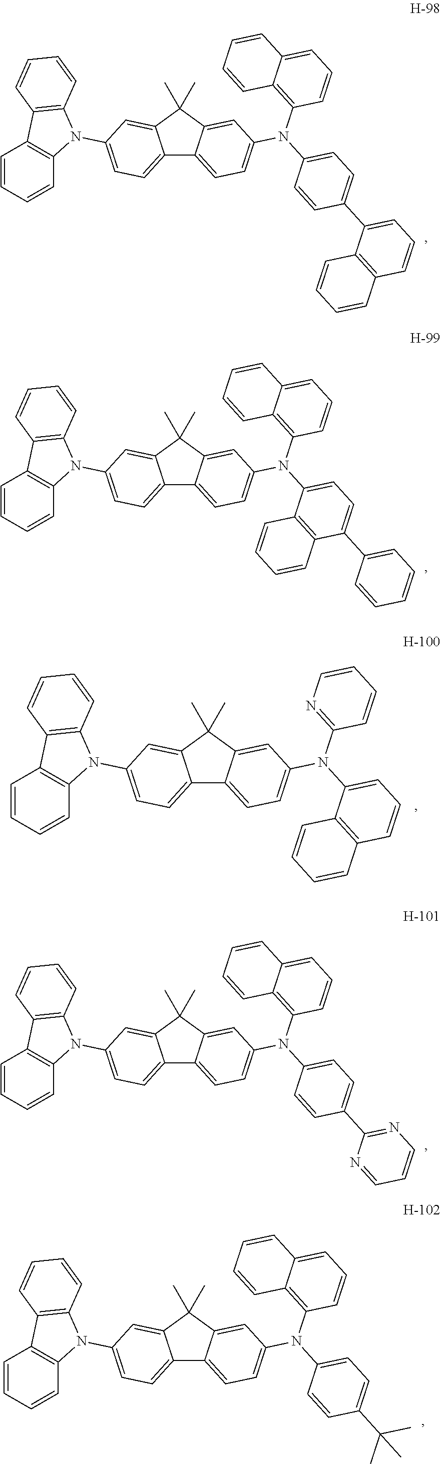 [SMILES: CC1(C)c2cc(N(c3ccc(-c4cccc5ccccc45)cc3)c3cccc4ccccc34)ccc2-c2ccc(-n3c4ccccc4c4ccccc43)cc21 ; CC1(C)c2cc(N(c3cccc4ccccc34)c3ccc(-c4ccccc4)c4ccccc34)ccc2-c2ccc(-n3c4ccccc4c4ccccc43)cc21 ; CC1(C)c2cc(N(c3ccccn3)c3cccc4ccccc34)ccc2-c2ccc(-n3c4ccccc4c4ccccc43)cc21 ; CC1(C)c2cc(N(c3ccc(-c4ncccn4)cc3)c3cccc4ccccc34)ccc2-c2ccc(-n3c4ccccc4c4ccccc43)cc21 ; CC(C)(C)c1ccc(N(c2ccc3c(c2)C(C)(C)c2cc(-n4c5ccccc5c5ccccc54)ccc2-3)c2cccc3ccccc23)cc1]

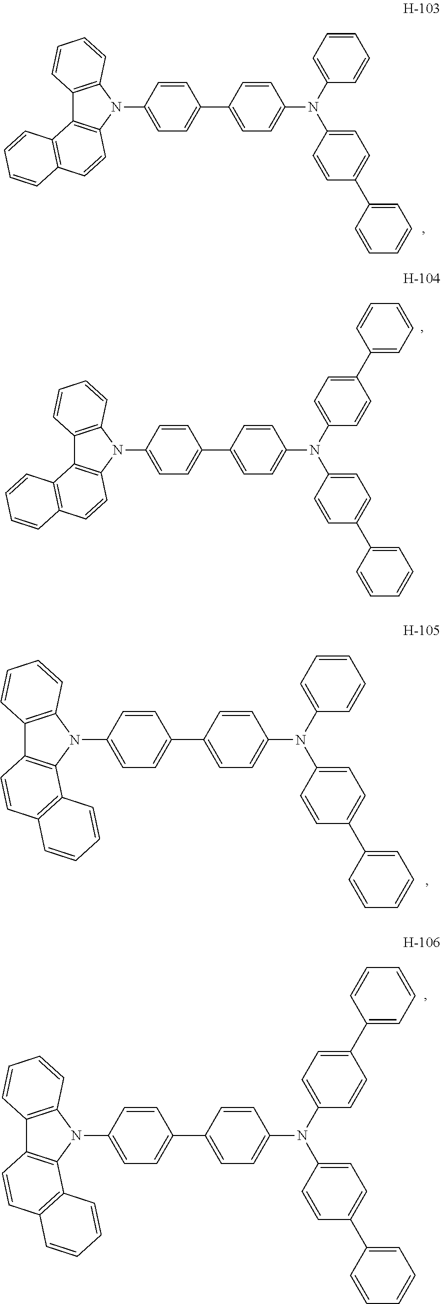 [SMILES: c1ccc(-c2ccc(N(c3ccccc3)c3ccc(-c4ccc(-n5c6ccccc6c6c7ccccc7ccc65)cc4)cc3)cc2)cc1 ; c1ccc(-c2ccc(N(c3ccc(-c4ccccc4)cc3)c3ccc(-c4ccc(-n5c6ccccc6c6c7ccccc7ccc65)cc4)cc3)cc2)cc1 ; c1ccc(-c2ccc(N(c3ccccc3)c3ccc(-c4ccc(-n5c6ccccc6c6ccc7ccccc7c65)cc4)cc3)cc2)cc1 ; c1ccc(-c2ccc(N(c3ccc(-c4ccccc4)cc3)c3ccc(-c4ccc(-n5c6ccccc6c6ccc7ccccc7c65)cc4)cc3)cc2)cc1]

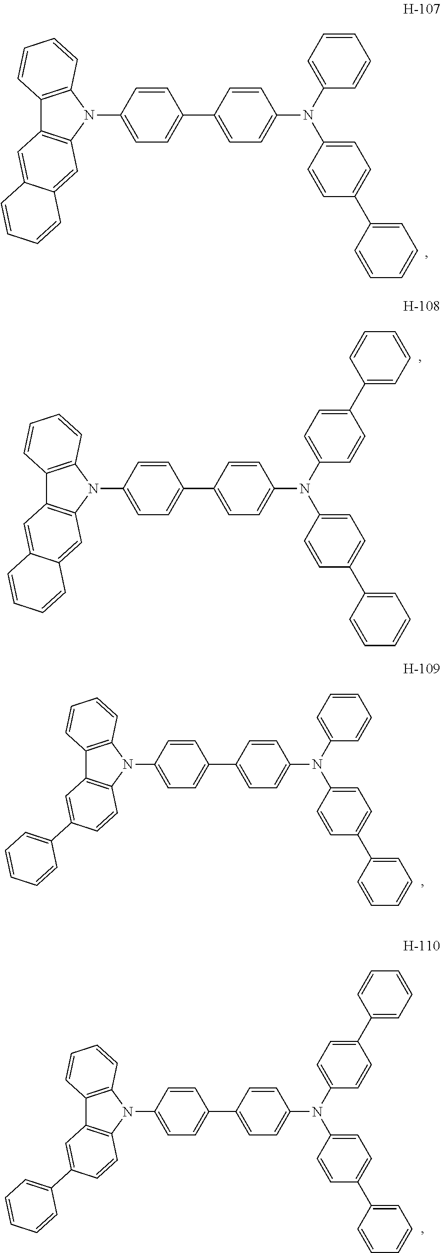 [SMILES: c1ccc(-c2ccc(N(c3ccccc3)c3ccc(-c4ccc(-n5c6ccccc6c6cc7ccccc7cc65)cc4)cc3)cc2)cc1 ; c1ccc(-c2ccc(N(c3ccc(-c4ccccc4)cc3)c3ccc(-c4ccc(-n5c6ccccc6c6cc7ccccc7cc65)cc4)cc3)cc2)cc1 ; c1ccc(-c2ccc(N(c3ccccc3)c3ccc(-c4ccc(-n5c6ccccc6c6cc(-c7ccccc7)ccc65)cc4)cc3)cc2)cc1 ; c1ccc(-c2ccc(N(c3ccc(-c4ccccc4)cc3)c3ccc(-c4ccc(-n5c6ccccc6c6cc(-c7ccccc7)ccc65)cc4)cc3)cc2)cc1]

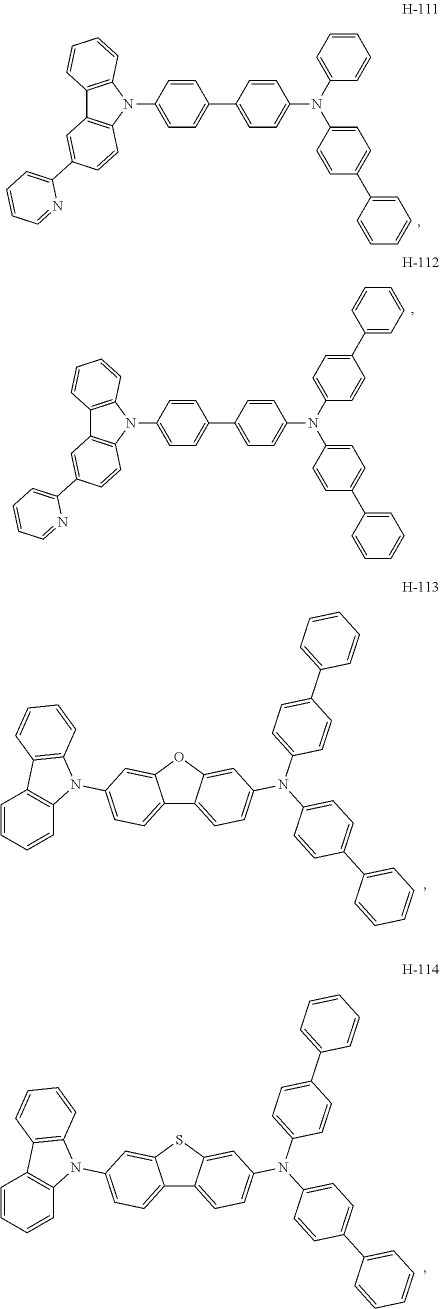 [SMILES: c1ccc(-c2ccc(N(c3ccccc3)c3ccc(-c4ccc(-n5c6ccccc6c6cc(-c7ccccn7)ccc65)cc4)cc3)cc2)cc1 ; c1ccc(-c2ccc(N(c3ccc(-c4ccccc4)cc3)c3ccc(-c4ccc(-n5c6ccccc6c6cc(-c7ccccn7)ccc65)cc4)cc3)cc2)cc1 ; c1ccc(-c2ccc(N(c3ccc(-c4ccccc4)cc3)c3ccc4c(c3)oc3cc(-n5c6ccccc6c6ccccc65)ccc34)cc2)cc1 ; c1ccc(-c2ccc(N(c3ccc(-c4ccccc4)cc3)c3ccc4c(c3)sc3cc(-n5c6ccccc6c6ccccc65)ccc34)cc2)cc1]

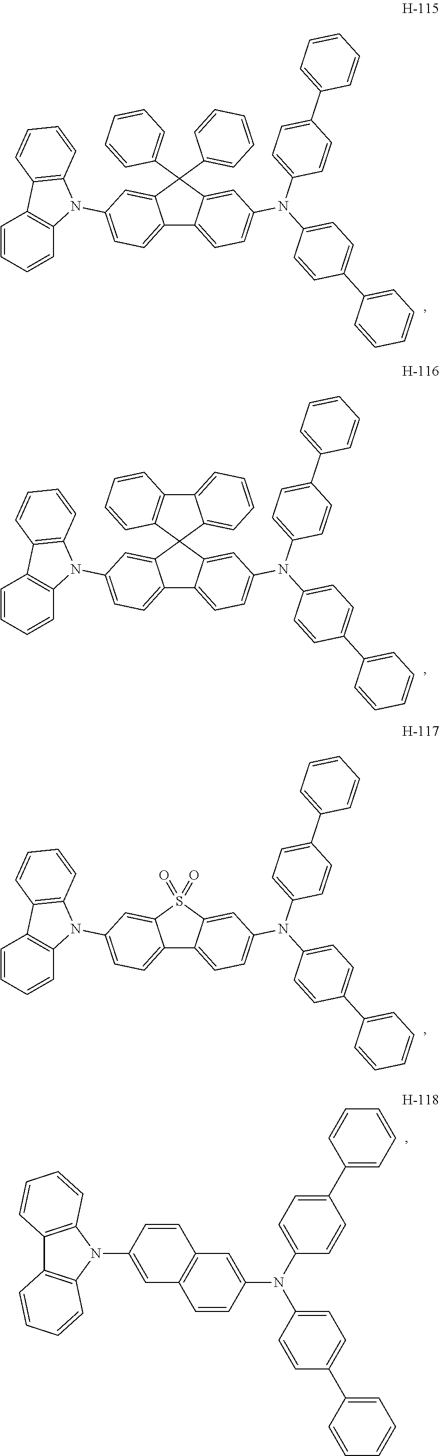

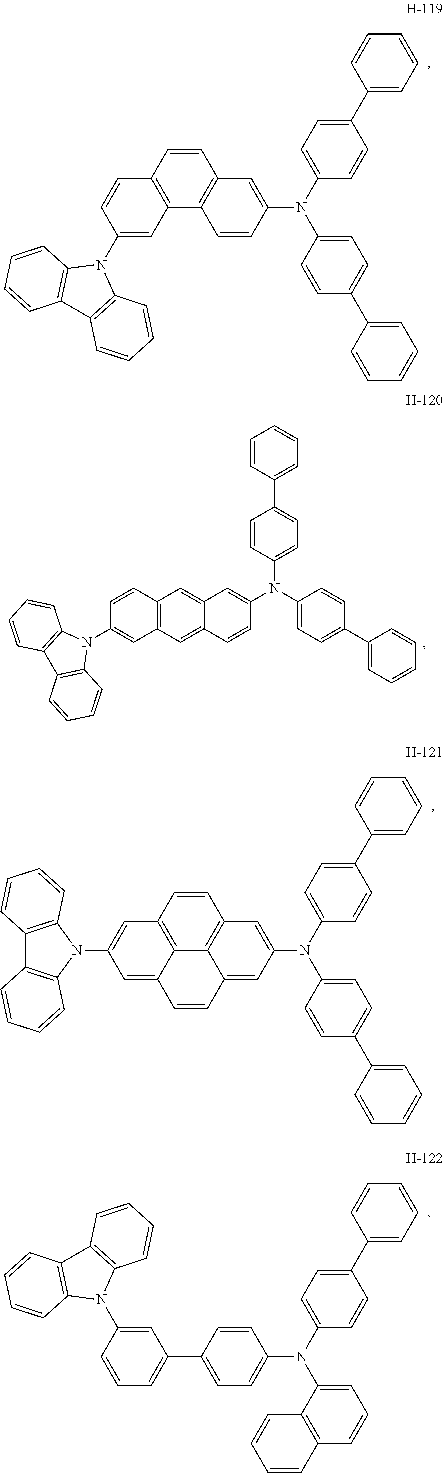 [SMILES: c1ccc(-c2ccc(N(c3ccc(-c4ccccc4)cc3)c3ccc4c(ccc5ccc(-n6c7ccccc7c7ccccc76)cc54)c3)cc2)cc1 ; c1ccc(-c2ccc(N(c3ccc(-c4ccccc4)cc3)c3ccc4cc5cc(-n6c7ccccc7c7ccccc76)ccc5cc4c3)cc2)cc1 ; c1ccc(-c2ccc(N(c3ccc(-c4ccccc4)cc3)c3cc4ccc5cc(-n6c7ccccc7c7ccccc76)cc6ccc(c3)c4c56)cc2)cc1 ; c1ccc(-c2ccc(N(c3ccc(-c4cccc(-n5c6ccccc6c6ccccc65)c4)cc3)c3cccc4ccccc34)cc2)cc1]

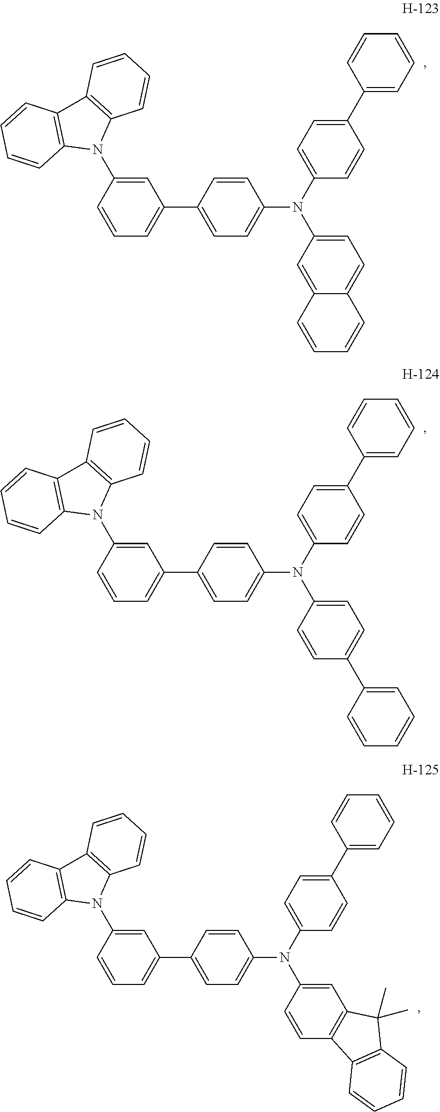 [SMILES: c1ccc(-c2ccc(N(c3ccc(-c4cccc(-n5c6ccccc6c6ccccc65)c4)cc3)c3ccc4ccccc4c3)cc2)cc1 ; c1ccc(-c2ccc(N(c3ccc(-c4ccccc4)cc3)c3ccc(-c4cccc(-n5c6ccccc6c6ccccc65)c4)cc3)cc2)cc1 ; CC1(C)c2ccccc2-c2ccc(N(c3ccc(-c4ccccc4)cc3)c3ccc(-c4cccc(-n5c6ccccc6c6ccccc65)c4)cc3)cc21]

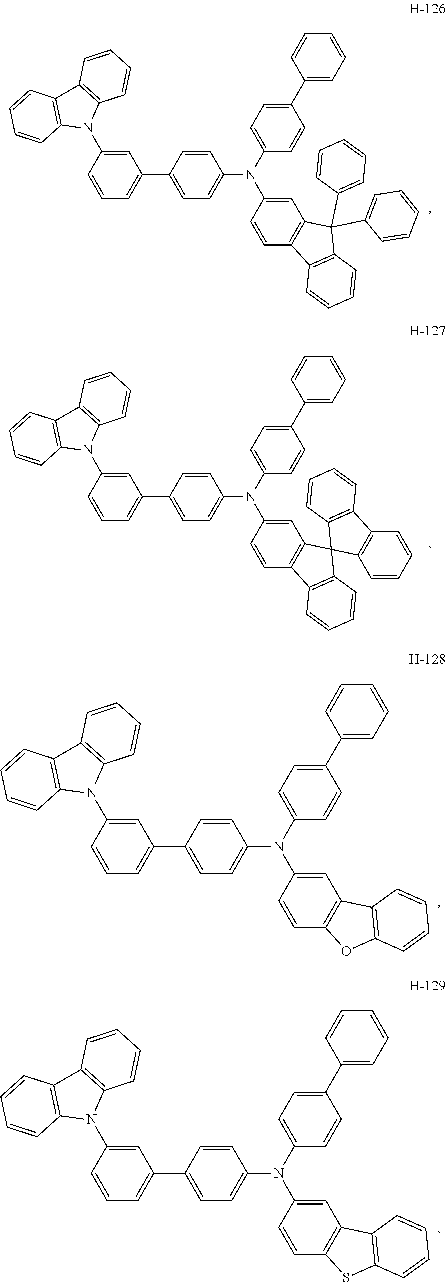 [SMILES: c1ccc(-c2ccc(N(c3ccc(-c4cccc(-n5c6ccccc6c6ccccc65)c4)cc3)c3ccc4c(c3)C(c3ccccc3)(c3ccccc3)c3ccccc3-4)cc2)cc1 ; c1ccc(-c2ccc(N(c3ccc(-c4cccc(-n5c6ccccc6c6ccccc65)c4)cc3)c3ccc4c(c3)C3(c5ccccc5-c5ccccc53)c3ccccc3-4)cc2)cc1 ; c1ccc(-c2ccc(N(c3ccc(-c4cccc(-n5c6ccccc6c6ccccc65)c4)cc3)c3ccc4oc5ccccc5c4c3)cc2)cc1 ; c1ccc(-c2ccc(N(c3ccc(-c4cccc(-n5c6ccccc6c6ccccc65)c4)cc3)c3ccc4sc5ccccc5c4c3)cc2)cc1]

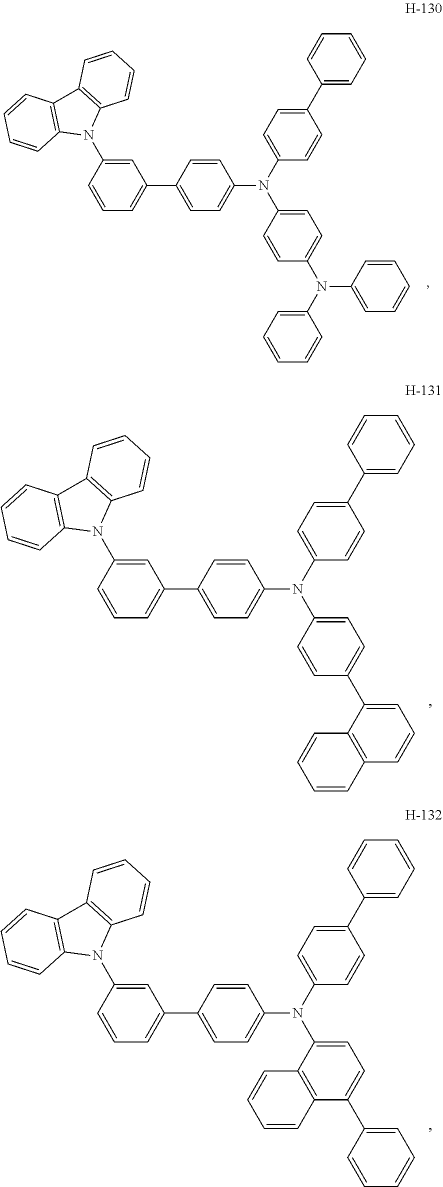 [SMILES: c1ccc(-c2ccc(N(c3ccc(-c4cccc(-n5c6ccccc6c6ccccc65)c4)cc3)c3ccc(N(c4ccccc4)c4ccccc4)cc3)cc2)cc1 ; c1ccc(-c2ccc(N(c3ccc(-c4cccc(-n5c6ccccc6c6ccccc65)c4)cc3)c3ccc(-c4cccc5ccccc45)cc3)cc2)cc1 ; c1ccc(-c2ccc(N(c3ccc(-c4cccc(-n5c6ccccc6c6ccccc65)c4)cc3)c3ccc(-c4ccccc4)c4ccccc34)cc2)cc1]

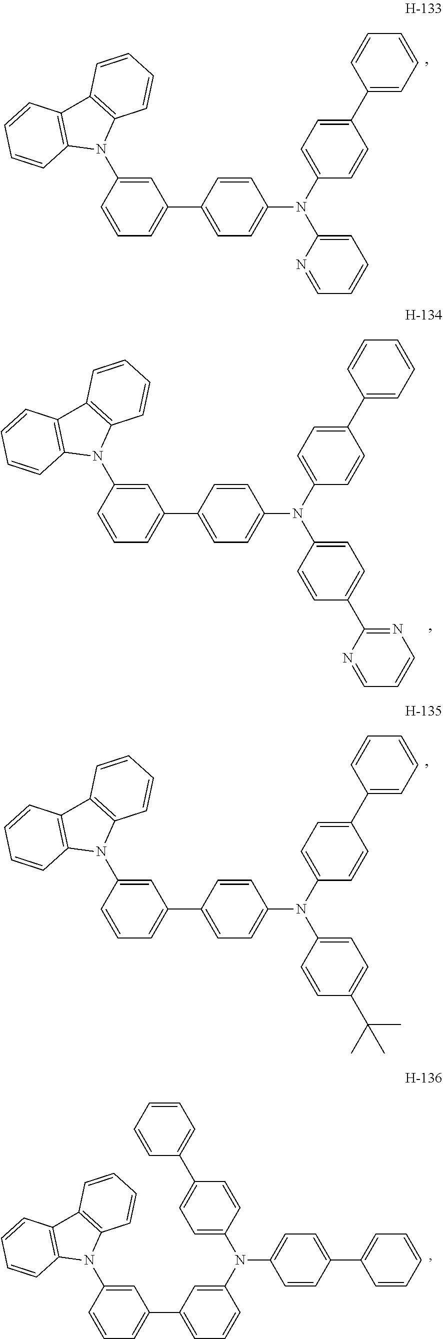 [SMILES: c1ccc(-c2ccc(N(c3ccc(-c4cccc(-n5c6ccccc6c6ccccc65)c4)cc3)c3ccccn3)cc2)cc1 ; c1ccc(-c2ccc(N(c3ccc(-c4cccc(-n5c6ccccc6c6ccccc65)c4)cc3)c3ccc(-c4ncccn4)cc3)cc2)cc1 ; CC(C)(C)c1ccc(N(c2ccc(-c3ccccc3)cc2)c2ccc(-c3cccc(-n4c5ccccc5c5ccccc54)c3)cc2)cc1 ; c1ccc(-c2ccc(N(c3ccc(-c4ccccc4)cc3)c3cccc(-c4cccc(-n5c6ccccc6c6ccccc65)c4)c3)cc2)cc1]

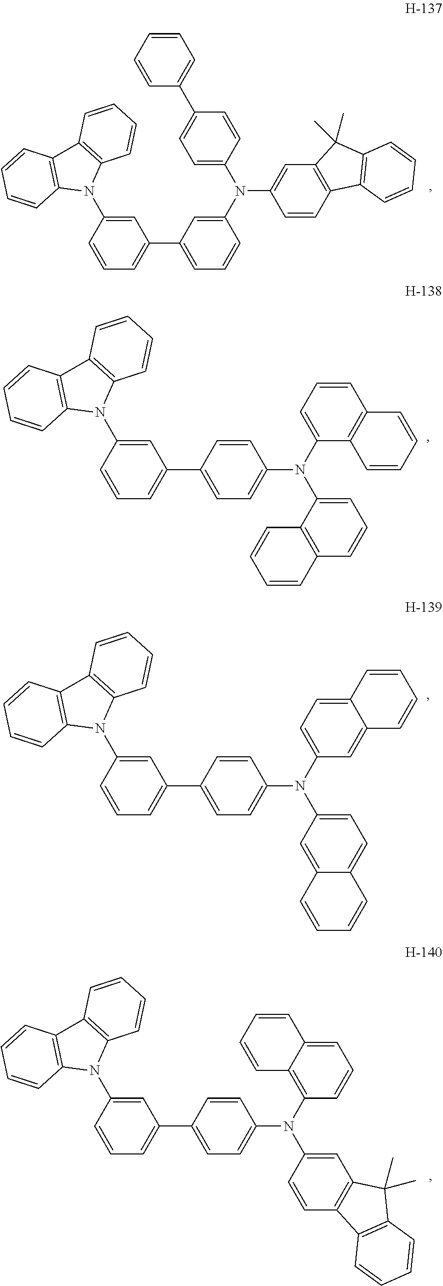 [SMILES: CC1(C)c2ccccc2-c2ccc(N(c3ccc(-c4ccccc4)cc3)c3cccc(-c4cccc(-n5c6ccccc6c6ccccc65)c4)c3)cc21 ; c1cc(-c2ccc(N(c3cccc4ccccc34)c3cccc4ccccc34)cc2)cc(-n2c3ccccc3c3ccccc32)c1 ; c1cc(-c2ccc(N(c3ccc4ccccc4c3)c3ccc4ccccc4c3)cc2)cc(-n2c3ccccc3c3ccccc32)c1 ; CC1(C)c2ccccc2-c2ccc(N(c3ccc(-c4cccc(-n5c6ccccc6c6ccccc65)c4)cc3)c3cccc4ccccc34)cc21]

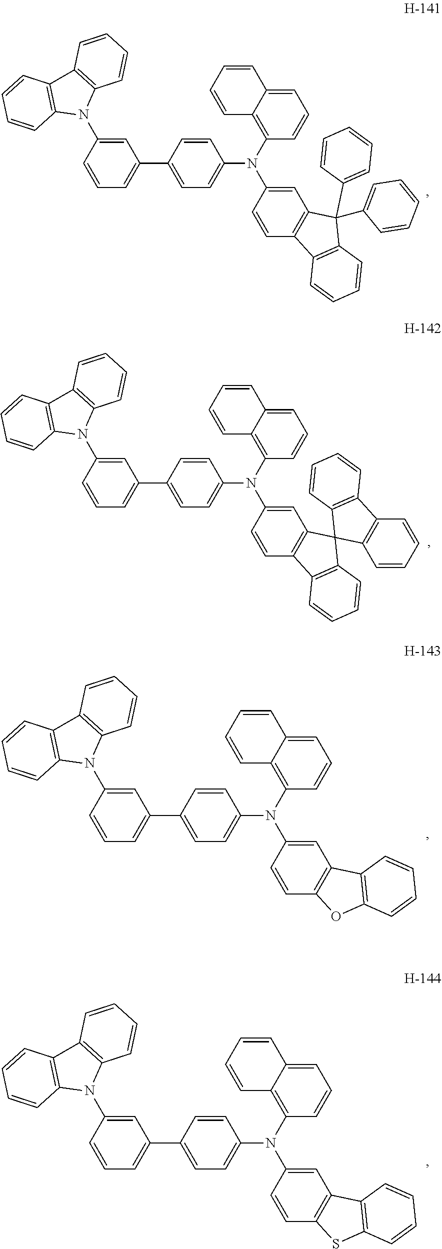 [SMILES: c1ccc(C2(c3ccccc3)c3ccccc3-c3ccc(N(c4ccc(-c5cccc(-n6c7ccccc7c7ccccc76)c5)cc4)c4cccc5ccccc45)cc32)cc1 ; c1cc(-c2ccc(N(c3ccc4c(c3)C3(c5ccccc5-c5ccccc53)c3ccccc3-4)c3cccc4ccccc34)cc2)cc(-n2c3ccccc3c3ccccc32)c1 ; c1cc(-c2ccc(N(c3ccc4oc5ccccc5c4c3)c3cccc4ccccc34)cc2)cc(-n2c3ccccc3c3ccccc32)c1 ; c1cc(-c2ccc(N(c3ccc4sc5ccccc5c4c3)c3cccc4ccccc34)cc2)cc(-n2c3ccccc3c3ccccc32)c1]

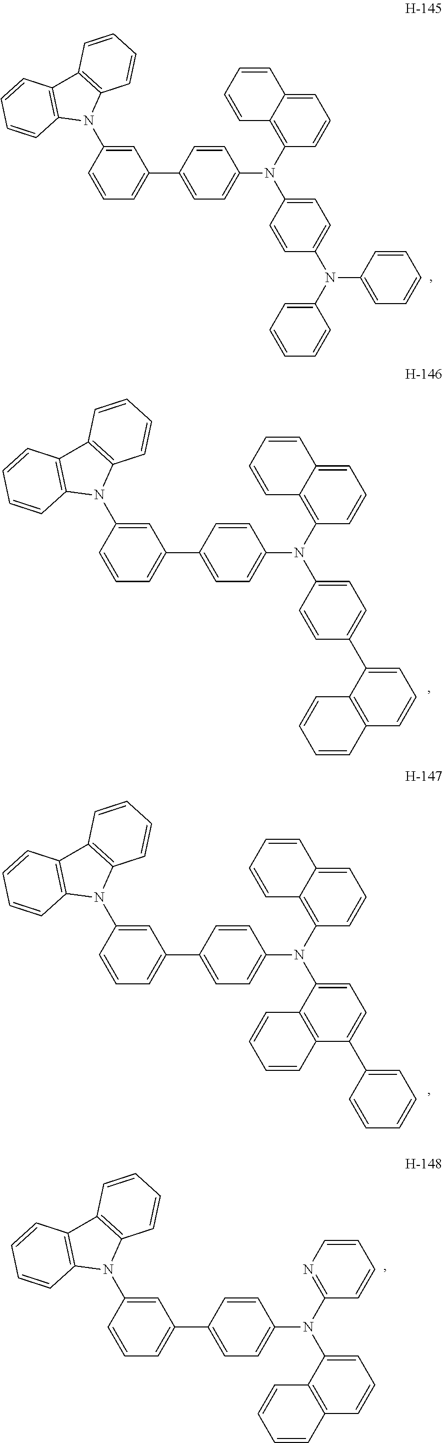 [SMILES: c1ccc(N(c2ccccc2)c2ccc(N(c3ccc(-c4cccc(-n5c6ccccc6c6ccccc65)c4)cc3)c3cccc4ccccc34)cc2)cc1 ; c1cc(-c2ccc(N(c3ccc(-c4cccc5ccccc45)cc3)c3cccc4ccccc34)cc2)cc(-n2c3ccccc3c3ccccc32)c1 ; c1ccc(-c2ccc(N(c3ccc(-c4cccc(-n5c6ccccc6c6ccccc65)c4)cc3)c3cccc4ccccc34)c3ccccc23)cc1 ; c1ccc(N(c2ccc(-c3cccc(-n4c5ccccc5c5ccccc54)c3)cc2)c2cccc3ccccc23)nc1]

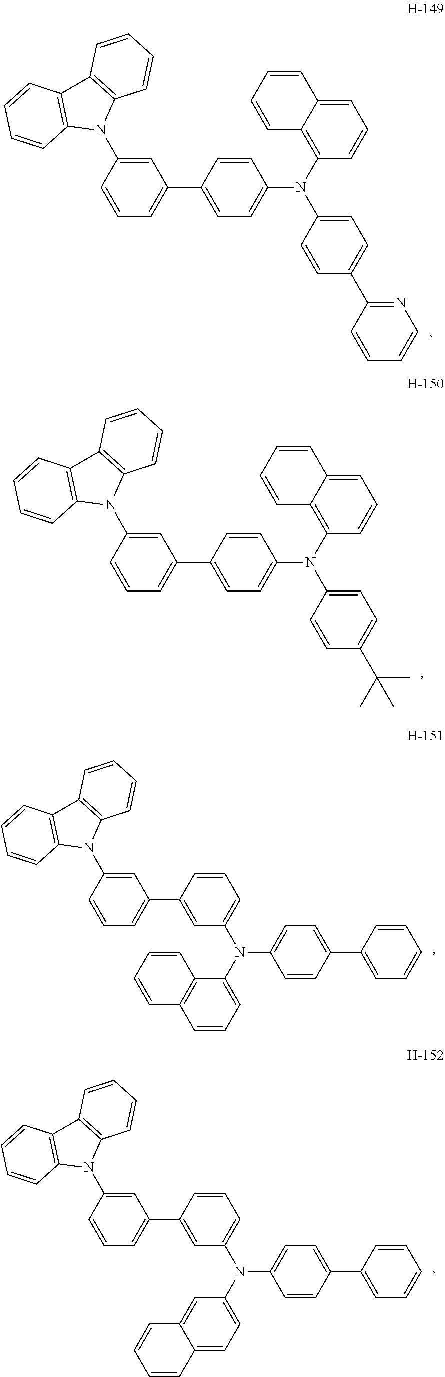 [SMILES: c1ccc(-c2ccc(N(c3ccc(-c4cccc(-n5c6ccccc6c6ccccc65)c4)cc3)c3cccc4ccccc34)cc2)nc1 ; CC(C)(C)c1ccc(N(c2ccc(-c3cccc(-n4c5ccccc5c5ccccc54)c3)cc2)c2cccc3ccccc23)cc1 ; c1ccc(-c2ccc(N(c3cccc(-c4cccc(-n5c6ccccc6c6ccccc65)c4)c3)c3cccc4ccccc34)cc2)cc1 ; c1ccc(-c2ccc(N(c3cccc(-c4cccc(-n5c6ccccc6c6ccccc65)c4)c3)c3ccc4ccccc4c3)cc2)cc1]

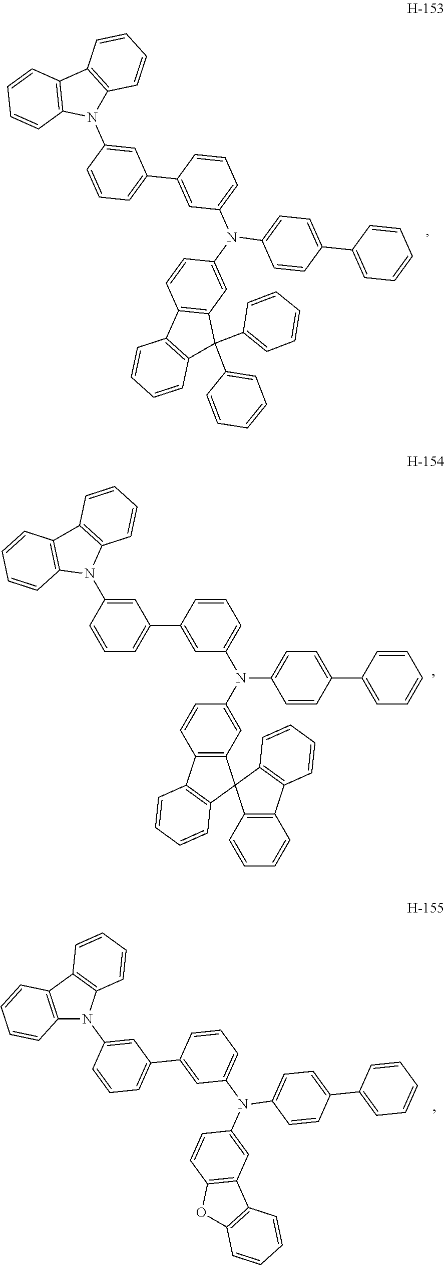 [SMILES: c1ccc(-c2ccc(N(c3cccc(-c4cccc(-n5c6ccccc6c6ccccc65)c4)c3)c3ccc4c(c3)C(c3ccccc3)(c3ccccc3)c3ccccc3-4)cc2)cc1 ; c1ccc(-c2ccc(N(c3cccc(-c4cccc(-n5c6ccccc6c6ccccc65)c4)c3)c3ccc4c(c3)C3(c5ccccc5-c5ccccc53)c3ccccc3-4)cc2)cc1 ; c1ccc(-c2ccc(N(c3cccc(-c4cccc(-n5c6ccccc6c6ccccc65)c4)c3)c3ccc4oc5ccccc5c4c3)cc2)cc1]

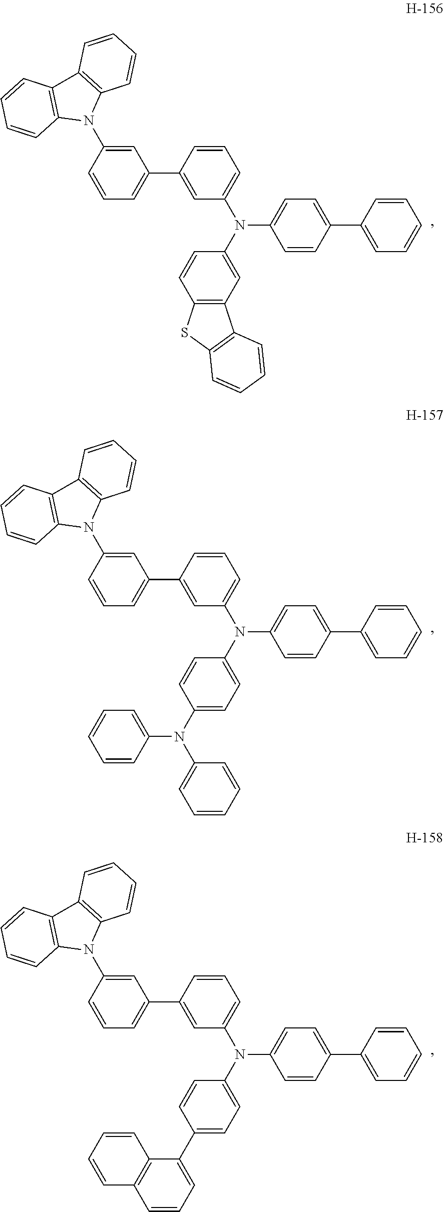 [SMILES: c1ccc(-c2ccc(N(c3cccc(-c4cccc(-n5c6ccccc6c6ccccc65)c4)c3)c3ccc4sc5ccccc5c4c3)cc2)cc1 ; c1ccc(-c2ccc(N(c3ccc(N(c4ccccc4)c4ccccc4)cc3)c3cccc(-c4cccc(-n5c6ccccc6c6ccccc65)c4)c3)cc2)cc1 ; c1ccc(-c2ccc(N(c3ccc(-c4cccc5ccccc45)cc3)c3cccc(-c4cccc(-n5c6ccccc6c6ccccc65)c4)c3)cc2)cc1]

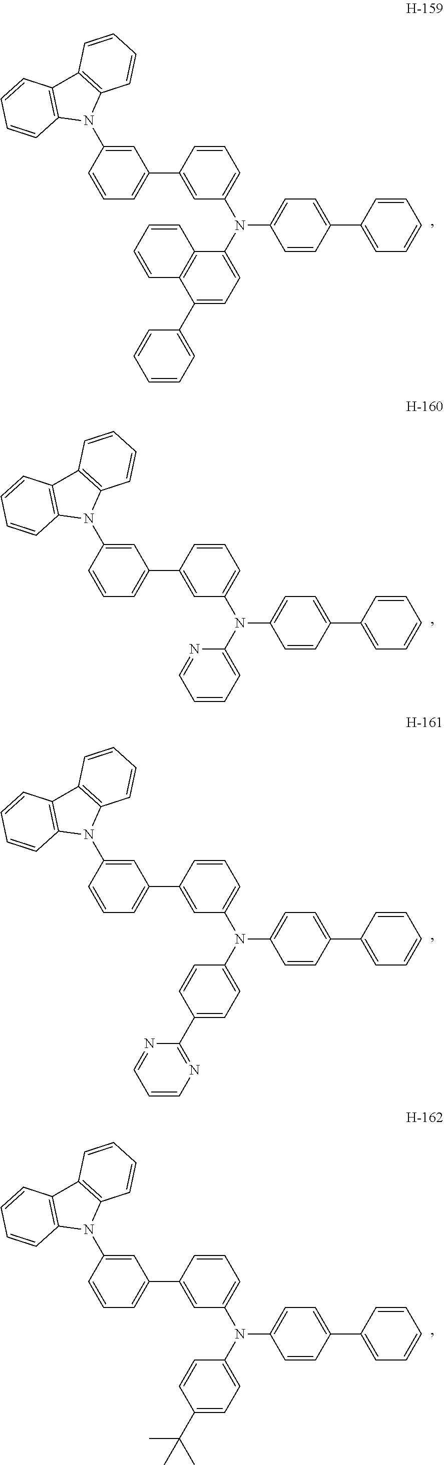 [SMILES: c1ccc(-c2ccc(N(c3cccc(-c4cccc(-n5c6ccccc6c6ccccc65)c4)c3)c3ccc(-c4ccccc4)c4ccccc34)cc2)cc1 ; c1ccc(-c2ccc(N(c3cccc(-c4cccc(-n5c6ccccc6c6ccccc65)c4)c3)c3ccccn3)cc2)cc1 ; c1ccc(-c2ccc(N(c3ccc(-c4ncccn4)cc3)c3cccc(-c4cccc(-n5c6ccccc6c6ccccc65)c4)c3)cc2)cc1 ; CC(C)(C)c1ccc(N(c2ccc(-c3ccccc3)cc2)c2cccc(-c3cccc(-n4c5ccccc5c5ccccc54)c3)c2)cc1]

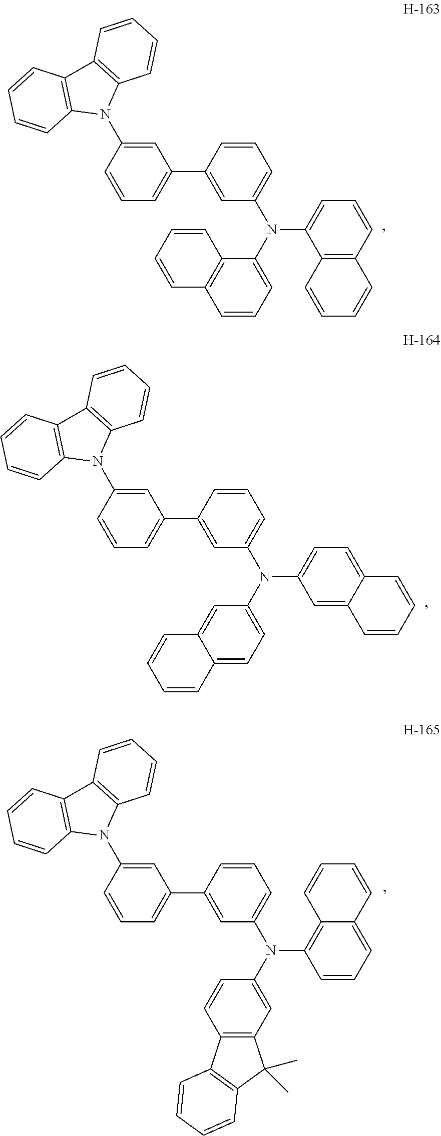 [SMILES: c1cc(-c2cccc(-n3c4ccccc4c4ccccc43)c2)cc(N(c2cccc3ccccc23)c2cccc3ccccc23)c1 ; c1cc(-c2cccc(-n3c4ccccc4c4ccccc43)c2)cc(N(c2ccc3ccccc3c2)c2ccc3ccccc3c2)c1 ; CC1(C)c2ccccc2-c2ccc(N(c3cccc(-c4cccc(-n5c6ccccc6c6ccccc65)c4)c3)c3cccc4ccccc34)cc21]

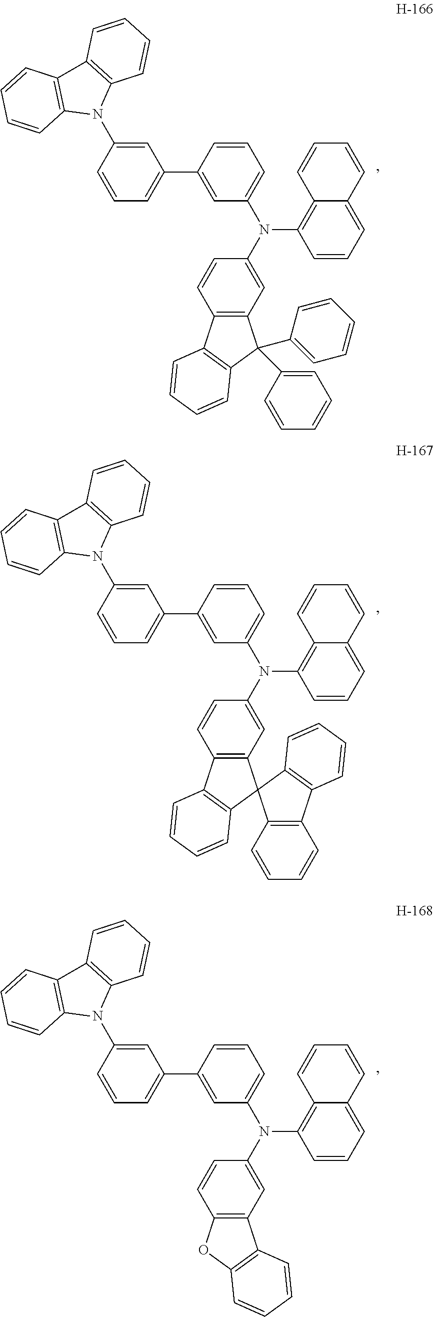 [SMILES: c1ccc(C2(c3ccccc3)c3ccccc3-c3ccc(N(c4cccc(-c5cccc(-n6c7ccccc7c7ccccc76)c5)c4)c4cccc5ccccc45)cc32)cc1 ; c1cc(-c2cccc(-n3c4ccccc4c4ccccc43)c2)cc(N(c2ccc3c(c2)C2(c4ccccc4-c4ccccc42)c2ccccc2-3)c2cccc3ccccc23)c1 ; c1cc(-c2cccc(-n3c4ccccc4c4ccccc43)c2)cc(N(c2ccc3oc4ccccc4c3c2)c2cccc3ccccc23)c1]

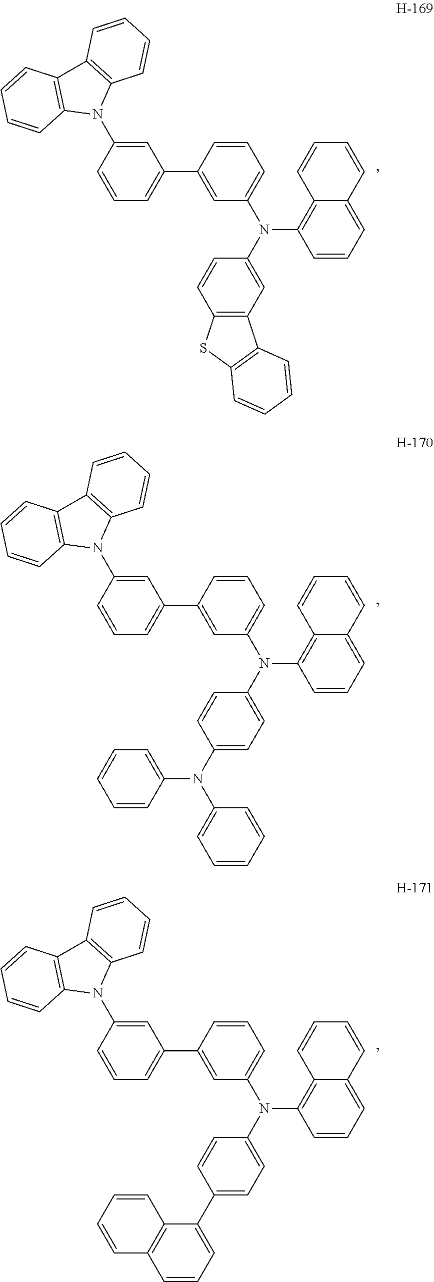 [SMILES: c1cc(-c2cccc(-n3c4ccccc4c4ccccc43)c2)cc(N(c2ccc3sc4ccccc4c3c2)c2cccc3ccccc23)c1 ; c1ccc(N(c2ccccc2)c2ccc(N(c3cccc(-c4cccc(-n5c6ccccc6c6ccccc65)c4)c3)c3cccc4ccccc34)cc2)cc1 ; c1cc(-c2cccc(-n3c4ccccc4c4ccccc43)c2)cc(N(c2ccc(-c3cccc4ccccc34)cc2)c2cccc3ccccc23)c1]

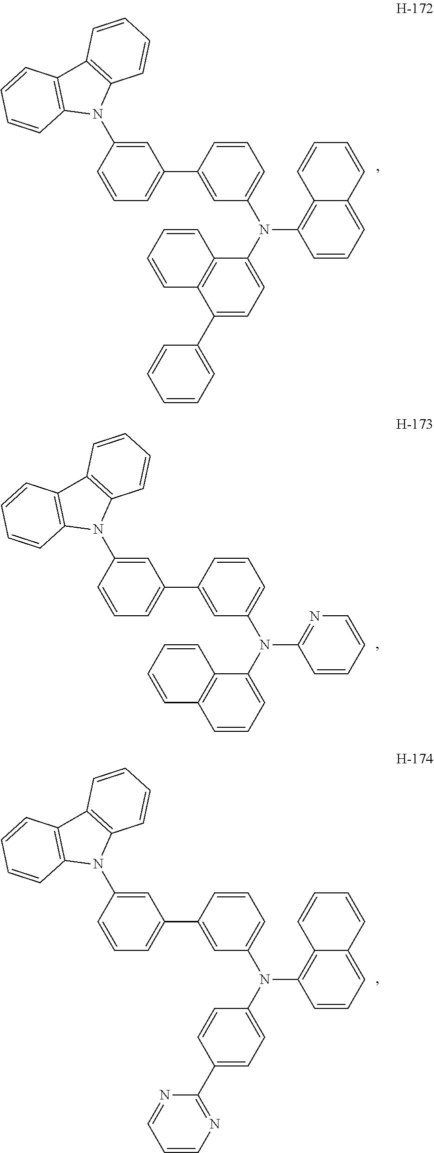 [SMILES: c1ccc(-c2ccc(N(c3cccc(-c4cccc(-n5c6ccccc6c6ccccc65)c4)c3)c3cccc4ccccc34)c3ccccc23)cc1 ; c1ccc(N(c2cccc(-c3cccc(-n4c5ccccc5c5ccccc54)c3)c2)c2cccc3ccccc23)nc1 ; c1cnc(-c2ccc(N(c3cccc(-c4cccc(-n5c6ccccc6c6ccccc65)c4)c3)c3cccc4ccccc34)cc2)nc1]

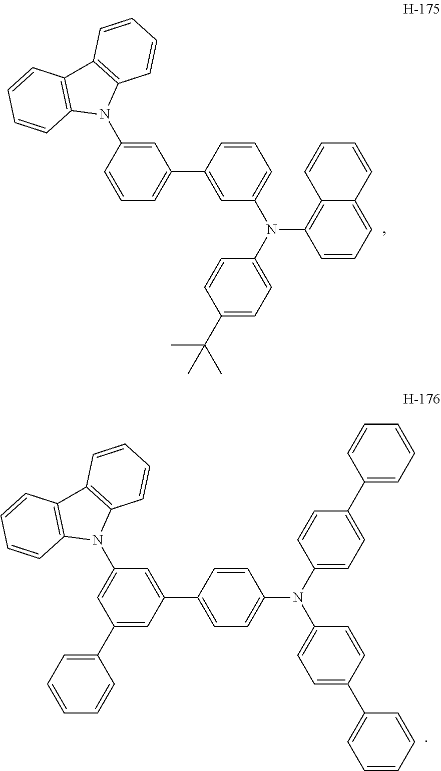 [SMILES: CC(C)(C)c1ccc(N(c2cccc(-c3cccc(-n4c5ccccc5c5ccccc54)c3)c2)c2cccc3ccccc23)cc1 ; c1ccc(-c2ccc(N(c3ccc(-c4ccccc4)cc3)c3ccc(-c4cc(-c5ccccc5)cc(-n5c6ccccc6c6ccccc65)c4)cc3)cc2)cc1]

According to an embodiment of the present disclosure, a display assembly is further disclosed. The display assembly includes the organic electroluminescent device according to any one of the preceding embodiments.

According to another embodiment of the present disclosure, a first organic electroluminescent device is disclosed. The first organic electroluminescent device comprises: a substrate;

a first electrode disposed on the substrate;

a second electrode disposed over the first electrode; and

an organic layer disposed between the first electrode and the second electrode;

wherein the first electrode is a material with high reflectivity or a combination of materials with high reflectivity, and the second electrode is a translucent or transparent material or a combination of translucent or transparent materials;

the organic layer comprises a first organic layer, a second organic layer and a third organic layer;

the first organic layer comprises a first organic material and a second organic material;

the second organic layer is made of the second organic material and has a first thickness;

the third organic layer is a light-emitting layer comprising at least one light-emitting material and at least one host material;

the first organic layer has a conductivity of greater than 1×10⁻⁴ S/m and less than 1×10⁻² S/m;

an energy level difference between a HOMO energy level of the second organic material and a HOMO energy level of the at least one host material is less than 0.27 eV;

a voltage of the first organic electroluminescent device is not higher than 110% of a voltage of a second organic electroluminescent device at the same current density, wherein the second organic electroluminescent device has the same device structure as the first organic electroluminescent device except the following differences:

(1) the first organic layer comprises the first organic material and a third organic material, wherein the third organic material is different from the second organic material;

(2) the second organic layer is made of the third organic material;

(3) a fourth organic layer is comprised between the second organic layer and the third organic layer, wherein the fourth organic layer is made of the second organic material;

wherein a total thickness of the second organic layer and the fourth organic layer in the second organic electroluminescent device is 90% to 110% of the first thickness in the first organic electroluminescent device.

According to an embodiment of the present disclosure, the voltage of the first organic electroluminescent device is not higher than the voltage of the second organic electroluminescent device at the same current density.

According to an embodiment of the present disclosure, the HOMO energy level of the second organic material in the first organic electroluminescent device is less than a HOMO energy level of the third organic material in the second organic electroluminescent device.

According to an embodiment of the present disclosure, the HOMO energy level of the second organic material in the first organic electroluminescent device is less than −5.25 eV.

According to an embodiment of the present disclosure, a LUMO energy level of the first organic material in the first organic electroluminescent device is less than −5.1 eV.

According to an embodiment of the present disclosure, an energy level difference between the HOMO energy level of the second organic material and the LUMO energy level of the first organic material is less than 0.23 eV.

According to an embodiment of the present disclosure, the energy level difference between the HOMO energy level of the second organic material and the LUMO energy level of the first organic material is less than 0.2 eV.

According to an embodiment of the present disclosure, the energy level difference between the HOMO energy level of the second organic material and the LUMO energy level of the first organic material is less than or equal to 0.1 eV.

According to an embodiment of the present disclosure, the second organic layer in the first organic electroluminescent device has a thickness of greater than 80 nm.

According to an embodiment of the present disclosure, the second organic layer in the first organic electroluminescent device has a thickness of greater than 125 nm.

According to an embodiment of the present disclosure, the second organic layer in the first organic electroluminescent device has a thickness of greater than or equal to 100 nm.

According to an embodiment of the present disclosure, the second organic layer in the first organic electroluminescent device has a thickness of greater than or equal to 120 nm.

According to an embodiment of the present disclosure, the second organic layer in the first organic electroluminescent device has a thickness of greater than 150 nm.

According to an embodiment of the present disclosure, a display assembly is further disclosed. The display assembly includes the first organic electroluminescent device according to any one of the preceding embodiments.

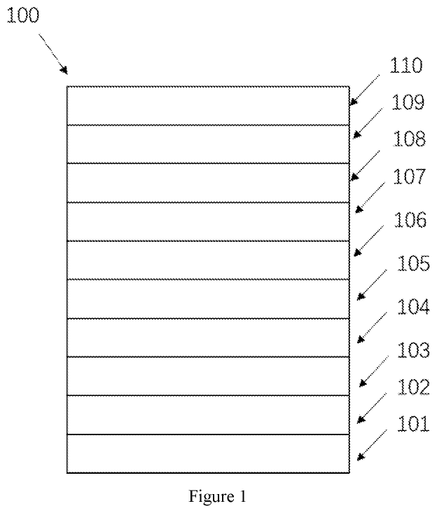

The structural diagram of a typical top-emitting OLED device is shown in FIG. 1. An OLED device 100 includes an anode layer 101, a hole injection layer (HIL) 102, a hole transporting layer (HTL) 103, an electron blocking layer (EBL) 104, an emissive layer (EML) 105, a hole blocking layer (HBL) 106, an electron transporting layer (ETL) 107, an electron injection layer (EIL) 108, a cathode layer 109 and a capping layer 110. The anode layer 101 is a material with high reflectivity or a combination of materials with high reflectivity, where the material includes, but is not limited to, Ag, Ti, Cr, Pt, Ni, TiN and combinations thereof with ITO and/or MoOx (molybdenum oxide) and the material generally has a reflectivity of greater than 50%, preferably, greater than 80%, and more preferably, greater than 90%. The cathode layer 109 should be a translucent or transparent conductive material, where the material includes, but is not limited to, a Mg—Ag alloy, MoOx, Yb, Ca, ITO, IZO or a combination thereof and the material generally has a transparency of greater than 30%, preferably, greater than 50%. The electron transporting layer 107 may be a single layer of Yb. The emissive layer 105 generally includes at least one host material and at least one light-emitting material, and the hole blocking layer 106 is an optional layer. To ensure that excitons are not quenched at an interface between the EBL and the EML, it is generally necessary to ensure that a material of the EBL has a higher triplet energy level than the host material in the EML. The hole injection layer 102 may be a single material layer such as commonly used HATCN. The hole injection layer 102 may also be a hole transporting material doped with a certain proportion of conductive p-type doping material, where the doping proportion is generally not higher than 5% and commonly between 1% and 3%. The hole injection layer doped with a conductive p-type material generally has a lower voltage than the single material layer and thus is widely applied. A commonly used material of the hole transporting layer, such as Compound HT1 in Table 1, has a HOMO energy level of −5.09 eV, which is close to a work function of −4.8 eV of commonly used ITO for the anode layer, ensuring the effective injection of holes from the anode layer. However, most host materials of the emissive layer generally have HOMO energy levels of −5.3 eV to −5.6 eV (such as Compound RH1 and RH1 in Table 1), which are much deeper than that of the material of the hole transporting layer so that holes face a relatively high potential barrier when they enter the emissive layer from the transporting layer. If the HOMO energy level of a hole transporting material can be close to that of the host material, the potential barrier before the holes are transported into the emissive layer will be reduced or even disappear. However, too deep a HOMO energy level makes it difficult to inject holes from the anode layer and results in worse ohmic contact, causing an increase in voltage. It has been found through researches that this phenomenon can be alleviated by doping the conductive p-type doping material into the material of the hole injection layer with a deep energy level. However, a commonly used conductive p-type doping material, such as Compound HT in Table 2, has a LUMO energy level of only −5.04 eV. The inventor of the present application has found that such p-type doping material cannot form a good doping effect with the hole transporting material with a deep energy level. For better matching, the LUMO energy level of the conductive p-type doping material also needs to be deeper.

In the present disclosure, the electrochemical properties of all compounds are measured through cyclic voltammetry (CV). The test is conducted using an electrochemical workstation modelled CorrTest CS120 produced by Wuhan Corrtest Instruments Corp., Ltd and using a three-electrode working system where: a platinum disk electrode serves as a working electrode, a Ag/AgNO₃ electrode serves as a reference electrode, and a platinum wire electrode serves as an auxiliary electrode. Anhydrous DCM is used as a solvent, 0.1 mol/L tetrabutylammonium hexafluorophosphate is used as a supporting electrolyte, a compound to be tested is prepared into a solution of 10⁻³ mol/L, and nitrogen is introduced into the solution for 10 min for oxygen removal before the test. The parameters of the instrument are set as follows: a scan rate of 100 mV/s, a potential interval of 0.5 mV and a test window of −1 V to 1 V. The HOMO energy levels of some hole transporting materials (HTMs) and some host materials measured by the above test method are listed in Table 1, and the LUMO energy levels of some PD materials measured by the above test method are listed in Table 2.

TABLE 1
HOMO energy levels of some hole transporting materials, host
materials for red light and a host material for blue light

| Material | Function | HOMO (eV) |
|---|---|---|
| Compound HT1 | Hole transport | −5.09 |
| Compound H-176 | Hole transport | −5.27 |
| Compound RH1 | Host material for red-emitting dopant | −5.39 |
| Compound RH2 | Host material for red-emitting dopant | −5.36 |
| Compound BH | Host material for blue-emitting dopant | −5.53 |

TABLE 2
LUMO energy levels of some PD materials

| Material | LUMO (eV) |
|---|---|
| Compound HT | −5.04 |
| Compound 70 | −5.17 |
| Compound 72 | −5.17 |
| Compound 56 | −5.11 |

Compound HT1, H-176, Compound 70, Compound 72, Compound 56, Compound HT, Compound RH1, Compound RH2 and Compound BH have the following structural formulas:

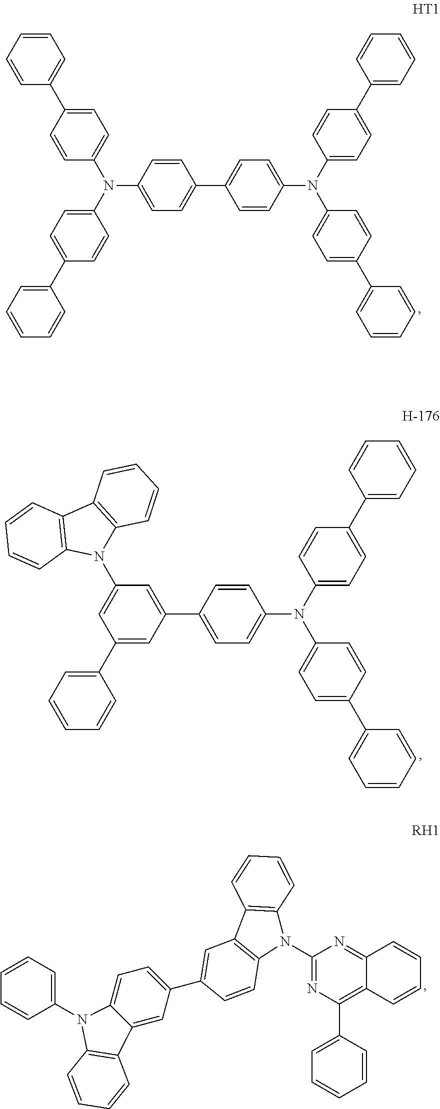 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(N(c3ccc(-c4ccccc4)cc3)c3ccc(-c4cc(-c5ccccc5)cc(-n5c6ccccc6c6ccccc65)c4)cc3)cc2)cc1 ; c1ccc(-c2nc(-n3c4ccccc4c4cc(-c5ccc6c(c5)c5ccccc5n6-c5ccccc5)ccc43)nc3ccccc23)cc1]

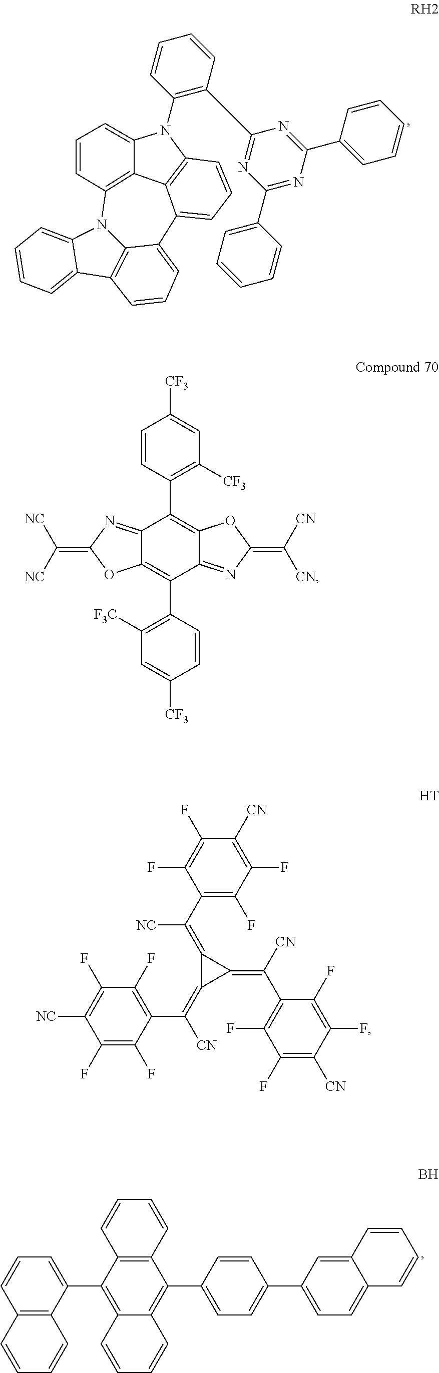

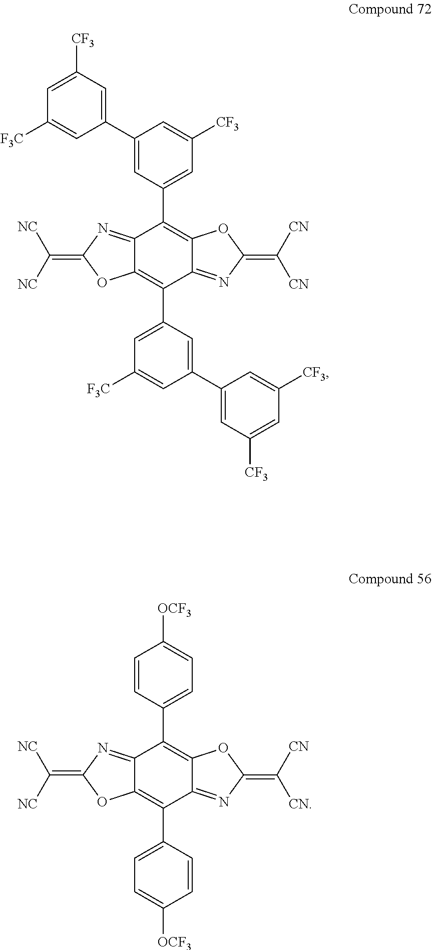 [SMILES: N#CC(C#N)=c1nc2c(-c3cc(-c4cc(C(F)(F)F)cc(C(F)(F)F)c4)cc(C(F)(F)F)c3)c3oc(=C(C#N)C#N)nc3c(-c3cc(-c4cc(C(F)(F)F)cc(C(F)(F)F)c4)cc(C(F)(F)F)c3)c2o1 ; N#CC(C#N)=c1nc2c(-c3ccc(OC(F)(F)F)cc3)c3oc(=C(C#N)C#N)nc3c(-c3ccc(OC(F)(F)F)cc3)c2o1]

Though matching the energy levels of the HTM and the PD material is the first step to ensure effective hole injection, the doping ratio of the PD material also affects the hole injection ability. The hole injection ability of the hole injection layer can be quantitatively analyzed by measuring the conductivity of the hole injection layer. Generally, within a certain range, the higher the doping ratio of the PD material, the higher the conductivity, that is, the stronger the hole injection ability. If the conductivity is too low, insufficient hole injection will lead to an increase in voltage, and the recombination region in the EML will move towards the anode, which may also lead to a decrease in lifetime. On the contrary, if the conductivity is too high, excessive hole injection will lead to a decrease in efficiency, which is obvious especially in an electron-deficient system. Moreover, in display applications, too high a conductivity of the HIL will also bring about the problem of lateral crosstalk between pixels. Therefore, the conductivity of the HIL should be within a certain range, for example, 1×10⁻⁴ to 1×10⁻² S/m, preferably, 2×10⁻⁴ to 8×10⁻³ S/m.

The conductivity is measured by the following method: the to-be-tested samples of the HTM and the PD material are co-deposited through evaporation on a test substrate pre-prepared with an aluminum electrode at a certain doping ratio (the PD material in Table 2 is doped with the HTM in Table 1 at a weight ratio of 3%, 2% and 1%) at a vacuum degree of 10⁻⁶ torr to form a to-be-tested region with a thickness of 100 nm, a length of 6 mm and a width of 1 mm, a voltage is applied to the electrode and a current is measured to obtain a resistance value of the region, and then the conductivity of the film layer is calculated according to the Ohm's law and geometric dimensions. It is to be noted that even if the HTM and the PD material are kept unchanged, that is, their energy level difference remains unchanged, the hole injection capability can be adjusted to a certain extent by adjusting the doping ratio. On the other hand, if the difference between the energy levels of the HTM and the PD material is too large, the hole injection ability is adjusted by the doping ratio to a very limited extent. The measurement results of the conductivities of some HTMs with different proportions of PD measured by the above conductivity test method are listed in Table 3.

TABLE 3
Measurement results of the conductivities of some HTMs with different PDs

| Material | Conductivity | Material | Conductivity | Material | Conductivity |
|---|---|---|---|---|---|
| Combination | (10⁻⁴ S/m) | Combination | (10⁻⁴ S/m) | Combination | (10⁻⁴ S/m) |
| HT1:HT (3%) | 69.7 | HT1:HT (2%) | 32.0 | HT1:HT (1%) | 10.0 |
| HT1:Compound | 70.1 | HT1:Compound | 40.4 | HT1:Compound | 17.7 |
| 70 (3%) | | 70 (2%) | | 70 (1%) | |
| H-176:HT (3%) | 1.2 | H-176:HT (2%) | 1.0 | H-176:HT (1%) | 0.3 |
| H-176:Compound | 6.2 | H-176:Compound | 4.6 | H-176:Compound | 3.2 |
| 70 (3%) | | 70 (2%) | | 70 (1%) | |

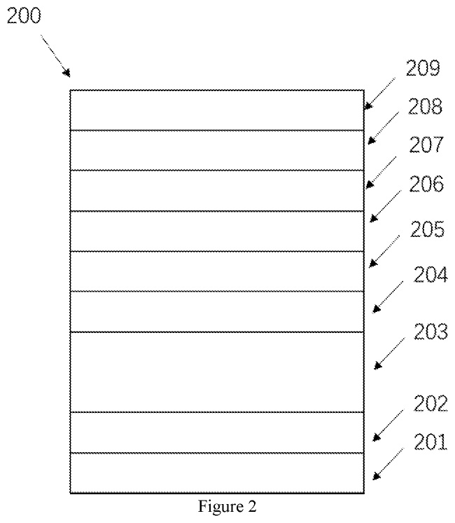

FIG. 2 is a structural diagram of a simplified top-emitting device. An OLED device 200 includes an anode layer 201, a hole injection layer (HIL) 202, a hole transporting layer (HTL) 203, an emissive layer (EML) 204, a hole blocking layer (HBL) 205, an electron transporting layer (ETL) 206, an electron injection layer (EIL) 207, a cathode layer 208 and a capping layer 209. Similarly, the emissive layer 204 generally includes at least one host material and at least one light-emitting material, and the hole blocking layer 205 is an optional layer. The thickness of the hole transporting layer 203 should be comparable to a sum of thicknesses of all film layers between the HIL and the EML in a conventional top-emitting device and can be fine-tuned according to a microcavity effect. The thickness of the hole transporting layer 203 is generally greater than 80 nm, preferably, greater than 125 nm, and more preferably, greater than 150 nm. In the preceding structure of the simplified top-emitting device, since the thickness of the HTL increases, the amount of holes reaching the emissive layer decreases and the recombination region will move towards the anode. In the structure of the simplified top-emitting device, since there is no EBL, it is necessary to ensure that the HTM in the HTL in direct contact with the EML has a higher triplet energy level than the host material in the EML to ensure that excitons are not quenched at an interface between the HTL and the EML. In the simplified top-emitting OLED device 200 shown in FIG. 2, the hole transporting material (HTM) used in the hole transporting layer 203 has a deep HOMO energy level, and the difference between the HOMO energy level of the HTM and a HOMO energy level of at least one host material in the emissive layer 204 is less than 0.27 eV, preferably, less than 0.25 eV, and more preferably, less than 0.2 eV. The relatively small energy level difference reduces the potential barrier for holes entering the EML, which can effectively reduce the voltage and offset the voltage increase due to too thick the HTM especially in the top-emitting device. With reference to the HOMO energy levels of the materials in Table 1, the differences between the HOMO energy levels of the HTMs and the HOMO energy level of the host materials in Table 1 and the differences between the HOMO energy levels of the HTMs and the LUMO energy levels of the PD materials in Table 2, that is, HOMO_{HTM}-HOMO_{RH} and LUMO_{PD}-HOMO_{HTM}, are listed in Table 4. It can be seen that the energy level differences between the hole transporting material Compound H-176 and the host materials RH1 and RH2 are 0.12 eV and 0.09 eV, respectively, which are both less than 0.27 eV. To ensure good hole injection, the energy levels of the HTM and the PD material are also to be matched, that is, (LUMO_{PD}-HOMO_{HTM}) is less than 0.23 eV, preferably, less than 0.2 eV, and more preferably, less than 0.1 eV. In particular, the HTM with a deep HOMO energy level such as Compound H-176, when matched with the PD material with a deep LUMO energy level such as Compound 70, can achieve more effective hole injection. As shown in Table 4, their energy level difference is 0.1 eV.

TABLE 4
Differences between the HOMO energy levels of some HTMs and the HOMO energy level
of some host materials for red light and blue light and differences between the
HOMO energy levels of the HTMs and the LUMO energy levels of some PD materials

| | Material | | | | |
|---|---|---|---|---|---|
| | Compound | Compound | Compound | Compound | Compound |
| Energy Level Difference [eV] | RH1 | RH2 | BH | HT | 70 |
| Compound HT1 | 0.30 | 0.27 | 0.44 | 0.05 | −0.08 |
| Compound H-176 | 0.12 | 0.09 | 0.26 | 0.23 | 0.1 |

### Device Example

Hereinafter, the present disclosure is described in more detail with reference to the following examples. The compounds used in the following examples can be easily obtained by those skilled in the art, so synthesis methods of these compounds will not be repeated here. For example, the synthesis methods are available from the Chinese patent application CN112745333A, which is incorporated by reference in its entirety. Apparently, the following examples are only for the purpose of illustration and not intended to limit the scope of the present disclosure. Based on the following examples, those skilled in the art can obtain other examples of the present disclosure by conducting improvements on these examples.

### Example 1: Preparation of an Organic Electroluminescent Device 200 Shown in FIG. 2

Firstly, a 0.7 mm thick glass substrate was pre-patterned with indium tin oxide (ITO) 75 Å/Ag 1500 Å/ITO 150 Å for use as an anode 201, where 150 Å ITO deposited on Ag had a hole injection function. Then, the substrate was dried in a glovebox to remove moisture, mounted on a holder and transferred into a vacuum chamber. Organic layers specified below were sequentially deposited through vacuum thermal evaporation on the anode layer at a rate of 0.01-10 Å/s and at a vacuum degree of about 10⁻⁶ torr. Compound H-176 and Compound 70 (98:2, 100 Å) were co-deposited for use as a hole injection layer (HIL) 202. Compound H-176 (1900 Å) was deposited for use as a hole transporting layer (HTL) 203 and a microcavity length adjustment layer. Compound RH1 and Compound RD (98:2, 400 Å) were co-deposited on the HTL for use as an emissive layer (EML) 204. Compound HB (50 Å) was deposited for use as a hole blocking layer (HBL) 205. Compound ET and Liq (40:60, 350 Å) were co-deposited for use as an electron transporting layer (ETL) 206. A metal Yb (10 Å) was deposited for use as an electron injection layer (EIL) 207. Metals Ag and Mg (9:1, 140 Å) were co-deposited for use as a cathode 208. Finally, Material CPL (650 Å) was deposited for use as a capping layer 209 (the CPL material has a refractive index of about 1.68 at 620 nm, and the refractive index is obtained by testing a 30 nm thick CPL material deposited on a silicon wafer using an ES01 ellipsometer from BEIJING ELLITOP). The device was transferred back to the glovebox and encapsulated with a glass lid to complete the device.

### Comparative Example 1-1: Preparation of an Organic Electroluminescent Device 100 Shown in FIG. 1

This comparative example adopted the same preparation method as Example 1, except that Compound HT1 and Compound 70 (98:2, 100 Å) were co-deposited for use as a hole injection layer (HIL) 102, Compound HT1 (1200 Å) was deposited for use as a hole transporting layer (HTL) 103, and Compound H-176 (700 Å) was deposited for use as an electron blocking layer (EBL) 104 and a microcavity length adjustment layer.

### Comparative Example 1-2: Preparation of an Organic Electroluminescent Device 200 Shown in FIG. 2

This comparative example adopted the same preparation method as Example 1, except that Compound H-176 and Compound HT (98:2, 100 Å) were co-deposited for use as a hole injection layer (HIL) 202.

Detailed structures and thicknesses of part of layers of the devices are shown in Table 5. A layer using more than one material is obtained by doping different compounds at their weight ratio as recorded.

TABLE 5
Device structures of some organic layers in Example
1 and Comparative Examples 1-1 and 1-2

| No. | HIL | HTL | EBL | EML |
|---|---|---|---|---|
| Example 1 | H-176:70 | H-176 | / | RH1:RD |
| | (98:2) (100 Å) | (1900 Å) | | (98:2) (400 Å) |
| Comparative | HT1:70 | HT1 | H-176 | RH1:RD |
| Example 1-1 | (98:2) (100 Å) | (1200 Å) | (700 Å) | (98:2) (400 Å) |
| Comparative | H-176:HT | H-176 | / | RH1:RD |
| Example 1-2 | (98:2) (100 Å) | (1900 Å) | | (98:2) (400 Å) |

Compounds RD, HB, ET and Liq have the following structural formulas:

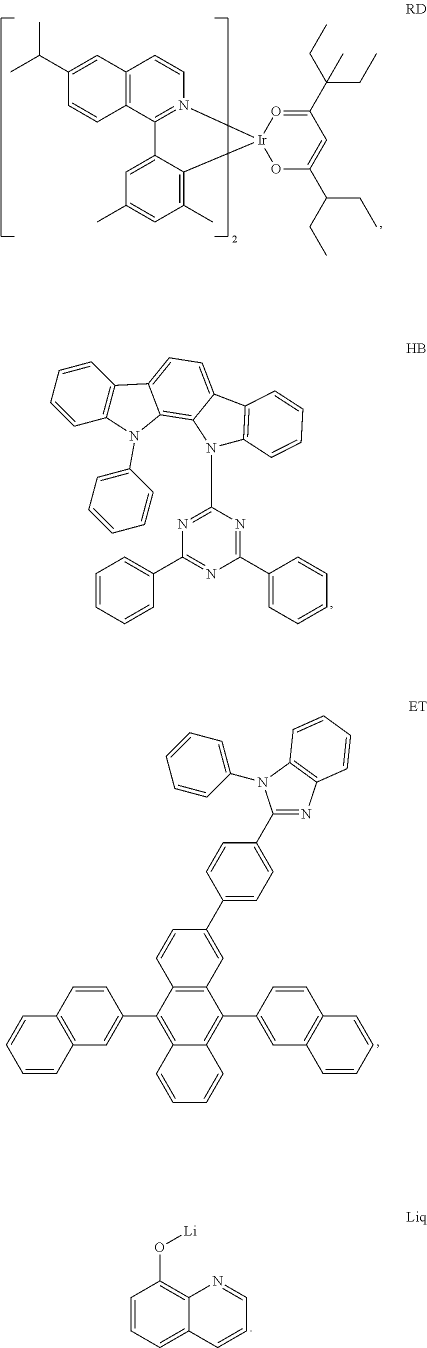

The device performance of Example 1 and Comparative Examples 1-1 and 1-2 is shown in Table 6. The color coordinates, voltage and current efficiency were measured at a current density of 10 mA/cm², and the device lifetime (LT97) was the measured time taken for the device to decay to 97% of its initial brightness at 80 mA/cm².

TABLE 6
Device performance of Example 1 and Comparative Examples 1-1 and 1-2

| | | | | HOMO | | | | | |
|---|---|---|---|---|---|---|---|---|---|
| | | HOMO | LUMO | Energy | At 10 mA/cm² | | | | |
| | Conductivity | Energy | Energy | Level | | | | Current | At 80 |
| | of the | Level | Level | of the | | | | Efficiency | mA/cm² |
| | HIL/10⁻⁴ | of the | of the | Host | | | Voltage | (CE) | LT97 |
| | S/m | HTM/eV | PD/eV | Material/eV | CIEx | CIEy | [V] | [cd/A] | [h] |
| Example 1 | 4.6 | −5.27 | −5.17 | −5.39 | 0.683 | 0.316 | 5.1 | 62 | 105 |
| Comparative | 40.4 | −5.09 | −5.17 | −5.39 | 0.682 | 0.318 | 5.5 | 60 | 101 |
| Example 1-1 | | | | | | | | | |
| Comparative | 1.0 | −5.27 | −5.04 | −5.39 | 0.682 | 0.318 | 8.5 | 63 | 58 |
| Example 1-2 | | | | | | | | | |

The device in Example 1 uses Compound 70 with a LUMO energy level of −5.17 eV as the conductive p-type doping material which is doped into Compound H-176 with a HOMO energy level of −5.27 eV for use as the material of the hole injection layer. It can be seen from Table 4 that the energy level difference between the HOMO energy level of the HTM and the LUMO energy level of the PD is 0.1 eV. It can be seen from Table 3 that at a doping proportion of 2%, the conductivity of the hole injection layer is 4.6×10⁻⁴ S/m, which is greater than 1×10⁻⁴ S/m, indicating good hole injection from the anode to the organic layer. It is to be noted that it can be seen from Table 3 that if the doping proportion of Compound 70 is reduced to, for example, 1%, the conductivity can be reduced; on the contrary, if the doping proportion of Compound 70 is increased to 3%, the conductivity can be improved. Comparative Example 1-1 is a red light device structure commonly used in the industry, and it can be seen from the device data that the device has relatively high red light device performance in the industry. Compared with Comparative Example 1-1, Example 1 has slightly improved efficiency, a slightly prolonged lifetime and a voltage reduced by 0.4 V on the premise of ensuring its color. As can be seen from Table 3, the HIL used in Comparative Example 1-1 has a conductivity of 40.4×10⁻⁴ S/m and has better hole injection than that in Example 1. However, Comparative Example 1-1 has a higher voltage than Example 1. As can be seen from Table 4, the energy level difference between the HOMO energy level of the HTM (H-176) in the HIL in Example 1 and the HOMO energy level of the host material RH1 for red light is 0.12 eV, while the energy level difference between the HOMO energy level of the HTM (HT1) in the HIL in Comparative Example 1-1 and the HOMO energy level of RH1 is 0.30 eV. This indicates that a decrease of the energy level difference between the HOMO energy levels of the HTM and the host material in the emissive layer has a decisive effect on the voltage of the device; secondly, a decrease of the number of function layers can also reduce the number of defects caused by an interface, which is also helpful for reducing the voltage.

The hole injection layer in Comparative Example 1-2 uses Compound HT for p-doping and H-176 as the HTM. It can be seen from Table 3 that the conductivity of the hole injection layer is 1×10⁻⁴ S/m, which is lower than that in Example 1 so that it can be seen that the hole injection layer has a worse hole injection ability than the HIL in Example 1. Similarly, the hole injection ability can be embodied by the energy level difference. Comparative Example 1-2 uses Compound HT with a LUMO energy level of −5.04 eV as the conductive p-type doping material which is doped into Compound H-176 with a HOMO energy level of −5.27 eV for use as the material of the hole injection layer. It can be seen from Table 4 that the energy level difference between the HOMO energy level of the HTM and the LUMO energy level of the PD is 0.23 eV, which is higher than 0.1 eV in Example 1 so that the hole injection layer has a worse hole injection ability than the HIL in Example 1 at the same doping ratio. Therefore, Comparative Example 1-2 has a voltage as high as 8.5 V and a lifetime reduced by 45% though it can maintain basically the same current efficiency as Example 1. Here, the energy level difference between the HOMO energy levels of the HTM (H-176) in Comparative Example 1-2 and the host material RH1 for red light is 0.12 eV, which is the same as that in Example 1, and the difference only lies in that under the same doping concentration, the hole injection layers have different conductivities.

As can be seen from the comparison of the above example and comparative examples, the energy level difference between the HOMO energy levels of the HTM and the host material in the emissive layer and the conductivity of the hole injection layer both have important effects on the device performance, especially the voltage and lifetime of the device. Example 1 which satisfies both the conductivity and the energy level difference in the present application can further reduce the device voltage and prolong the device lifetime when the CIE and the efficiency are basically unchanged.

### Example 2: Preparation of an Organic Electroluminescent Device 200 Shown in FIG. 2

This example adopted the same preparation method as Example 1, except that Compound RH2 and Compound RD (98:2, 400 Å) were co-deposited for use as an emissive layer (EML) 204.

### Comparative Example 2-1: Preparation of an Organic Electroluminescent Device 100 Shown in FIG. 1

This comparative example adopted the same preparation method as Comparative Example 1-1, except that Compound RH2 and Compound RD (98:2, 400 Å) were co-deposited for use as an emissive layer (EML) 105.

### Comparative Example 2-2: Preparation of an Organic Electroluminescent Device 200 Shown in FIG. 2

This comparative example adopted the same preparation method as Example 2, except that Compound H-176 and Compound HT (98:2, 100 Å) were co-deposited for use as a hole injection layer (HIL) 202.

Detailed structures and thicknesses of part of layers of the devices are shown in Table 7. A layer using more than one material is obtained by doping different compounds at their weight ratio as recorded.

TABLE 7
Device structures of some organic layers in Example
2 and Comparative Examples 2-1 and 2-2

| No. | HIL | HTL | EBL | EML |
|---|---|---|---|---|
| Example 2 | H-176:70 | H-176 | / | RH2:RD |
| | (98:2) (100 Å) | (1900 Å) | | (98:2) (400 Å) |
| Comparative | HT1:70 | HT1 | H-176 | RH2:RD |
| Example 2-1 | (98:2) (100 Å) | (1200 Å) | (700 Å) | (98:2) (400 Å) |
| Comparative | H-176:HT | H-176 | / | RH2:RD |
| Example 2-2 | (98:2) (100 Å) | (1900 Å) | | (98:2) (400 Å) |

The device performance of Example 2 and Comparative Examples 2-1 and 2-2 is shown in Table 8. The color coordinates, voltage and current efficiency were measured at a current density of 10 mA/cm², and the device lifetime (LT97) was the measured time taken for the device to decay to 97% of its initial brightness at 80 mA/cm².

TABLE 8
Device performance of Example 2 and Comparative Examples 2-1 and 2-2

| | | | | HOMO | | | | | |
|---|---|---|---|---|---|---|---|---|---|
| | | HOMO | LUMO | Energy | At 10 mA/cm² | | | | |
| | Conductivity | Energy | Energy | Level | | | | Current | At 80 |
| | of the | Level | Level | of the | | | | Efficiency | mA/cm² |
| | HIL/10⁻⁴ | of the | of the | Host | | | Voltage | (CE) | LT97 |
| | S/m | HTM/eV | PD/eV | Material/eV | CIEx | CIEy | [V] | [cd/A] | [h] |
| Example 2 | 4.6 | −5.27 | −5.17 | −5.36 | 0.670 | 0.330 | 4.1 | 47 | 165 |
| Comparative | 40.4 | −5.09 | −5.17 | −5.36 | 0.671 | 0.329 | 4.6 | 49 | 130 |
| Example 2-1 | | | | | | | | | |
| Comparative | 1.0 | −5.27 | −5.04 | −5.36 | 0.670 | 0.330 | 7.3 | 49 | 93 |
| Example 2-2 | | | | | | | | | |

The hole injection layer of the device in Example 2 is the same as that in Example 1 and has good hole injection from the anode to the organic layer. Comparative Example 2-1 is a red light device structure commonly used in the industry, and it can be seen from the device data that the device has relatively high red light device performance in the industry. Compared with Comparative Example 2-1, Example 2 has a voltage reduced by 0.5 V, a lifetime prolonged by 27% and comparable device efficiency on the premise of ensuring its color. This is because the energy level difference between the HOMO energy levels of the HTM (H-176) in Example 2 and the host material RH2 for red light has an absolute value of 0.09 eV, while the difference is 0.27 eV in Comparative Example 2-1. A smaller potential barrier results in a decrease in voltage and also ensures that holes can be effectively transported to the emissive layer.

Similar to that in Comparative Example 1-2, the hole injection layer in Comparative Example 2-2 uses Compound HT for p-doping and H-176 as the HTM. It can be seen from Table 3 that the conductivity of the hole injection layer is 1×10⁻⁴ S/m, which is lower than that in Example 2 so that it can be seen that the hole injection layer has a worse hole injection ability than the HIL in Example 2. Similarly, the hole injection ability can be embodied by the energy level difference. Comparative Example 2-2 uses Compound HT with a LUMO energy level of −5.04 eV as the conductive p-type doping material which is doped into Compound H-176 with a HOMO energy level of −5.27 eV for use as the material of the hole injection layer. It can be seen from Table 4 that the energy level difference between the HOMO energy level of the HTM and the LUMO energy level of the PD is 0.23 eV, which is higher than 0.1 eV in Example 2. Therefore, Comparative Example 2-2 has a voltage as high as 7.3 V and a lifetime reduced by 44% relative to the lifetime in Example 2 though it can maintain basically the same current efficiency as Example 2. Here, the energy level difference between the HOMO energy levels of the HTM (H-176) in Comparative Example 2-2 and the host material RH2 for red light has an absolute value of 0.09 eV, which is the same as that in Example 2, and the difference only lies in that the hole injection layers have different conductivities.

As can be seen from the comparison of the above example and comparative examples, the energy level difference between the HOMO energy levels of the HTM and the host material in the emissive layer and the conductivity of the hole injection layer both have important effects on the device performance, especially the voltage and lifetime of the device. Example 2 which satisfies both the conductivity and the energy level difference in the present application can further reduce the device voltage and prolong the device lifetime when the CIE and the efficiency are basically unchanged.

### Example 3-1: Preparation of an Organic Electroluminescent Device 200 Shown in FIG. 2

Firstly, a 0.7 mm thick glass substrate was pre-patterned with indium tin oxide (ITO) 75 Å/Ag 1500 Å/ITO 150 Å for use as an anode 201, where 150 Å ITO deposited on Ag had a hole injection function. Then, the substrate was dried in a glovebox to remove moisture, mounted on a holder and transferred into a vacuum chamber. Organic layers specified below were sequentially deposited through vacuum thermal evaporation on the anode layer at a rate of 0.01-10 Å/s and at a vacuum degree of about 10⁻⁶ torr. Compound H-176 and Compound 70 (98:2, 100 Å) were co-deposited for use as a hole injection layer (HIL) 202. Compound H-176 (1210 Å) was deposited for use as a hole transporting layer (HTL) 203 and a microcavity length adjustment layer. Compound BH and Compound BD (98:2, 200 Å) were co-deposited on the HTL for use as an emissive layer (EML) 204. Compound HB2 (50 Å) was deposited for use as a hole blocking layer (HBL) 205. Compound ET and Liq (40:60, 300 Å) were co-deposited for use as an electron transporting layer (ETL) 206. A metal Yb (10 Å) was deposited for use as an electron injection layer (EIL) 207. Metals Ag and Mg (9:1, 140 Å) were co-deposited for use as a cathode 208. Finally, Material CPL (650 Å) was deposited for use as a capping layer 209 (the CPL material has a refractive index of about 1.68 at 620 nm, and a 30 nm thick CPL material deposited on a silicon wafer was tested using an ES01 ellipsometer from BEIJING ELLITOP to obtain the refractive index). The device was transferred back to the glovebox and encapsulated with a glass lid to complete the device.

### Example 3-2: Preparation of an Organic Electroluminescent Device 200 Shown in FIG. 2

This example adopted the same preparation method as Example 3-1, except that Compound H-176 and Compound 72 (96:4, 100 Å) were co-deposited for use as a hole injection layer (HIL), and Compound H-176 (1210 Å) was deposited for use as a hole transporting layer (HTL) and a microcavity length adjustment layer.

### Comparative Example 3-1: Preparation of an Organic Electroluminescent Device 100 Shown in FIG. 1

This comparative example adopted the same preparation method as Example 3-1, except that Compound HT1 and Compound 70 (98:2, 100 Å) were co-deposited for use as a hole injection layer (HIL) 102, Compound HT1 (1160 Å) was deposited for use as a hole transporting layer (HTL) and a microcavity length adjustment layer, and Compound H-176 (50 Å) was deposited for use as an electron blocking layer (EBL) 104.

### Comparative Example 3-2: Preparation of an Organic Electroluminescent Device 200 Shown in FIG. 2

This comparative example adopted the same preparation method as Example 3-1, except that Compound H-176 and Compound HT (98:2, 100 Å) were co-deposited for use as a hole injection layer (HIL).

### Comparative Example 3-3: Preparation of an Organic Electroluminescent Device 100 Shown in FIG. 1

This comparative example adopted the same preparation method as Example 3-2, except that Compound HT1 and Compound 72 (98:2, 100 Å) were co-deposited for use as a hole injection layer (HIL), Compound HT1 (1160 Å) was deposited for use as a hole transporting layer (HTL) and a microcavity length adjustment layer, and Compound H-176 (50 Å) was deposited for use as an electron blocking layer (EBL).

Detailed structures and thicknesses of part of layers of the devices are shown in Table 9. A layer using more than one material is obtained by doping different compounds at their weight ratio as recorded.

TABLE 9
Device structures of some organic layers in Examples
3-1 and 3-2 and Comparative Examples 3-1 to 3-3

| No. | HIL | HTL | EBL | EML |
|---|---|---|---|---|
| Example 3-1 | H-176:70 | H-176 | / | BH:BD |
| | (98:2) (100 Å) | (1210 Å) | | (98:2) (200 Å) |
| Example 3-2 | H-176:72 | H-176 | / | BH:BD |
| | (96:4) (100 Å) | (1210 Å) | | (98:2) (200 Å) |
| Comparative | HT1:70 | HT1 | H-176 | BH:BD |
| Example 3-1 | (98:2) (100 Å) | (1160 Å) | (50 Å) | (98:2) (200 Å) |
| Comparative | H-176:HT | H-176 | / | BH:BD |
| Example 3-2 | (98:2) (100 Å) | (1210 Å) | | (98:2) (200 Å) |
| Comparative | HT1:72 | HT1 | H-176 | BH:BD |
| Example 3-3 | (98:2) (100 Å) | (1160 Å) | (50 Å) | (98:2) (200 Å) |

The structures of the new materials used in the devices are shown as follows:

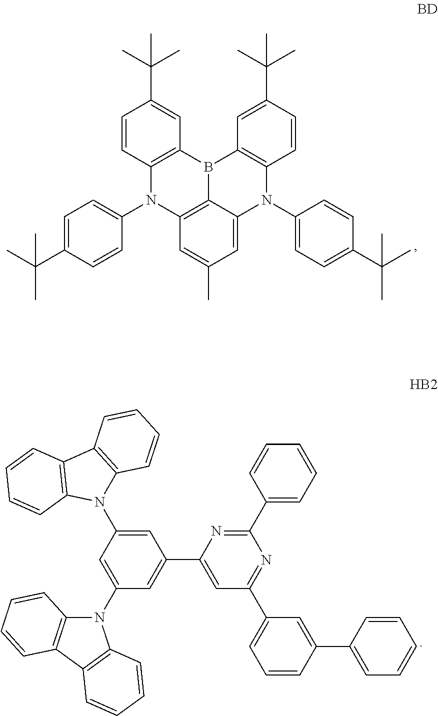 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

TABLE 10
Device performance of Examples 3-1 and 3-2 and Comparative Examples 3-1 to 3-3

| | | | | HOMO | | | | | |
|---|---|---|---|---|---|---|---|---|---|
| | | HOMO | LUMO | Energy | | | | | |
| | Conductivity | Energy | Energy | Level | At 10 mA/cm² | | | | At 80 |
| | of the | Level | Level | of the | | | | Current | mA/cm² |
| | HIL/10⁻⁴ | of the | of the | Host | | | Voltage | Efficiency | LT97 |
| | S/m | HTM/eV | PD/eV | Material/eV | CIEx | CIEy | [V] | (CE)/CIEy | [h] |
| Example 3-1 | 4.6 | −5.27 | −5.17 | −5.53 | 0.140 | 0.041 | 4.1 | 171 | 50 |
| Example 3-2 | 3.6 | −5.27 | −5.17 | −5.53 | 0.140 | 0.041 | 4.1 | 176 | 59 |
| Comparative | 40.4 | −5.09 | −5.17 | −5.53 | 0.139 | 0.042 | 3.9 | 157 | 10 |
| Example 3-1 | | | | | | | | | |
| Comparative | 1.0 | −5.27 | −5.04 | −5.53 | 0.140 | 0.041 | 6.5 | 163 | 2 |
| Example 3-2 | | | | | | | | | |
| Comparative | 32.3 | −5.09 | −5.17 | −5.53 | 0.138 | 0.043 | 3.9 | 160 | 8 |
| Example 3-3 | | | | | | | | | |

It is worth noting that, as is well-known in the industry, for the efficiency of a blue light device in a display panel, the industry generally needs to consider the color factor of the blue light device, that is, adopts CE/CIEy.

The hole injection layer of the device in Example 3-1 is the same as those in Examples 1 and 2 and has good hole injection from the anode to the organic layer. Comparative Example 3-1 is a blue light device structure commonly used in the industry. Compared with Comparative Example 3-1, Example 3-1 has a lifetime increased 5 times and efficiency CE/CIEy improved by 9% from 157 to 171 on the premise of ensuring the same color, and Example 3-1 has better overall performance than Comparative Example 3-1 although the voltage of Example 3-1 is increased by 0.2 V. It is to be noted that the energy level difference between the HOMO energy levels of the HTM (H-176) in Example 3-1 and the host material BH for blue light has an absolute value of 0.26 eV, while the difference is 0.44 eV in Comparative Example 3-1. In Comparative Example 3-1, holes will face a relatively high potential barrier if they directly travel from the HTL to the EML, so the commonly used commercially available device structure is used, where the EBL is added to the device for barrier buffering. The voltage of a device without the EBL is at least 0.5 V higher than the voltage of Comparative Example 3-1, and the device has the greatly reduced efficiency and lifetime. With the greatly improved efficiency and lifetime of the device, Example 3-1 has a voltage comparable to that of Comparative Example 3-1 and increased by only 0.2 V, indicating that the device in Example 3-1 can ensure that holes are effectively transported to the emissive layer.

Similar to those in Comparative Examples 1-2 and 2-2, the hole injection layer in Comparative Example 3-2 uses Compound HT for p-doping and H-176 as the HTM. It can be seen from Table 3 that the conductivity of the hole injection layer is 1×10⁻⁴ S/m, which is lower than that in Example 3-1 so that it can be seen that the hole injection layer has a worse hole injection ability than the HIL in Example 3-1. Similarly, the hole injection ability can be embodied by the energy level difference. Comparative Example 3-2 uses Compound HT with a LUMO energy level of −5.04 eV as the conductive p-type doping material which is doped into Compound H-176 with a HOMO energy level of −5.27 eV for use as the material of the hole injection layer. It can be seen from Table 4 that the energy level difference between the HOMO energy level of the HTM and the LUMO energy level of the PD is 0.23 eV, which is higher than 0.1 eV in Example 3-1. Therefore, the voltage of Comparative Example 3-2 is as high as 6.5 V, its efficiency CE/CIEy is only 163, and its lifetime is only 2 h. Compared with Comparative Example 3-2, Example 3-1 has a voltage reduced by 2.4 V, efficiency CE/CIEy improved by 5% and a lifetime increased 25 times. Here, the energy level difference between the HOMO energy levels of the HTM (H-176) in Comparative Example 3-2 and the host material BH for blue light has an absolute value of 0.26 eV, which is the same as that in Example 3-1, and the difference only lies in that the hole injection layers have different conductivities.

On the basis of Example 3-1, Example 3-2 mainly replaces the PD material in the HIL with Compound 72 and can achieve the same excellent device performance as Example 3-1 in the same blue light device. Similar to the comparison between Example 3-1 and Comparative Example 3-1, Example 3-2 has great advantages in terms of efficiency CE/CIEy and lifetime compared with Comparative Example 3-3. Comparative Example 3-3 also adopts the commonly used commercially available device structure. With the greatly improved efficiency and lifetime of the device, Example 3-2 has a voltage comparable to that of Comparative Example 3-3 and increased by only 0.2 V, indicating that the device in Example 3-2 can ensure that holes are effectively transported to the emissive layer.

As can be seen from the comparison of the above examples and comparative examples, the energy level difference between the HOMO energy levels of the HTM and the host material in the emissive layer and the conductivity of the hole injection layer both have important effects on the device performance, especially the voltage, efficiency and lifetime of the device. Examples 3-1 and 3-2 which satisfy both the conductivity and the energy level difference in the present application can further improve the efficiency and prolong the device lifetime when the CIE are basically unchanged.

To sum up, the organic electroluminescent device with top emission in the present application achieves good device performance, especially a reduced device voltage and a prolonged lifetime, by matching and optimizing the electrical properties of organic function layers, such as the conductivity of the HIL and the energy level difference between the HTM and the host material in the emissive layer.

It should be understood that various embodiments described herein are merely examples and not intended to limit the scope of the present disclosure. Therefore, it is apparent to the persons skilled in the art that the present disclosure as claimed may include variations from specific embodiments and preferred embodiments described herein. Many of materials and structures described herein may be substituted with other materials and structures without departing from the spirit of the present disclosure. It should be understood that various theories as to why the present disclosure works are not intended to be limitative.

What is claimed is:

1. An organic electroluminescent device, comprising:
a substrate;
a first electrode disposed on the substrate;
a second electrode disposed over the first electrode; and
an organic layer disposed between the first electrode and the second electrode;
wherein the first electrode is a material with high reflectivity or a combination of materials with high reflectivity, and the second electrode is a translucent or transparent material or a combination of translucent or transparent materials;
the organic layer comprises a first organic layer, a second organic layer and a third organic layer;
the first organic layer comprises a first organic material and a second organic material;
the second organic layer is made of the second organic material and has a thickness of greater than 80 nm;
the third organic layer is a light-emitting layer comprising at least one light-emitting material and at least one host material;
the first organic layer has a conductivity of greater than 1×10⁻⁴ S/m and less than 1×10⁻² S/m;
an energy level difference between a highest occupied molecular orbital (HOMO) energy level of the second organic material and a HOMO energy level of the at least one host material is less than 0.27 eV; and
one side of the first organic layer is in direct contact with the first electrode, and the other side of the first organic layer is in direct contact with the second organic layer.

2. The organic electroluminescent device according to claim 1, wherein a lowest unoccupied molecular orbital (LUMO) energy level of the first organic material is less than −5.1 eV.

3. The organic electroluminescent device according to claim 1, wherein the HOMO energy level of the second organic material is less than −5.25 eV.

4. The organic electroluminescent device according to claim 1, wherein the second organic layer is in direct contact with the third organic layer.

5. The organic electroluminescent device according to claim 1, wherein the first electrode is selected from the group consisting of Ag, Ti, Cr, Pt, Ni, TiN and combinations thereof with ITO and/or MoOx.

6. The organic electroluminescent device according to claim 1, wherein the second electrode is selected from a Mg—Ag alloy, MoOx, Yb, Ca, ITO, IZO or a combination thereof.

7. The organic electroluminescent device according to claim 1, wherein the energy level difference between the HOMO energy level of the second organic material and the HOMO energy level of the at least one host material is less than 0.25 eV;
preferably, the energy level difference between the HOMO energy level of the second organic material and the HOMO energy level of the at least one host material is less than 0.2 eV.

8. The organic electroluminescent device according to claim 1, wherein an energy level difference between the HOMO energy level of the second organic material and a LUMO energy level of the first organic material is less than 0.23 eV;
preferably, the energy level difference between the HOMO energy level of the second organic material and the LUMO energy level of the first organic material is less than 0.2 eV;
more preferably, the energy level difference between the HOMO energy level of the second organic material and the LUMO energy level of the first organic material is less than or equal to 0.1 eV.

9. The organic electroluminescent device according to claim 1, further comprising an electron injection layer, wherein the electron injection layer is disposed between the third organic layer and the second electrode;
preferably, the electron injection layer comprises the group consisting of Yb, Liq, LiF and combinations thereof.

10. The organic electroluminescent device according to claim 1, wherein the second organic layer has a thickness of greater than 125 nm;
preferably, the second organic layer has a thickness of greater than 150 nm.

11. The organic electroluminescent device according to claim 1, wherein the first organic layer has a conductivity of greater than 2×10⁻⁴ S/m and less than 8×10⁻³ S/m.

12. The organic electroluminescent device according to claim 1, wherein the first organic material has a structure represented by Formula 1: 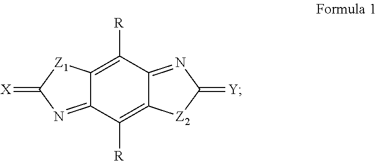
wherein in Formula 1,
X and Y are, at each occurrence identically or differently, selected from NR, CR″R′″, O, S or Se;
Z₁ and Z₂ are, at each occurrence identically or differently, selected from 0, S or Se;
R, R′, R″ and R′″ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
each R may be the same or different, and at least one of R, R, R′ and R′″ is a group having at least one electron withdrawing group; and
in Formula 1, adjacent substituents can be optionally joined to form a ring.

13. The organic electroluminescent device according to claim 1, wherein the second organic material has a structure represented by Formula 2: 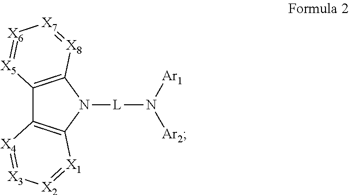
wherein in Formula 2,
X₁ to X₈ are, at each occurrence identically or differently, selected from CR₁ or N;
L is, at each occurrence identically or differently, selected from substituted or unsubstituted arylene having 6 to 30 carbon atoms, substituted or unsubstituted heteroarylene having 3 to 30 carbon atoms or a combination thereof;
Ar₁ and Ar₂ are, at each occurrence identically or differently, selected from substituted or unsubstituted aryl having 6 to 30 carbon atoms or substituted or unsubstituted heteroaryl having 3 to 30 carbon atoms;
R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
in Formula 2, adjacent substituents can be optionally joined to form a ring.

14. A first organic electroluminescent device, comprising:
a substrate;
a first electrode disposed on the substrate;
a second electrode disposed over the first electrode; and
an organic layer disposed between the first electrode and the second electrode;
wherein the first electrode is a material with high reflectivity or a combination of materials with high reflectivity, and the second electrode is a translucent or transparent material or a combination of translucent or transparent materials;
the organic layer comprises a first organic layer, a second organic layer and a third organic layer;
the first organic layer comprises a first organic material and a second organic material;
the second organic layer is made of the second organic material and has a first thickness;
the third organic layer is a light-emitting layer comprising at least one light-emitting material and at least one host material;
the first organic layer has a conductivity of greater than 1×10⁻⁴ S/m and less than 1×10⁻² S/m;
an energy level difference between a highest occupied molecular orbital (HOMO) energy level of the second organic material and a HOMO energy level of the at least one host material is less than 0.27 eV;
a voltage of the first organic electroluminescent device is not higher than 110% of a voltage of a second organic electroluminescent device at the same current density, wherein the second organic electroluminescent device has the same device structure as the first organic electroluminescent device except the following differences:
(1) the first organic layer comprises the first organic material and a third organic material, wherein the third organic material is different from the second organic material;
(2) the second organic layer is made of the third organic material; and
(3) a fourth organic layer is comprised between the second organic layer and the third organic layer, wherein the fourth organic layer is made of the second organic material;
wherein a total thickness of the second organic layer and the fourth organic layer in the second organic electroluminescent device is 90% to 110% of the first thickness in the first organic electroluminescent device.

15. The first organic electroluminescent device according to claim 14, wherein the voltage of the first organic electroluminescent device is not higher than the voltage of the second organic electroluminescent device at the same current density.

16. The first organic electroluminescent device according to claim 14, wherein the HOMO energy level of the second organic material is less than a HOMO energy level of the third organic material in the second organic electroluminescent device.

17. The first organic electroluminescent device according to claim 14, wherein the HOMO energy level of the second organic material is less than −5.25 eV; and/or a lowest unoccupied molecular orbital (LUMO) energy level of the first organic material is less than −5.1 eV.

18. The first organic electroluminescent device according to claim 14, wherein an energy level difference between the HOMO energy level of the second organic material and a LUMO energy level of the first organic material is less than 0.23 eV;
preferably, the energy level difference between the HOMO energy level of the second organic material and the LUMO energy level of the first organic material is less than 0.2 eV;
more preferably, the energy level difference between the HOMO energy level of the second organic material and the LUMO energy level of the first organic material is less than or equal to 0.1 eV.

19. The first organic electroluminescent device according to claim 14, wherein the second organic layer has a thickness of greater than 80 nm;
preferably, the second organic layer has a thickness of greater than 125 nm;
more preferably, the second organic layer has a thickness of greater than 150 nm.

20. A display assembly, comprising the organic electroluminescent device according to any claim 1.
